(19) United States
(12) Patent Application Publication
(10) Pub. No.: US 2026/0092029 A1
(43) Pub. Date: Apr. 2, 2026

(54) LIGAND-ENABLED SCALABLE C-H HYDROXYLATION OF BENZOIC AND PHENYLACETIC ACIDS AT ROOM TEMPERATURE

(71) Applicant: THE SCRIPPS RESEARCH INSTITUTE, La Jolla, CA (US)

(72) Inventors: Jin-Quan YU, San Diego, CA (US); Zhen LI, San Diego, CA (US); Han Seul PARK, San Diego, CA (US)

(21) Appl. No.: 19/109,042

(22) PCT Filed: Sep. 7, 2023
(86) PCT No.: PCT/US2023/073597

§ 371 Date: Mar. 5, 2025

Related U.S. Application Data
(60) Provisional application No. 63/404,246, filed on Sep. 7, 2022.

(51) Int. Cl. C07C 51/367; C07D 235/12; C07D 263/14; C07D 307/83; C07D 307/84; C07D 311/18; C07D 493/04; C07J 1/00

(52) U.S. Cl. CPC C07C 51/367; C07D 235/12; C07D 263/14; C07D 307/83; C07D 307/84; C07D 311/18; C07D 493/04; C07J 1/0059

(57) ABSTRACT

The application discloses industry scalable methods of using bifunctional bidentate pyridone-carboxylic acid ligands, such as 2-methyl-2-(6-oxo-1,6-dihydropyridin-2-yl)propanoic acid, that enable room-temperature Pd-catalyzed C—H hydroxylation of a broad range of benzoic and phenylacetic acids with an industry-compatible oxidant, aqueous hydrogen peroxide, at room temperature. Further disclosed are methods of derivatization of the resulting hydroxylation products, synthesis of polyfluorinated natural products coumestan or pterocarpene from phenol building blocks, and hydroxylation of ibuprofen using this methodology,

### CROSS REFERENCE TO RELATED APPLICATION

This application claims priority to U.S. provisional patent application No. 63/404,246, which was filed on Sep. 7, 2022, and which is hereby incorporated by reference in its entirety.

### GOVERNMENT SUPPORT

This invention was made with government support under GM102265 awarded by the National Institutes of Health. The government has certain rights in the invention.

### FIELD OF THE INVENTION

The application relates to industry scalable methods of using a bifunctional bidentate pyridone-carboxylic acid ligand, such as 2-methyl-2-(6-oxo-1,6-dihydropyridin-2-yl)propanoic acid, that enables room-temperature Pd-catalyzed C—H hydroxylation of a broad range of benzoic and phenylacetic acids with an industry-compatible oxidant, aqueous hydrogen peroxide at room temperature.

### BACKGROUND OF THE INVENTION

With the large number of Pd(II)-catalyzed C—H activation reactions of native substrates developed in the past decade, the development of ligands to enable the use of green oxidants under safe and practical conditions has become an increasingly important challenge.

Phenols are not only common motifs in natural products and bioactive compounds¹⁻³ (Scheme 1a) but also versatile building blocks for organic syntheses⁴. Accordingly, a variety of approaches have been developed to access this important motif, including chemical synthesis, biochemical synthesis⁵ and the controlled decomposition of natural compounds, such as lignin depolymerization⁶. Modern transition metal catalysis has made significant advances in converting a variety of aryl halides or aryl boronic acids into phenols⁷⁻⁹. In principle, the direct hydroxylation of aryl C—H bonds offers an appealing, single-step alternative, but the realization of this strategy has proven challenging. Recently, radical oxygenation of arenes using peroxides were successfully achieved with limited substrate scope^{10,11}. Although the chelation-assisted metal catalysis has emerged as a powerful strategy for selective oxygenation of C—H bond¹²⁻¹⁶, reactions directed by weakly coordinating native functional groups typically require harsh conditions or impractical oxidants.

In order for C—H hydroxylation to be practical and scalable, it is essential that methodologies employ a cheap and environmentally friendly oxidant. Recent studies have demonstrated the feasibility of using molecular oxygen for the direct hydroxylation of C—H bonds¹⁷⁻¹⁹, however, the efficiency, scope, and safety of these methods are unsuitable for industrial scale applications. Since aqueous H₂O₂ has been used in several landmark ton-scale industrial processes including the HPPO (converting propene to propylene oxide) and ε-Caprolactam processes²⁰, we envisioned that Pd-catalyzed C—H hydroxylation with H₂O₂ could provide a practical solution to the synthesis of phenols. However, hydrogen peroxide tends to decompose under the elevated temperature generally required for previously reported Pd(II)-catalyzed C—H activation reactions.

Thus, there is a need in the field for practical and scalable C—H hydroxylation methodologies that employ inexpensive and environmentally friendly oxidants.

### SUMMARY OF THE INVENTION

Herein disclosed is a bifunctional bidentate pyridone-carboxylic acid ligand (Scheme 1) that enables room-temperature Pd-catalyzed C—H hydroxylation of a broad range of benzoic and phenylacetic acids with an industry-compatible oxidant, aqueous hydrogen peroxide (35% H₂O₂). The scalability of this methodology is demonstrated by an 1000 mmol scale reaction of ibuprofen (206 g) using only a 1 mol % Pd(OAc)₂ loading. The utility of this protocol is further illustrated through derivatization of the products and synthesis of polyfluorinated natural product coumestan and pterocarpene from phenol building blocks prepared using this methodology.

The application provides a method of C—H hydroxylation of benzoic and phenylacetic acids, comprising treating a benzoic or phenylacetic acid with a bidentate pyridone-carboxylic acid ligand in aqueous hydrogen peroxide in the presence of Pd(OAc)₂ and a base.

The application provides a method of C—H hydroxylation of phenylacetic acids, wherein the method of C—H hydroxylation of phenylacetic acids occurs according to the following reaction scheme:

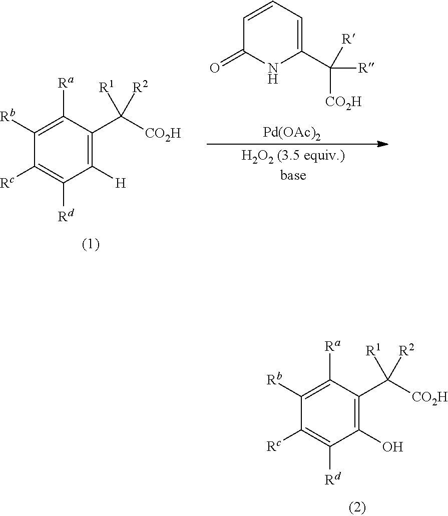

wherein:
R′ and R″ are independently (C₁-C₆)alkyl;
R¹ and R² are independently H, (C₁-C₆)alkyl, cycloalkyl, Ph, benzyl, OH, or —NC(═O)O(C₁-C₆)alkyl;
   or R¹ and R²together form cyclopropyl;
   or R¹ and R^{a} together form a 5- or 6-membered ring saturated or partially unsaturated carbocyclic or heterocyclic ring;
R^{a}, R^{b}, R^{c}, and R^{d} are independently H, halo, —(C₁-C₆)alkyl, halo (C₁-C₆)alkyl, hydroxy (C₁-C₆)alkyl, —O(C₁-C₆)alkyl, —(C₁-C₆)alkyl-O—(C₁-C₆)alkyl, —(C₁-C₆)alkyl-OC(═O)—(C₁-C₆)alkyl, —(C₁-C₆)alkyl-C(═O)O—(C₁-C₆)alkyl, —NH(C₁-C₆)alkyl, optionally substituted Ph, —C(═O)Ph, —C(═O)(C₁-C₆)alkyl, —NC(═O)(C₁-C₆)alkyl, —NC(═O)O(C₁-C₆)alkyl, —CF₃, —CN, —NO₂, cycloalkyl, —(C₁-C₆)alkylcyclocalkyl, or —(C₁-C₆)alkylcyclocalkanone;
   or two adjacent members of R^{a}, R^{b}, R^{c}, and R^{d} together form a 5- or 6-membered aryl or partially unsaturated carbocyclic or heterocyclic ring, optionally substituted with one or more R^{x}; and
   each R^{x} is independently halo, OH, —(C₁-C₆)alkyl, —O(C₁-C₆)alkyl, hydroxy (C₁-C₆)alkyl, —CF₃, —CHF₂, —CH₂F, —CN, —NO₂, cycloalkyl, or —(C₁-C₆)alkylcyclocalkyl.

The application further provides the above method, wherein R′ and R″ are Me.

The application provides a method of C—H hydroxylation of benzoic acids, wherein the method of C—H hydroxylation of benzoic acids occurs according to the following reaction scheme:

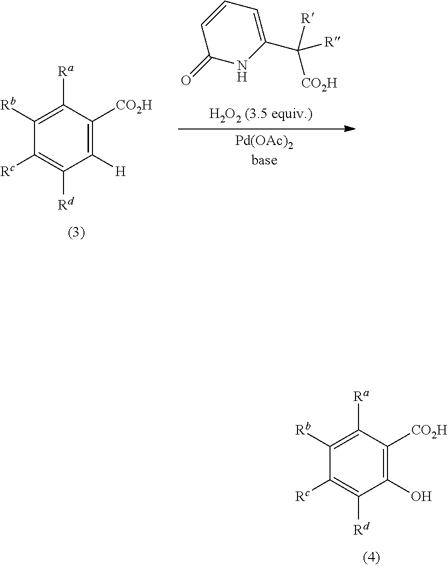

wherein:
R′ and R″ are independently (C₁-C₆)alkyl;
R^{a}, R^{b}, R^{c}, and R^{d} are independently H, halo, —(C₁-C₆)alkyl, halo (C₁-C₆)alkyl, hydroxy (C₁-C₆)alkyl, —O(C₁-C₆)alkyl, —(C₁-C₆)alkyl-O—(C₁-C₆)alkyl, —(C₁-C₆)alkyl-OC(═O)—(C₁-C₆)alkyl, —(C₁-C₆)alkyl-C(═O)O—(C₁-C₆)alkyl, —NH(C₁-C₆)alkyl, optionally substituted Ph, —C(═O)Ph, —C(═O)(C₁-C₆)alkyl, —NC(═O)(C₁-C₆)alkyl, —NC(═O)O(C₁-C₆)alkyl, —CF₃, —CN, —NO₂, cycloalkyl, —(C₁-C₆)alkylcyclocalkyl, or —(C₁-C₆)alkylcyclocalkanone;
   or two adjacent members of R^{a}, R^{b}, R^{c}, and R^{d} together form a 5- or 6-membered aryl or partially unsaturated carbocyclic or heterocyclic ring, optionally substituted with one or more R^{x}; and
   each R^{x} is independently halo, OH, —(C₁-C₆)alkyl, —O(C₁-C₆)alkyl, hydroxy (C₁-C₆)alkyl, —CF₃, —CHF₂, —CH₂F, —CN, —NO₂, cycloalkyl, or —(C₁-C₆)alkylcyclocalkyl.

The application further provides the above method, wherein R′ and R″ are Me.

The application further provides a method of preparing trifluorinated coumestan and pterocarpene according to the following reaction scheme:

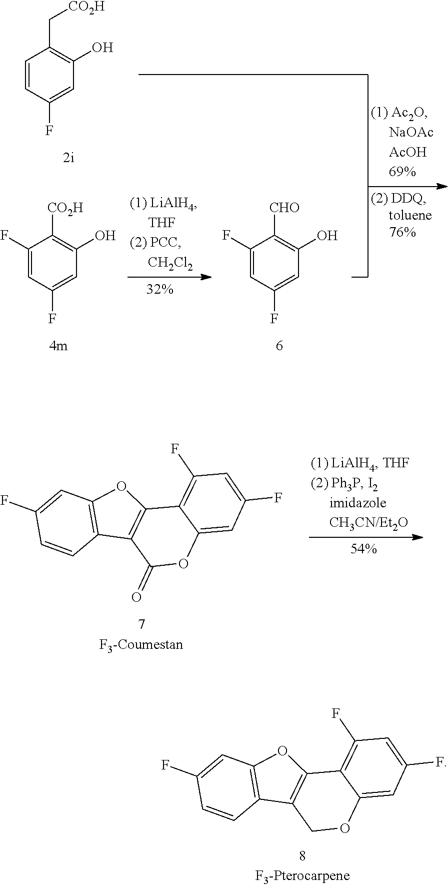 [SMILES: O=C(O)Cc1ccc(F)cc1O ; O=C(O)c1c(O)cc(F)cc1F ; O=Cc1c(O)cc(F)cc1F ; O=c1oc2cc(F)cc(F)c2c2oc3cc(F)ccc3c12 ; Fc1cc(F)c2c(c1)OCc1c-2oc2cc(F)ccc12]

The application further provides a method of hydroxylating ibuprofen according to the following reaction scheme:

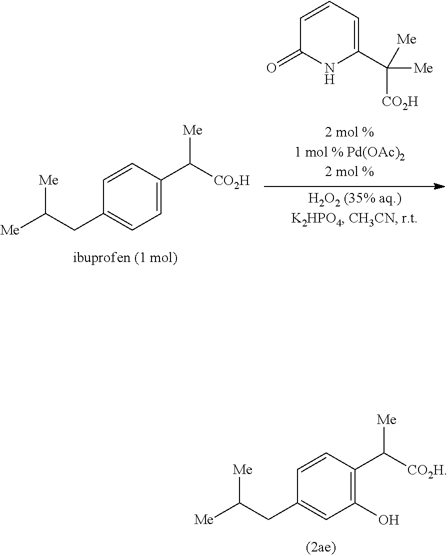 [SMILES: CC(C)(C(=O)O)c1cccc(=O)[nH]1 ; CC(C)Cc1ccc(C(C)C(=O)O)cc1 ; CC(C)Cc1ccc(C(C)C(=O)O)c(O)c1]

### DETAILED DESCRIPTION OF THE INVENTION

The application discloses the development of a bifunctional bidentate pyridone-carboxylic acid ligand, 2-methyl-2-(6-oxo-1,6-dihydropyridin-2-yl)propanoic acid, that enables room-temperature C(sp²)-H hydroxylation of native carboxylic acid substrates with practical, aqueous H₂O₂ as the sole oxidant (Scheme 1c). This new protocol provides an efficient synthetic route to access a wide range of phenols, the synthetic utility of which was demonstrated through the synthesis of two polyfluorinated natural products. Importantly, the reaction can be efficiently scaled up, as highlighted by the synthesis of 179 grams of ortho-hydroxylated ibuprofen. Preliminary mechanistic studies demonstrate that ligand is crucial for accelerating C—H cleavage at room temperature, which prevent the decomposition of hydrogen peroxide.

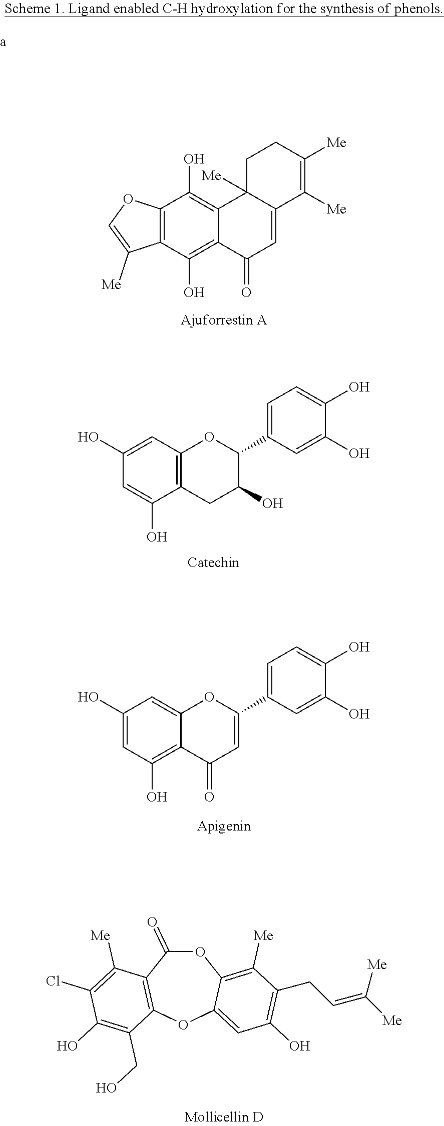

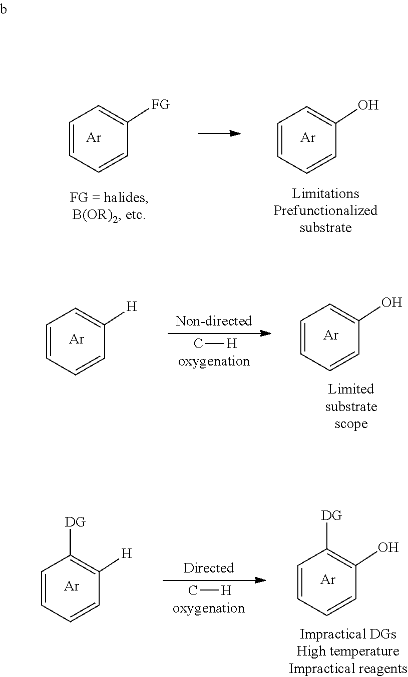

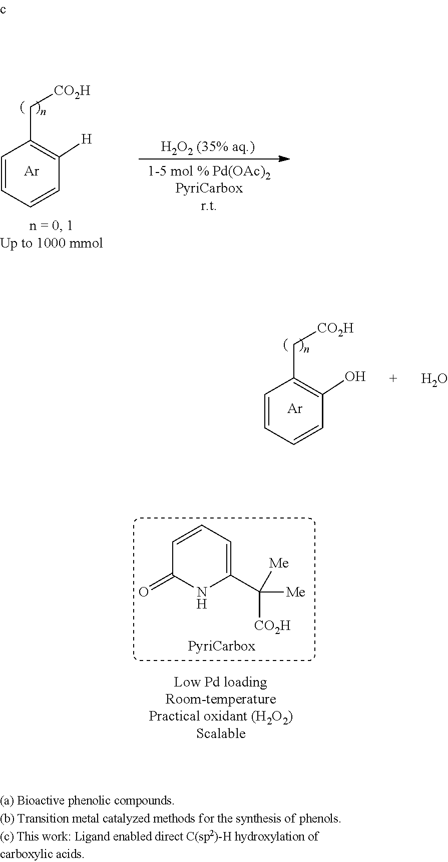

Itoh et al. has reported Pd(II) catalyzed the strongly coordinating pyridine directed C(sp²)-H hydroxylation using hydrogen peroxide²¹. Given our goal of developing a practical C—H hydroxylation reaction, we were interested in developing C—H hydroxylation directed by common, weakly coordinating native directing groups. We were particularly interested in carboxylic acid directing groups due to the abundant sources and versatile conversions. We began our investigation of C(sp²)-H hydroxylation using 4-trifluorometylphenylacetic acid (1a) as a model substrate with 2 mol % Pd(OAc)₂ loading at a 1.0 mmol scale (Table 1).

TABLE 1

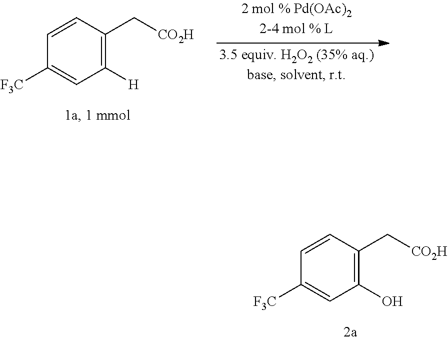 [SMILES: O=C(O)Cc1ccc(C(F)(F)F)cc1 ; O=C(O)Cc1ccc(C(F)(F)F)cc1O]

L1

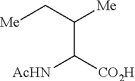 [SMILES: CCC(C)C(NC(C)=O)C(=O)O]

L2

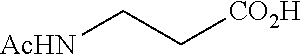

L3

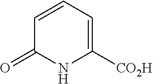

L4

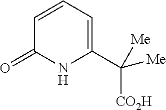 [SMILES: CC(C)(C(=O)O)c1cccc(=O)[nH]1]

L5

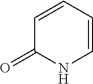

L6

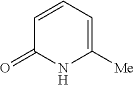

L7

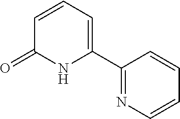 [SMILES: O=c1cccc(-c2ccccn2)[nH]1]

L8

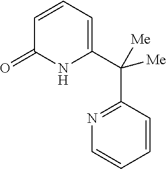 [SMILES: CC(C)(c1ccccn1)c1cccc(=O)[nH]1]

| Entry | Ligand | Base | Solvent | Yield (%) |
|---|---|---|---|---|
| 1^{a} | L1 | K₂HPO₄ | DMA | 37 |
| 2 | L1 | K₂HPO₄ | DMA | 0 |
| 3 | L2 | K₂HPO₄ | DMA | 0 |
| 4 | L3 | K₂HPO₄ | DMA | 0 |
| 5 | L4 | K₂HPO₄ | DMA | 65 |
| 6 | L5 | K₂HPO₄ | DMA | trace |
| 7 | L6 | K₂HPO₄ | DMA | trace |
| 8^{b} | L7 | K₂HPO₄ | DMA | <5 |
| 9^{b} | L8 | K₂HPO₄ | DMA | 0 |
| 10 | No L | K₂HPO₄ | DMA | 0 |
| 11 | L4 | KHCO₃ | DMA | 80 |
| 12 | L4 | K₂HPO₄ | CH₃CN | 86 |

Optimization of the C(sp²)—H hydroxylation using hydrogen peroxide. 4-Trifluoro-phenylacetic acid (1.0 mmol), Pd(OAc)₂ (2 mol %), ligand (4 mol %), H₂O₂ (35% aqueous solution, 3.5 equiv.), base (1.5 equiv.) in solvent (3.0 mL) r.t., 24 h. Yields were determined by ¹H NMR using CH₃NO₂ as the internal standard.
^{a}90° C.
^{b}2 mol % ligand.

Mono N-protected amino acid ligand (MPAA) L1 was chosen for preliminary screening as this ligand is known to accelerate C—H bond cleavage with phenyl acetic acid substrates by direct participation of the acetylamino motif (NHAc) as an internal base during the concerted metalation-deprotonation (CMD) step^{22,23}. To our delight, L1 enabled the formation of the desired ortho-hydroxylated product 2a in 37% yield when using N,N-dimethylacetamide (DMA) as the solvent and H₂O₂(35% aq.) as the oxidant at 90° C. (entry 1). Experiments show that Pd(II) salts decompose 85% of the H₂O₂ at 90° C. within 20 min (see Supplementary Information). We reasoned that the key to improve this reaction is to find a ligand that can ensure Pd(II) catalyst activates C—H preferentially than H₂O₂. Although the decomposition of H₂O₂ was not observed under the reaction conditions at room temperature, MPAA ligands L1 and L2 proved incapable of enabling C—H hydroxylation at room temperature (entries 2-3). Therefore, it was crucial to develop a new ligand that would enable C—H activation at room temperature. Recently, 2-pyridones have emerged as particularly effective ligands that play analogous role to NHAc for challenging Pd(II) catalyzed C—H activation reactions.²⁴⁻²⁶ We hypothesized that incorporation of the 2-pyridone group into the MPAA scaffold in place of NHAc might enable the C—H activation at mild reaction temperatures. Consequently, we designed and tested the pyridone-containing MPAA analogs L3 and L4 (PyriCarbox). While the five membered chelate L3 did not afford the desired product (entry 4); surprisingly, the six-membered chelate L4 afforded 2a in a 65% yield under the room temperature conditions (entry 5). We propose that the increased flexibility of the six-membered chelate may compensate for the rigidity of the planar 2-pyridone motif, allowing the ligand to adopt a favorable conformation for pyridone-assisted C—H cleavage step. When monodentate 2-pyridone ligand L5 and L6 are applied, only trace amount of product was observed, demonstrating the importance of bidentate nature of the ligand (entries 6-7). Interestingly, L7 and L8, two recently developed effective bidentate pyridone-pyridine ligands for C—H activation reactions^{18,25} were not effective (entries 8-9), indicating the importance of retaining a carboxylic acid motif in the bidentate ligand. A control experiment without ligand clearly indicates that ligand plays a key role for this reaction (entry 10). Further optimization with L4 revealed that the base and solvent combinations of KHCO₃ with DMA (entry 11) and K₂HPO₄ with CH₃CN (entry 12) afforded the product in excellent yields.

Having determined optimal conditions, we subjected a wide range of phenylacetic acids to the hydroxylation reaction (Table 2). Phenylacetic acids containing electron-withdrawing (1a, 1e-1k) or electron-donating (1b, 1c)para-substituents, as well as unsubstituted (1d) all provided the corresponding products in high yields. Substrates with various substitution patterns (1l-1q) were also smoothly hydroxylated in high yields, affording the hydroxylated products at the less hindered positions. In addition, the α-substituents of carboxylic acids (1r-1u) were also tolerated, generating the corresponding hydroxylated products. It is noteworthy that hydroxylations of biologically active molecules such as mandelic acids (1v, 1w), protected phenylglycine (1x) and tropic acid (1y) were feasible providing expedient access to phenol derivatives. The phenylacetic acids with tetralin skeleton (1z), dibenzofuran (1aa) and α-quaternary centers (1ab-1ad) were also compatible. Interestingly, the reactions are highly selective for C(sp²)-H bonds, leaving potentially reactive cyclopropyl β-C(sp³)-H bonds intact (2ac, 2ad). Late-stage modification of the existing drug molecules is a powerful approach to rapidly optimize bioactivity of lead compounds. Various anti-inflammatory drugs such as ibuprofen (1ae), ketoprofen (1af), flurbiprofen (1ag), loxoprofen (1ai), and naproxen (1aj) were all successfully hydroxylated with the new practical method. Other pharmaceuticals, including actarit (1ah) and itanapraced (1ak), were also hydroxylated in high yields and with high regioselectivity. Likewise, the complex phenylacetic acid derived from estrone (1al) was hydroxylated at the ortho-position in good yield.

Table 2

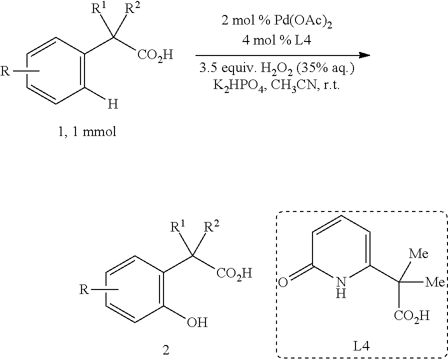

Phenylacetic acids

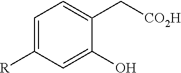

R = CF₃, 80% (2a)
R = Me, 87% (2b)
R = OMe^{a}, 73% (2c)
R = H, 85% (2d)
R = Ph^{a}, 79% (2e)
R = Br, 87% (2f)
R = 1^{a}, 66% (2g)
R = Cl, 81% (2h)
R = F^{a}, 72% (2i)
R = CN^{b}, 74% (2j)
R = NO₂^{b}, 70% (2k)

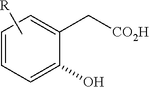

R = 2-Me, 86% (2l)
R = 3-Me, 79% (2m)
R = 2,3-Me₂, 71% (2n)
R = 3-Br, 83% (2o)
R = 3-Cl, 85% (2p)

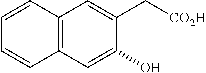

82%^{a} (2q)

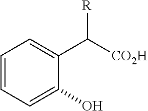

R = Me, 88% (2r)
R = Cy, 74% (2s)
R = Bn, 71% (2t)

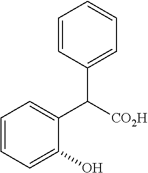

59%^{a} (2u)

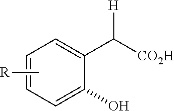

R = H, 83% (2v)
R = 4-Br^{b}, 47% (2w)

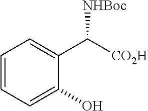

58% (2x)
Mandelic acids

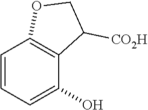

from Tropic acid
75% (2y)

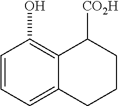

64% (2z)

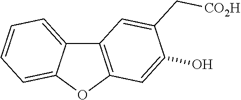

66%^{a} (2aa)

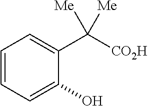

80%^{c} (2ab)

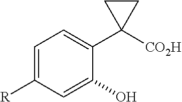

R = H, 78% (2ac)
R = Cl, 56% (2ad)
Anti-inflammatory drugs and bioactive molecules

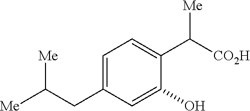

from Ibuprofen
88% (2ae)

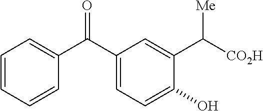

from Ketoprofen
56% (2af)

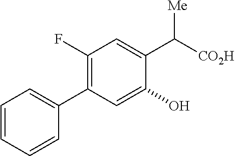

from Flurbiprofen
67% (2ag)

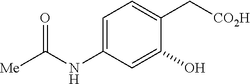

from Actarit
67%^{a} (2ah)

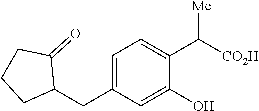 [SMILES: CC(C(=O)O)c1ccc(CC2CCCC2=O)cc1O]

from Loxoprofen
75% (2ai)

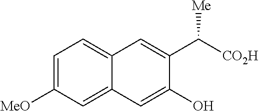 [SMILES: COc1ccc2cc([C@H](C)C(=O)O)c(O)cc2c1]

from Naproxen
65%^{a} (2aj)

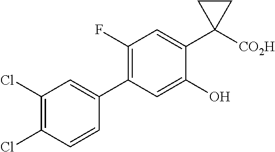 [SMILES: O=C(O)C1(c2cc(F)c(-c3ccc(Cl)c(Cl)c3)cc2O)CC1]

from Itanapraced
74%^{d} (2ak)

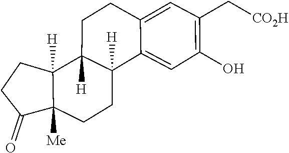

from Estrone
78%^{d} (2al)
Ligand enabled C(sp²)—H hydroxylation of phenylacetic acids. The reaction was performed with carboxylic acid (1 mmol), Pd(OAc)₂ (2 mol %), L4 (4 mol %), H₂O₂ (35% aqueous solution, 3.5 equiv.), and K₂HPO₄ (1.5 equiv.) in CH₃CN (3.0 mL), r.t., 24 h. Isolated yields.
^{a}KHCO₃ (2 mmol) instead of K₂HPO₄, DMA instead of CH₃CN.
^{b}60° C.
^{c}Isolated yield based on the corresponding lactone.
^{d}0.5 mmol scale.

This discovery of ligand-enabled C(sp²)-H hydroxylation of phenylacetic acid substrates led us to test whether this methodology is also applicable to another important class of substrates, benzoic acids (Table 3). It is worth noting that a single catalyst is often not compatible with both phenylacetic and benzoic acid scaffolds. Surprisingly, salicylic acid (4a) was obtained in high yield when benzoic acid was subjected to the similar conditions, differing only in the use of potassium phosphate dibasic trihydrate (K₂HPO₄·3H₂O) as the base. Alkyl or aryl substituted benzoic acids (3b-3f) were effective substrates, affording the corresponding mono-hydroxylated products in good to excellent yields. The reaction was selective for C(sp²)-H hydroxylation with substrate 3d, for which monodentate 2-pyridone ligands have previously been reported to effect benzylic C(sp³)-H activation²⁷. 1-Naphthoic acid (3g) was successfully hydroxylated on 2-position under the reaction conditions without decarboxylation¹⁷. A higher temperature of 60° C. was required for substrate with electron-withdrawing trifluoromethyl group to achieve good yield (3h). Apparently, no significant H₂O₂ decomposition occurred at this relatively mild temperature. Methoxy and cyclic ether substituted benzoic acids (3i-3k) were also compatible substrates. For dihydrobenzofuran carboxylic acid substrate 3j, both regioisomers were obtained in 1:1 ratio (4j and 4j′). NSAID compound diflunisal (4l) could be synthesized from corresponding benzoic acid (3l). However, only trace amount of product was observed for the attempted reaction of 2,4-difluorobenzoic acid (3m). With modified conditions, 2,4-difluorobenzoic acid (3m) was hydroxylated in 72% yield by increasing the Pd(OAc)₂ loading to 5%, and switching solvent to DMA. These new reaction conditions (Conditions B) were then applied to more substrates. We performed high yielding ortho-hydroxylations of benzoic acids with various substituents such as difluoro (3m-3o), fluoro (3p), nitro (3q), acetyl (3r), methoxy (3s) and 4-toluoyl (3u) groups. An ortho-bromo group remained intact during the selective C(sp²)-H hydroxylation of substrate 3t. A hydroxy group was introduced successfully to NSAID drug tolfenamic acid (3v). Estrone derived benzoic acid compound 3w was also hydroxylated in 61% yield.

TABLE 3

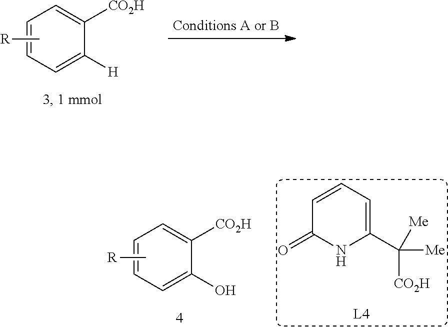

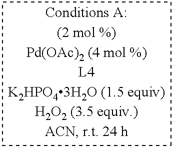

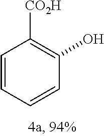

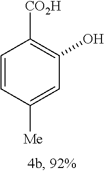

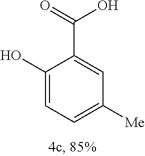 [SMILES: Cc1ccc(O)c(C(=O)O)c1]

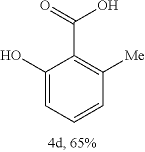 [SMILES: Cc1cccc(O)c1C(=O)O]

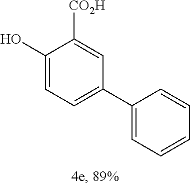 [SMILES: O=C(O)c1cc(-c2ccccc2)ccc1O]

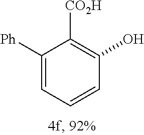

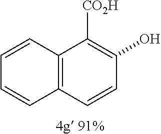

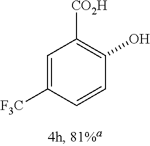

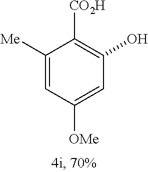

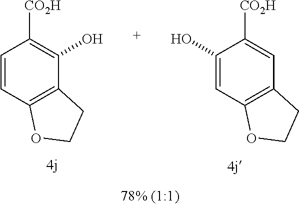

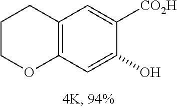

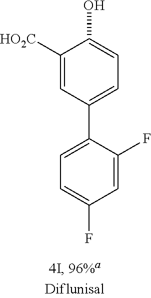

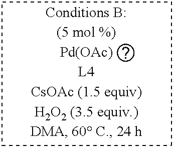

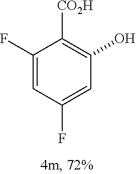

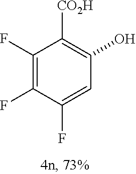

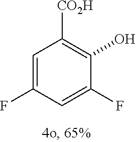

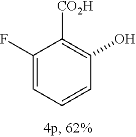

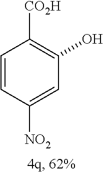

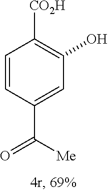

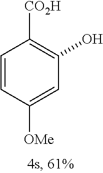

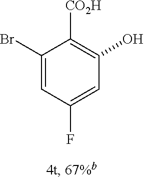

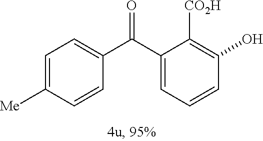

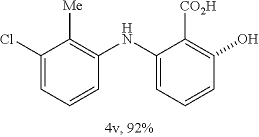

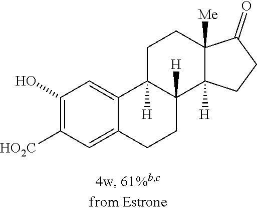

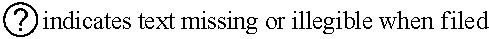

Ligand enabled C(sp₂)—H hydroxylation of benzoic acids. The reaction was performed with carboxylic acid (1 mmol). Conditions A: Pd(OAc)₂ (2 mol %), L4 (4 mol %), H₂O₂ (35% aqueous solution, 3.5 equiv.), and K₂HPO₄•3H2O (1.5 equiv.) in CH₃CN (3.0 mL), r.t. 24 h., Conditions B: Pd(OAc)₂ (5 mol %), L4 (10 mol %), H₂O₂ (35% aqueous solution, 3.5 equiv.), and CsOAc (1.5 equiv.) in DMA (3.0 mL), 60° C. 24 h. Isolated yields.
^{a}60 °C,
^{b}K₂HPO₄•3H₂O instead of CsOAc, 48 h.,
^{c}0.5 mmol scale, r.t.

The robustness and scalability of the hydroxylation reaction was demonstrated through the large-scale reactions of several substrates (Scheme 2a). A mole-scale (206 g) reaction of ibuprofen at room temperature resulted in the formation of 179.2 g of 2ae (81% yield) even with lower loading of 1 mol % Pd(OAc)₂. Similarly, large-scale reactions of 1a and 1d exhibited good yields. The hydroxylation of benzoic acids also proved to be highly scalable, with an 100 mmol scale reaction of benzoic acid (3a) affording 10.6 g of product 4a (77% yield). These results show the potential of this methodology in industrial processes. To showcase the synthetic utility of the reaction, the carboxyl acid directing group of the hydroxylated products were derivatized to various functional groups (Scheme 2b). 2-Hydroxy phenylacetic acids (2a and 2d) could be converted to the corresponding lactones (5a, 5b) and hydrobenzofurans (5c, 5d) through cyclization. Carboxylic acid directing groups were transformed to heterocycles such as the oxazoline (5e) and the benzimidazole (5f). In addition, we sought to demonstrate the utility of the hydroxylation reaction through the synthesis of analogs of natural products. The coumestans and pterocarpans are found in nature, some of which have significant biological activities²⁸. Since the presence of fluorine substituent can uniquely impact the biological and physical properties of compounds, we embarked on the synthesis of unnatural fluorinated coumestan and pterocarpan using building blocks prepared from the title C(sp²)-H hydroxylation reaction (Scheme 2c). The fluorinated precursors 2i and 4m were synthesized in one step from the commercially available carboxylic acids. Salicyl aldehyde 6 was synthesized from 4m in 2 steps. A Perkin reaction of 2i and 6 followed by oxidation afforded trifluorinated coumestan derivative 7 in good yield. We were also able to prepare the trifluorinated version of pterocarpene 8 by reduction of 7 followed by ring closure.

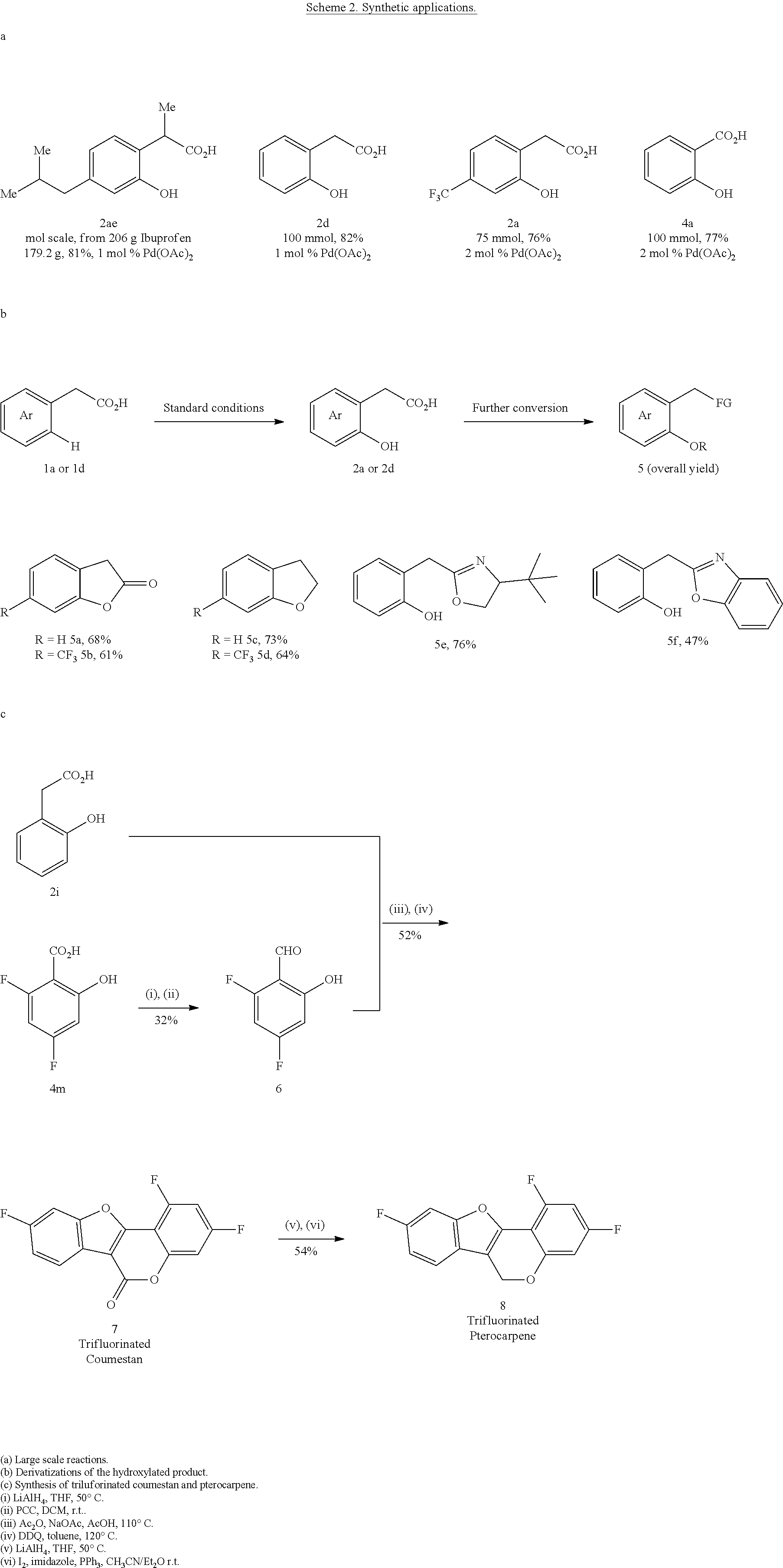

Preliminary mechanistic studies were conducted to gain further insight into the roles of the reaction components. A deuterium labeling experiments of 1d with D₂O in the presence of L4 resulted in 85% deuterium incorporation into the ortho-positions. However, in the absence of L4 no H/D exchange was observed (Scheme 3a). These results suggest that the bidentate pyridone-carboxylic acid ligand is critical for enabling room temperature C—H activation. The kinetic isotope effect (KIE) was measured though parallel experiments of 2d and 2d-d₅ (see Supplementary Information for details). The measured k_{H}/k_{D} value of 1.08 suggests that C—H cleavage is fast and not the rate-limiting step (Scheme 3b). The palladacycle intermediate (9) prepared from 2-methyl benzoic acid was subjected to hydroxylated conditions and provided the product in 39% yield, consistent with the proposal that palladacycles such as 9 are active intermediates in the reaction (Scheme 3c). Based on these observations and previous reports²⁹⁻³¹, a Pd(II)/Pd(IV) catalytic cycle is proposed (Scheme 3d). Following ligand enabled C—H cleavage, oxidative addition of H₂O₂ to Pd(II) forms the high valent Pd(IV) species which subsequently undergoes reductive elimination to form the hydroxylated product. Ligand exchange with 2HX forms water as the sole byproduct and regenerates the catalyst PdX₂.

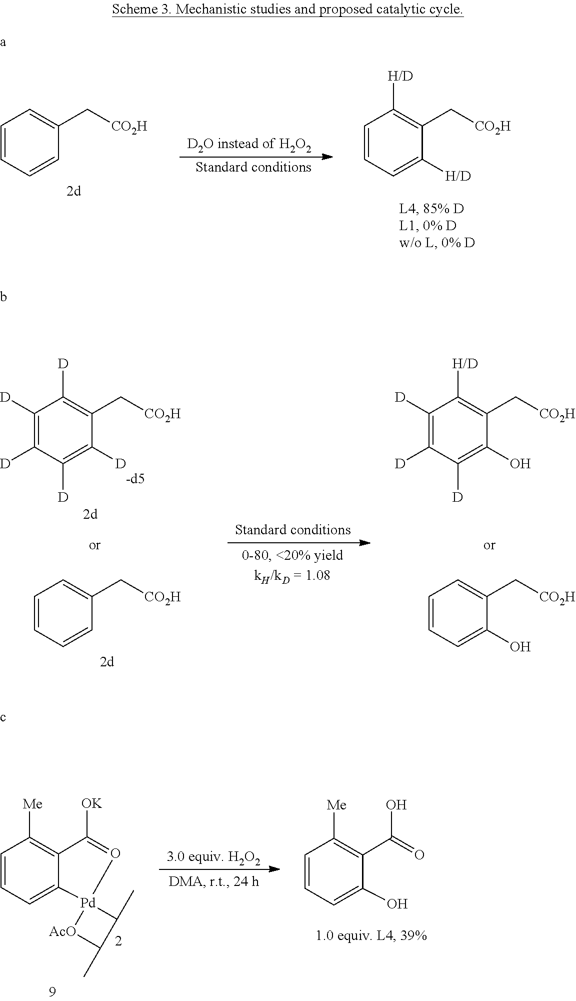

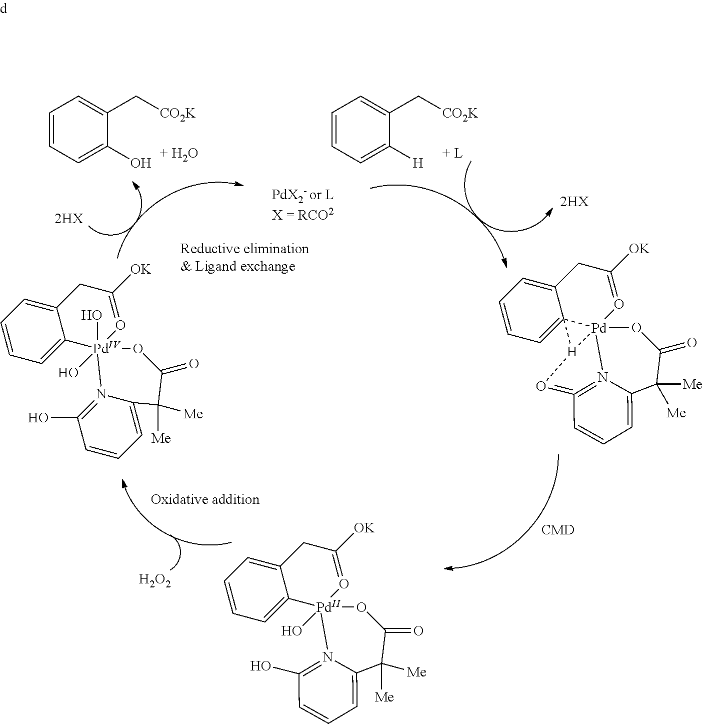

In summary, a practical C(sp²)-H hydroxylation of carboxylic acid substrates using hydrogen peroxide aqueous solution as the hydroxylating reagent, enabled by a newly developed bifunctional pyridone-carboxylic acid (PyriCarbox) ligand has been developed. Mechanistic studies indicate that this ligand scaffold play a crucial role in achieving room temperature C—H activation, thus permitting the development of mild reaction conditions which preclude the decomposition of hydrogen peroxide. With this new protocol, a wide range of phenylacetic acids and benzoic acids were successfully hydroxylated, providing the corresponding phenols. The practicality and scalability of the reaction was highlighted by large scale examples including an 1000 mmol scale reaction of ibuprofen, suggesting the transformation may have the potential to be applied to industrial scale processes. Furthermore, the derivatizations of phenol products and synthesis of trifluorinated coumestan and pterocarpene were achieved.

### REFERENCES

1. Albuquerque, B. R., Heleno, S. A., Oliveira, M. B. P. P., Barros, L. & Ferreira, I. C. F. R. Phenolic compounds: current industrial applications, limitations and future challenges. Food Funct. 12, (2021).
2. Quideau, S., Deffieux, D., Douat-Casassus, C. & Pouysegu, L. Plant polyphenols: chemical properties, biological activities, and synthesis. Angew. Chem. Int. Ed. 50, 586-621 (2011).
3. Scott, K. A., Cox, P. B. & Njardarson, J. T. Phenols in pharmaceuticals: Analysis of a recurring motif. J. Med. Chem. 65, 7044-7072 (2022).
4. Qiu, Z. H. & Li, C. J. Transformations of less-activated phenols and phenol derivatives via C—O cleavage. Chem. Rev. 120, 10454-10515 (2020).
5. Ullrich, R. & Hofrichter, M. Enzymatic hydroxylation of aromatic compounds. Cell. Mol. Life. Sci. 64, 271-293 (2007).
6. Sun, Z. H., Fridrich, B., de Santi, A., Elangovan, S. & Barta, K. Bright side of lignin depolymerization: toward new platform chemicals. Chem. Rev. 118, 614-678 (2018).
7. Maleczka, R. E. Jr., Shi, F., Holmes, D. & Smith, M. R. III. C—H activation/borylation/oxidation: a one-pot unified route to meta-substituted phenois bearing ortho-/para-directing groups. J. Am. Chem. Soc. 125, 7792-7793 (2003).
8. Anderson, K. W., Ikawa, T., Tundel, R. E. & Buchwald, S. L. The selective reaction of aryl halides with KOH: Synthesis of phenols, aromatic ethers, and benzofurans. J. Am. Chem. Soc. 128, 10694-10695 (2006).
9. Xia, S. H., Gan, L., Wang, K., Li, Z. & Ma, D. W. Copper-catalyzed hydroxylation of (hetero)aryl halides under mild conditions. J. Am. Chem. Soc. 138, 13493-13496 (2016).
10. Yuan, C. X., Liang Y., Hernandez, T., Berriochoa, A., Houk, K. N. & Siegel, D Metal-free oxidation of aromatic carbon-hydrogen bonds through a reverse-rebound mechanism. Nature 499, 192-196 (2013).
11. Cheng, L. Wang, H., Cai, H., Zhang, J., Gong, X. & Han, W. Iron-catalyzed arene C—H hydroxylation. Science 374, 77-81 (2021).
12. Huang, C. H., Ghavtadze, N., Chattopadhyay, B. & Gevorgyan, V. Synthesis of catechols from phenols via Pd-catalyzed silanol-directed C—H oxygenation. J. Am. Chem. Soc. 133, 17630-17633 (2011).
13. Shan, G., Yang, X., Ma, L. & Rao, Y. Pd-catalyzed C—H oxygenation with TFA/TFAA: expedient access to oxygen-containing heterocycles and late-stage drug modification. Angew. Chem. Int. Ed. 51, 13070-13074 (2012).
14. Mo, F., Trzepkowski, L. J. & Dong, G. Synthesis of ortho-acylphenols through the palladium-catalyzed ketone-directed hydroxylation of arenes. Angew. Chem. Int. Ed. 51, 13075-13079 (2012).
15. Liang, Y.-F., Wang, X., Yuan, Y., Liang, Y., Li, X. & Jiao, N. Ligand-promoted Pd-catalyzed oxime ether directed C—H hydroxylation of arenes. ACS Catal. 5, 6148-6152 (2015).
16. Dong, J., Liu, P. & Sun, P. Palladium-catalyzed aryl C(sp²)-H bond hydroxylation of 2-arylpyridine using TBHP as oxidant. J. Org. Chem. 80, 2925-2929 (2015).
17. Zhang, Y.-H. & Yu, J.-Q. Pd(II)-catalyzed hydroxylation of arenes with 1 atm of O₂ or air. J. Am. Chem. Soc. 131, 14654-14655 (2009).
18. Li, Z., Wang, Z., Chekshin, N., Qian, S., Qiao, J. X., Cheng, P. T., Yeung, K.-S., Ewing, W. R. & Yu, J.-Q. A tautomeric ligand enables directed C—H hydroxylation with molecular oxygen. Science 372, 1452-1457 (2021).
19. Yan, Y., Feng, P., Zheng, Q.-Z., Liang, Y.-F., Lu, J.-F., Cui, Y. & Jiao, N. PdCl₂ and N-hydroxyphthalimide co-catalyzed C(sp²)-H hydroxylation by dioxygen activation. Angew. Chem. Int. Ed. 52, 5827-5831 (2013).
20. Clerici, M. G. & Kholdeeva, O. A. Liquid phase oxidation via heterogeneous catalysis: organic synthesis and industrial applications. (Wiley, 2013).
21. Yamaguchi, T., Yamaguchi, E., Tada, N. & Itoh, A. Direct ortho-hydroxylation of 2-phenylpyridines using palladium(II) chloride and hydrogen peroxide. Adv. Synth. Catal. 357, 2017-2021 (2015).
22. Engle, K. M., Wang, D.-H. & Yu, J.-Q. Ligand-accelerated C—H activation reactions: evidence for a switch of mechanism. J. Am. Chem. Soc. 132, 14137-14151 (2010).
23. Engle, K. M., Thuy-Boun, P. S., Dang, M. & Yu, J.-Q. Ligand-accelerated cross-coupling of C(sp²)-H bonds with arylboron reagents. J. Am. Chem. Soc. 133, 18183-18193 (2011).
24. Wang, P., Verma, P., Xia, G., Qiao, J. X., Tao, S., Cheng, P. T. W., Poss, M. A., Farmer, M. E., Yeung, K.-S. & Yu J.-Q. Ligand-accelerated non-directed C—H functionalization of arenes. Nature 551, 489-404 (2017).
25. Wang, Z., Hu, L., Chekshin, N., Zhuang, Z., Qian, S., Qiao, J. X. & Yu, J.-Q. Ligand-controlled divergent dehydrogenative reactions of carboxylic acids via C—H activation. Science 374, 1281-1285 (2021).
26. Chan, H. S. S., Yang, J.-M. & Yu, J.-Q. Catalyst-controlled site-selective methylene C—H lactonization of dicarboxylic acids. Science 376, 1481-1487 (2022).
27. Qian, S. Q., Li, Z.-Q., Li, M., Wisniewski, S. R., Qiao, J. X., Richter, J. M., Ewing, W. R., Eastgate, M. D., Chen, J. S. & Yu, J.-Q. Ligand-enabled Pd(II)-catalyzed C(sp³)-H lactonization using molecular oxygen as xxidant. Or.gLett. 22, 3960-3963 (2020).
28. Selvam, C., Jordan, B. C., Prakash, S., Mutisya, D. & Thilagavathi, R. Pterocarpan scaffold: a natural lead molecule with diverse pharmacological properties. Eur. J. Med. Chem. 128, 219-236 (2017).
29. Oloo, W., Zavalij, P. Y., Zhang, J., Khaskin, E. & Vedernikov, A. N. Preparation and C—X reductive elimination reactivity of monoaryl Pd^{1V}—X complexes in water (X═OH, OH₂, Cl, Br). J. Am. Chem. Soc. 132, 14400-14402 (2010).
30. Zhuang, Z., Herron, A. N., Fan, Z. & Yu, J.-Q. Ligand-enabled monoselective β-C(sp³)-H acyloxylation of free carboxylic acids using a practical oxidant. J. Am. Chem. Soc. 142, 6769-6776 (2020).
31. Giri, R., Liang, J., Lei, J.-G., Li, J.-J., Wang, D.-H., Chen, X., Naggar, I. C., Guo, C., Foxman, B. M. & Yu, J.-Q. Pd-catalyzed stereoselective oxidation of methyl groups by inexpensive oxidants under mild conditions: A dual role for carboxylic anhydrides in catalytic C—H bond oxidation. Angew. Chem. Int. Ed. 44, 7420-7424 (2005).

### EMBODIMENTS

Embodiment 1. A method of C—H hydroxylation of benzoic and phenylacetic acids, comprising treating a benzoic or phenylacetic acid with a bidentate pyridone-carboxylic acid ligand in aqueous hydrogen peroxide in the presence of Pd(OAc)₂ and a base.

Embodiment 2. The method of Embodiment 1, wherein the method of C—H hydroxylation of phenylacetic acids of Formula (1) occurs according to the following reaction scheme:

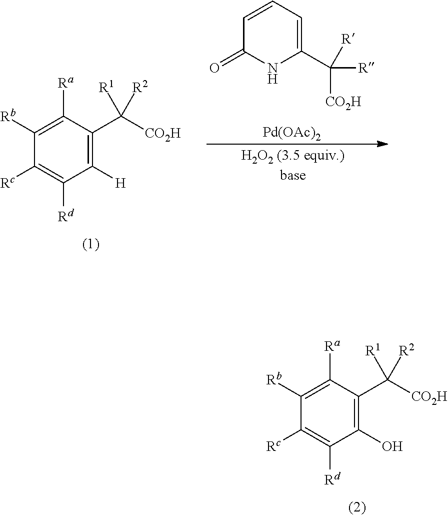

wherein:
R′ and R″ are independently (C₁-C₆)alkyl;
R¹ and R² are independently H, (C₁-C₆)alkyl, cycloalkyl, Ph, benzyl, OH, or —NC(═O)O(C₁-C₆)alkyl;
   or R¹ and R²together form cyclopropyl;
   or R¹ and R^{a} together form a 5- or 6-membered ring saturated or partially unsaturated carbocyclic or heterocyclic ring;
R^{a}, R^{b}, R^{c}, and R^{d} are independently H, halo, —(C₁-C₆)alkyl, halo (C₁-C₆)alkyl, hydroxy (C₁-C₆)alkyl, —O(C₁-C₆)alkyl, —(C₁-C₆)alkyl-O—(C₁-C₆)alkyl, —(C₁-C₆)alkyl-OC(═O)—(C₁-C₆)alkyl, —(C₁-C₆)alkyl-C(═O)O—(C₁-C₆)alkyl, —NH(C₁-C₆)alkyl, optionally substituted Ph, —C(═O)Ph, —C(═O)(C₁-C₆)alkyl, —NC(═O)(C₁-C₆)alkyl, —NC(═O)O(C₁-C₆)alkyl, —CF₃, —CN, —NO₂, cycloalkyl, —(C₁-C₆)alkylcyclocalkyl, or —(C₁-C₆)alkylcyclocalkanone;
   or two adjacent members of R^{a}, R^{b}, R^{c}, and R^{d} together form a 5- or 6-membered aryl or partially unsaturated carbocyclic or heterocyclic ring, optionally substituted with one or more R^{x}; and
   each R^{x} is independently halo, OH, —(C₁-C₆)alkyl, —O(C₁-C₆)alkyl, hydroxy (C₁-C₆)alkyl, —CF₃, —CHF₂, —CH₂F, —CN, —NO₂, cycloalkyl, or —(C₁-C₆)alkylcyclocalkyl.

Embodiment 3. The method of Embodiment 2, wherein R′ is Me and R″ is Me.

Embodiment 4. The method of either Embodiment 2 or Embodiment 3, wherein R¹ is Me.

Embodiment 5. The method of any one of Embodiments 2-4, wherein R² is Me.

Embodiment 6. The method of either Embodiment 2 or Embodiment 3, wherein R¹ is H.

Embodiment 7. The method of Embodiment 6, wherein R² is H.

Embodiment 8. The method of Embodiment 6, wherein R² is OH.

Embodiment 9. The method of Embodiment 6, wherein R² is (C₃-C₆)cycloalkyl.

Embodiment 10. The method of Embodiment 9, wherein R² is cyclohexyl.

Embodiment 11. The method of Embodiment 6, wherein R² is benzyl.

Embodiment 12. The method of Embodiment 6, wherein R² is NBoc.

Embodiment 13. The method of Embodiment 2, wherein R¹ and R²together form cyclopropyl.

Embodiment 14. The method of Embodiment 1, wherein the method of C—H hydroxylation of benzoic acids occurs according to the following reaction scheme:

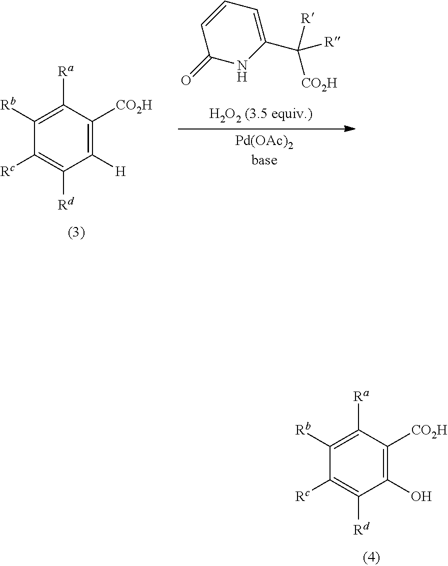

wherein:
R′ and R″ are independently (C₁-C₆)alkyl;
R^{a}, R^{b}, R^{c}, and R^{d} are independently H, halo, —(C₁-C₆)alkyl, halo (C₁-C₆)alkyl, hydroxy (C₁-C₆)alkyl, —O(C₁-C₆)alkyl, —(C₁-C₆)alkyl-O—(C₁-C₆)alkyl, —(C₁-C₆)alkyl-OC(═O)—(C₁-C₆)alkyl, —(C₁-C₆)alkyl-C(═O)O—(C₁-C₆)alkyl, —NH(C₁-C₆)alkyl, optionally substituted Ph, —C(═O)Ph, —C(═O)(C₁-C₆)alkyl, —NC(═O)(C₁-C₆)alkyl, —NC(═O)O(C₁-C₆)alkyl, —CF₃, —CN, —NO₂, cycloalkyl, —(C₁-C₆)alkylcyclocalkyl, or —(C₁-C₆)alkylcyclocalkanone;
   or two adjacent members of R^{a}, R^{b}, R^{c}, and R^{d} together form a 5- or 6-membered aryl or partially unsaturated carbocyclic or heterocyclic ring, optionally substituted with one or more R^{x}; and
   each R^{x} is independently halo, OH, —(C₁-C₆)alkyl, —O(C₁-C₆)alkyl, hydroxy (C₁-C₆)alkyl, —CF₃, —CHF₂, —CH₂F, —CN, —NO₂, cycloalkyl, or —(C₁-C₆)alkylcyclocalkyl.

Embodiment 15. The method of Embodiment 14, wherein R′ is Me and R″ is Me.

Embodiment 16. The method of any one of Embodiments 2-15, wherein R^{c} is (C₁-C₆)alkyl.

Embodiment 17. The method of Embodiment 16, wherein R^{c} is Me.

Embodiment 18. The method of any one of Embodiments 2-15, wherein R^{c} is halo.

Embodiment 19. The method of Embodiment 18, wherein R^{c} is F.

Embodiment 20. The method of Embodiment 18, wherein R^{c} is Cl.

Embodiment 21. The method of Embodiment 18, wherein R^{c} is Br.

Embodiment 22. The method of any one of Embodiments 2-15, wherein R^{c} is —O(C₁-C₆)alkyl.

Embodiment 23. The method of Embodiment 22, wherein R^{c} is —OMe.

Embodiment 24. The method of any one of Embodiments 2-15, wherein R^{c} is —CF₃.

Embodiment 25. The method of any one of Embodiments 2-15, wherein R^{c} is optionally substituted Ph.

Embodiment 26. The method of any one of Embodiments 2-25, wherein R^{b} is (C₁-C₆)alkyl.

Embodiment 27. The method of Embodiment 26, wherein R^{b} is Me.

Embodiment 28. The method of any one of Embodiments 2-25, wherein R^{b} is halo.

Embodiment 29. The method of Embodiment 28, wherein R^{b} is F.

Embodiment 30. The method of Embodiment 28, wherein R^{b} is Cl.

Embodiment 31. The method of Embodiment 28, wherein R^{b} is Br.

Embodiment 32. The method of Embodiment 28, wherein R^{b} is I.

Embodiment 33. The method of any one of Embodiments 2-25, wherein R^{b} is —CF₃.

Embodiment 34. The method of any one of Embodiments 2-25, wherein R^{b} is —O(C₁-C₆)alkyl.

Embodiment 35. The method of Embodiment 34, wherein R^{b} is —OMe.

Embodiment 36. The method of any one of Embodiments 2-25, wherein R^{b} is optionally substituted Ph.

Embodiment 37. The method of any one of Embodiments 2-36, wherein R^{a}is (C₁-C₆)alkyl.

Embodiment 38. The method of Embodiment 37, wherein R^{a}is Me.

Embodiment 39. The method of any one of Embodiments 2-36, wherein R^{a}is halo.

Embodiment 40. The method of Embodiment 39, wherein R^{a}is F.

Embodiment 41. The method of Embodiment 39, wherein R^{a}is Cl.

Embodiment 42. The method of Embodiment 39, wherein R^{a}is Br.

Embodiment 43. The method of Embodiment 39, wherein R^{a}is I.

Embodiment 44. The method of any one of Embodiments 2-36, wherein R^{a}is —CF₃.

Embodiment 45. The method of any one of Embodiments 2-36, wherein R^{a}is —O(C₁-C₆)alkyl.

Embodiment 46. The method of Embodiment 45, wherein R^{a}is —OMe.

Embodiment 47. The method of any one of Embodiments 2-36, wherein R^{a}is optionally substituted Ph.

Embodiment 48. The method of any one of Embodiments 2-15, wherein R^{a} and R^{b}together form a 5- or 6-membered aryl or partially unsaturated carbocyclic or heterocyclic ring, optionally substituted with one or more R^{x}.

Embodiment 49. The method of Embodiment 48, wherein R^{a} and R^{b} together form Ph optionally substituted with one or more R^{x}.

Embodiment 50. The method of any one of Embodiments 2-15, wherein R^{b} and R^{c} together form a 5- or 6-membered aryl or partially unsaturated carbocyclic or heterocyclic ring an optionally substituted with one or more R^{x}.

Embodiment 51. The method of Embodiment 50, wherein R^{b} and R^{c} together form Ph optionally substituted with one or more R^{x}.

Embodiment 52. The method of Embodiment 2, wherein R¹ and R^{a} together form a 5- or 6-membered ring saturated or partially unsaturated carbocyclic or heterocyclic ring optionally substituted with one or more R^{x}.

Embodiment 53. The method of Embodiment 52, wherein R^{b} and R^{a} together form cyclopentyl optionally substituted with one or more R^{x}.

Embodiment 54. The method of Embodiment 52, wherein R^{b} and R^{a} together form a 5-membered heterocyclic ring optionally substituted with one or more R^{x}.

Embodiment 55. The method any one of Embodiments 2-54, wherein the base is K₂HPO₄.

Embodiment 56. The method of Embodiment 55, wherein the solvent is CH₃CN.

Embodiment 57. The method of Embodiment 55, wherein the solvent is DMA.

Embodiment 58. The method of Embodiment 55, wherein the solvent is DMF.

Embodiment 59. The method of Embodiment 55, wherein the solvent is acetone.

Embodiment 60. The method of Embodiment 55, wherein the solvent is NMP, t-Amyl-OH, or DCE.

Embodiment 61. The method any one of Embodiments 2-54, wherein the base is CsOAc.

Embodiment 62. The method of Embodiment 61, wherein the solvent is DMA.

Embodiment 63. The method of Embodiment 61, wherein the solvent is CH₃CN.

Embodiment 64. The method any one of Embodiments 2-54, wherein the base is KOAc.

Embodiment 65. The method any one of Embodiments 2-54, wherein the base is NaOAc.

Embodiment 66. The method either Embodiment 64 or 65, wherein the solvent is CH₃CN.

Embodiment 67. The method of Embodiment 2, wherein base is KHCO₃ and the solvent is DMA.

Embodiment 68. The method of Embodiment 2, wherein 1 mmol phenylacetic acid of Formula (1) is treated with 4 mol % 2-methyl-2-(6-oxo-1,6-dihydropyridin-2-yl)propanoic acid, 2 mol % Pd(OAc)₂, 3.5 equiv. H₂O₂, and 1.5 equiv. K₂HPO₄ in CH₃CN at r.t. for approximately 24 h to form the hydroxylated phenyl acetic acid product of Formula (2).

Embodiment 69. The method of embodiment 68, wherein the hydroxylated phenyl acetic acid product of Formula (2) is selected from the group consisting of:
2-(2-hydroxy-4-(trifluoromethyl)phenyl)acetic acid (2a);
2-(2-hydroxy-4-methylphenyl)acetic acid (2b);
2-(2-hydroxyphenyl)acetic acid (2d);
2-(4-bromo-2-hydroxyphenyl)acetic acid (2f);
2-(4-chloro-2-hydroxyphenyl)acetic acid (2h);
2-(2-hydroxy-6-methylphenyl)acetic acid (2l);
2-(2-hydroxy-5-methylphenyl)acetic acid (2m);
2-(6-hydroxy-2,3-dimethylphenyl)acetic acid (2n);
2-(5-bromo-2-hydroxyphenyl)acetic acid (2o);
2-(5-chloro-2-hydroxyphenyl)acetic acid (2p);
2-(2-hydroxyphenyl)propanoic acid (2r);
2-cyclohexyl-2-(2-hydroxyphenyl)acetic acid (2s);
2-(2-hydroxyphenyl)-3-phenylpropanoic acid (2t);
2-hydroxy-2-(2-hydroxyphenyl)acetic acid (2v);
(S)-2-((tert-butoxycarbonyl)amino)-2-(2-hydroxyphenyl)acetic (2x);
4-hydroxy-2,3-dihydrobenzofuran-3-carboxylic acid (2y);
8-hydroxy-1,2,3,4-tetrahydronaphthalene-1-carboxylic acid (2z);
3,3-dimethylbenzofuran-2(3H)-one (2ab′);
1-(2-hydroxyphenyl)cyclopropane-1-carboxylic acid (2ac);
1-(4-chloro-2-hydroxyphenyl)cyclopropane-1-carboxylic acid (2ad);
2-(2-hydroxy-4-isobutylphenyl)propanoic acid (2ae);
2-(5-benzoyl-2-hydroxyphenyl)propanoic acid (2af);
2-(2-fluoro-5-hydroxy-[1,1′-biphenyl]-4-yl)propanoic acid (2ag);
2-(2-hydroxy-4-((2-oxocyclopentyl)methyl)phenyl)propanoic acid (2ai); and
(S)-2-(3-hydroxy-6-methoxynaphthalen-2-yl)propanoic acid (2aj).

Embodiment 70. The method of Embodiment 2, wherein 0.5 mmol phenylacetic acid of Formula (1) is treated with 4 mol % 2-methyl-2-(6-oxo-1,6-dihydropyridin-2-yl)propanoic acid, 2 mol % Pd(OAc)₂, 3.5 equiv. H₂O₂, and 1.5 equiv. K₂HPO₄ in CH₃CN at r.t. for approximately 24 h to form the hydroxylated phenyl acetic acid product of Formula (2).

Embodiment 71. The method of embodiment 70, wherein the hydroxylated phenyl acetic acid product of Formula (2) is selected from the group consisting of: 1-(3′,4′-dichloro-2-fluoro-5-hydroxy-[1,1′-biphenyl]-4-yl)cyclopropane-1-carboxylic acid (2ak); and 2-((8R,9S,13S,14S)-2-hydroxy-13-methyl-17-oxo-7,8,9,11,12,13,14,15,16,17-decahydro-6H-cyclopenta[a]phenanthren-3-yl)acetic acid (2a1).

Embodiment 72. The method of Embodiment 2, wherein 1 mmol phenylacetic acid of Formula (1) is treated with 4 mol % 2-methyl-2-(6-oxo-1,6-dihydropyridin-2-yl)propanoic acid, 2 mol % Pd(OAc)₂, 3.5 equiv. H₂O₂, and 1.5 equiv. K₂HPO₄ in CH₃CN at 60° C. for approximately 24 h to form the hydroxylated phenyl acetic acid product of Formula (2).

Embodiment 73. The method of embodiment 72, wherein the hydroxylated phenyl acetic acid product of Formula (2) is selected from the group consisting of:
2-(4-cyano-2-hydroxyphenyl)acetic acid (2j);
2-(2-hydroxy-4-nitrophenyl)acetic acid (2k); and
2-(4-bromo-2-hydroxyphenyl)-2-hydroxyacetic (2w).

Embodiment 74. The method of Embodiment 2, wherein 1 mmol phenylacetic acid of Formula (1) is treated with 4 mol % 2-methyl-2-(6-oxo-1,6-dihydropyridin-2-yl)propanoic acid, 2 mol % Pd(OAc)₂, 3.5 equiv. H₂O₂, and 2.0 equiv. KHCO₃ in DMA at r.t. for approximately 24 h to form the hydroxylated phenyl acetic acid product of Formula (2).

Embodiment 75. The method of embodiment 74, wherein the hydroxylated phenyl acetic acid product of Formula (2) is selected from the group consisting of:
2-(2-hydroxy-4-methoxyphenyl)acetic acid (2c);
2-(3-hydroxy-[1,1′-biphenyl]-4-yl)acetic acid (2e);
2-(2-hydroxy-4-iodophenyl)acetic acid (2g);
2-(4-fluoro-2-hydroxyphenyl)acetic acid (2i);
2-(3-hydroxynaphthalen-2-yl)acetic acid (2q);
2-(2-hydroxyphenyl)-2-phenylacetic acid (2u);
2-(3-hydroxydibenzo[b,d]furan-2-yl)acetic acid (2aa);
2-(4-acetamido-2-hydroxyphenyl)acetic acid (2ah); and
(S)-2-(3-hydroxy-6-methoxynaphthalen-2-yl)propanoic acid (2aj).

Embodiment 76. The method of Embodiment 14, wherein 1 mmol benzoic acid of Formula (3) is treated with 4 mol % 2-methyl-2-(6-oxo-1,6-dihydropyridin-2-yl)propanoic acid, 2 mol % Pd(OAc)₂, 3.5 equiv. H₂O₂, and 1.5 equiv. K₂HPO₄·3H₂O in CH₃CN at r.t. for approximately 24 h to form the hydroxylated benzoic acid product of Formula (4).

Embodiment 77. The method of embodiment 76, wherein the hydroxylated benzoic acetic acid product of Formula (4) is selected from the group consisting of:
2-hydroxybenzoic acid (4a);
2-hydroxy-4-methylbenzoic acid (4b);
2-hydroxy-5-methylbenzoic acid (4c);
2-hydroxy-6-methylbenzoic acid (4d);
4-hydroxy-[1,1′-biphenyl]-3-carboxylic acid (4e);
3-hydroxy-[1,1′-biphenyl]-2-carboxylic acid (4f);
2-hydroxy-1-naphthoic acid (4g);
2-hydroxy-4-methoxy-6-methylbenzoic acid (4i);
4-hydroxy-2,3-dihydrobenzofuran-5-carboxylic (4j);
6-hydroxy-2,3-dihydrobenzofuran-5-carboxylic acid (4j′); and
7-hydroxychromane-6-carboxylic acid (4k).

Embodiment 78. The method of Embodiment 14, wherein 1 mmol benzoic acid of Formula (3) is treated with 4 mol % 2-methyl-2-(6-oxo-1,6-dihydropyridin-2-yl)propanoic acid, 2 mol % Pd(OAc)₂, 3.5 equiv. H₂O₂, and 1.5 equiv. K₂HPO₄·3H₂O in CH₃CN at 60° C. for approximately 24 h to form the hydroxylated benzoic acid product of Formula (4).

Embodiment 79. The method of embodiment 78, wherein the hydroxylated benzoic acetic acid product of Formula (4) is selected from the group consisting of:
2-hydroxy-5-(trifluoromethyl)benzoic acid (4h); and
2′,4′-difluoro-4-hydroxy-[1,1′-biphenyl]-3-carboxylic acid (4l).

Embodiment 80. The method of Embodiment 14, wherein 1 mmol benzoic acid of Formula (3) is treated with 10 mol % 2-methyl-2-(6-oxo-1,6-dihydropyridin-2-yl)propanoic acid, 5 mol % Pd(OAc)₂, 3.5 equiv. H₂O₂, and 1.5 equiv. CsOAc in DMA at 60° C. for approximately 24 h to form the hydroxylated benzoic acid product of Formula (4).

Embodiment 81. The method of embodiment 80, wherein the hydroxylated benzoic acetic acid product of Formula (4) is selected from the group consisting of:
2,4-difluoro-6-hydroxybenzoic acid (4m);
4,5-difluoro-2-hydroxybenzoic acid (4n);
3,5-difluoro-2-hydroxybenzoic acid (4o);
2-fluoro-6-hydroxybenzoic acid (4p);
2-hydroxy-4-nitrobenzoic acid (4q);
4-acetyl-2-hydroxybenzoic acid (4r);
2-hydroxy-4-methoxybenzoic acid (4s);
2-hydroxy-6-(4-methylbenzoyl)benzoic acid (4u); and
2-((3-chloro-2-methylphenyl)amino)-6-hydroxybenzoic acid (4v).

Embodiment 82. The method of Embodiment 14, wherein 1 mmol benzoic acid of Formula (3) is treated with 10 mol % 2-methyl-2-(6-oxo-1,6-dihydropyridin-2-yl)propanoic acid, 5 mol % Pd(OAc)₂, 3.5 equiv. H₂O₂, and 1.5 equiv. K₂HPO₄·3H₂O in DMA at 60° C. for approximately 48 h to form the hydroxylated benzoic acid product of Formula (4).

Embodiment 83. The method of embodiment 82, wherein the hydroxylated benzoic acetic acid product of Formula (4) is 2-bromo-4-fluoro-6-hydroxybenzoic acid (4t).

Embodiment 84. The method of Embodiment 14, wherein 0.5 mmol benzoic acid of Formula (3) is treated with 10 mol % 2-methyl-2-(6-oxo-1,6-dihydropyridin-2-yl)propanoic acid, 5 mol % Pd(OAc)₂, 3.5 equiv. H₂O₂, and 1.5 equiv. K₂HPO₄·3H₂O in DMA at r.t. for approximately 24 h to form the hydroxylated benzoic acid product of Formula (4).

Embodiment 85. The method of embodiment 84, wherein the hydroxylated benzoic acetic acid product of Formula (4) is (8R,9S,13S,14S)-2-hydroxy-13-methyl-17-oxo-7,8,9,11,12,13,14,15,16,17-decahydro-6H-cyclopenta[a]phenanthrene-3-carboxylic acid (4w).

Embodiment 86. A method of preparing trifluorinated coumestan and pterocarpene according to the following reaction scheme:

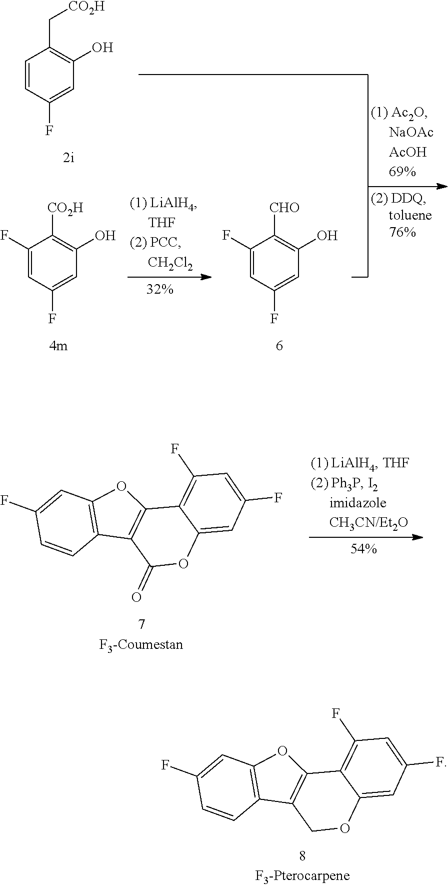 [SMILES: O=C(O)Cc1ccc(F)cc1O ; O=C(O)c1c(O)cc(F)cc1F ; O=Cc1c(O)cc(F)cc1F ; O=c1oc2cc(F)cc(F)c2c2oc3cc(F)ccc3c12 ; Fc1cc(F)c2c(c1)OCc1c-2oc2cc(F)ccc12]

Embodiment 87. A method of hydroxylating ibuprofen according to the following reaction scheme:

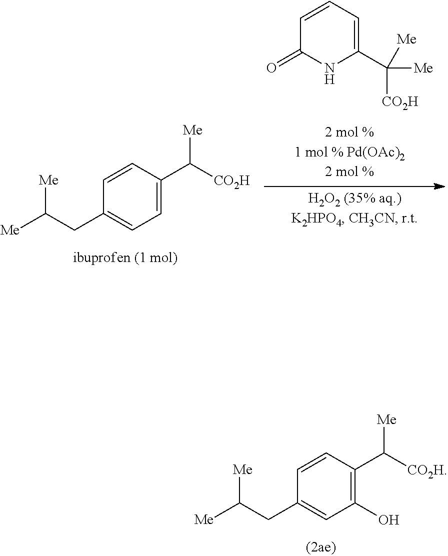 [SMILES: CC(C)(C(=O)O)c1cccc(=O)[nH]1 ; CC(C)Cc1ccc(C(C)C(=O)O)cc1 ; CC(C)Cc1ccc(C(C)C(=O)O)c(O)c1]

Embodiment 88. Any method of C—H hydroxylation of benzoic or phenylacetic acids via treating a benzoic or phenylacetic acid with bifunctional bidentate pyridone-carboxylic acid ligand 2-methyl-2-(6-oxo-1,6-dihydropyridin-2-yl)propanoic acid in aqueous hydrogen peroxide in the presence of a Pd(OAc)₂ and a base, derivatization of the resulting hydroxylation products, synthesis of polyfluorinated natural products coumestan or pterocarpene from phenol building blocks, and hydroxylation of ibuprofen using this methodology, as disclosed herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “˜˜˜˜” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—□—C(—OH)═CH—), amide/imidic acid (—C(═O)—NH—□—C(—OH)═N—) and amidine (—C(═NR)—NH—□—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (Cs), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR'S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂,—SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), 0-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Information

Hydrogen peroxide (35 wt. % in H₂O) was purchased from Acros. Solvents were obtained from Sigma-Aldrich, Alfa-Aesar, and Acros, and used directly without further purification. Other reagents were purchased at the highest commercial quality and used without further purification, unless otherwise stated. Analytical thin layer chromatography was performed on 0.25 mm silica gel 60-F254 or Merck pre-coated aluminium-backed silica gel F254 plates. ¹H NMR spectra were recorded on Bruker AMX-400 or Bruker DRX-600 instruments. The following abbreviations (or combinations thereof) were used to explain multiplicities: s=singlet, d=doublet, t=triplet, q=quartet, m=multiplet, br=broad. Coupling constants, J, were reported in Hertz unit (Hz). ¹³C NMR spectra were recorded on Bruker DRX-600 or JEOL instruments (100 MHz) and were fully decoupled by broad band proton decoupling. ¹⁹F NMR Spectra were recorded on Bruker AMX-399 spectrometer (376 MHz) or JEOL-400 (376 MHz) and were fully decoupled by broad band proton decoupling. Chemical shifts were referenced to the appropriate residual solvent peaks. Column chromatography was carried out automated using Biotage Isolera One with Biotage SNAP Ultra Column. Automated reversed-phase chromatography was carried out using Biotage Isolera One with Biotage SNAP Samplet (C18). High-resolution mass spectra (HRMS) were recorded on an Agilent Mass spectrometer using ESI-TOF (electrospray ionization-time of flight).

### Experimental Section for C—H Hydroxylation

### 2.1. Preparation of Carboxylic Acids Substrates

Carboxylic acid substrates 1aa, 3e were obtained from the Bristol-Myers Squibb compounds collection. Substrates 1ak¹, 3w² were prepared following the reported procedures. Substrate 1aj were prepared with the following procedure. Other substrates are commercially available.

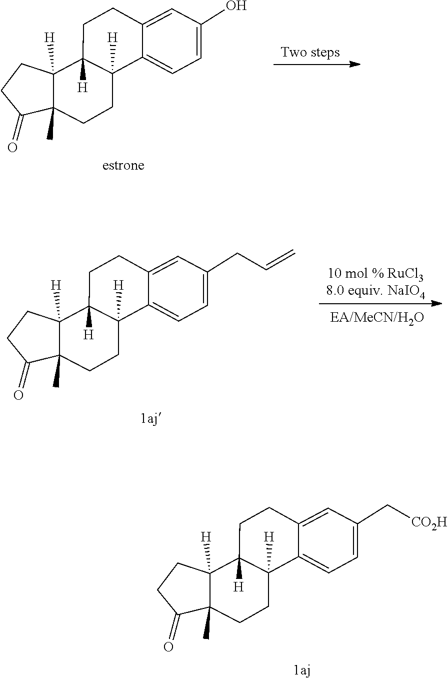

To a solution of 1aj′ (294 mg, 1.0 mmol) (compound 1aj′ was prepared by the known literature³) in EtOAc-MeCN—H₂O 2:2:3 (7 mL) was added RuCl₃ (20.6 mg, 0.1 mmol, 10 mol %). The mixture was stirred and NaIO₄ (1.711 g, 8.0 mmol, 8.0 equiv.) was added in two portions. The reaction solution was stirred vigorously for an additional 6 h. Upon completion, the mixture was diluted with H₂O and extracted with EtOAc. The combined organic phases were dried, filtered, and evaporated under reduced pressure. The residue was subjected to flash chromatography (Hexane-EtOAc 2:1 with 1% AcOH, v/v) to give 1aj as a white solid (221.5 mg, 71%).

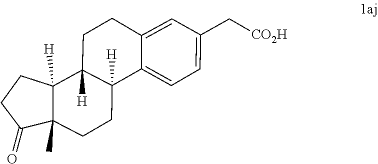

¹H NMR (600 MHz, Chloroform-d) δ 7.26 (d, J=8.0 Hz, 1H), 7.08 (dd, J=8.0, 2.0 Hz, 1H), 7.03 (d, J=2.0 Hz, 1H), 3.59 (s, 2H), 2.93-2.90 (m, 2H), 2.58-2.47 (m, 1H), 2.46-2.38 (m, 1H), 2.33-2.25 (m, 1H), 2.21-2.13 (m, 1H), 2.09-1.94 (m, 3H), 1.68-1.59 (m, 2H), 1.56-1.39 (m, 4H), 0.91 (s, 3H). ¹³C NMR (151 MHz, Chloroform-d) δ 221.42, 177.70, 138.88, 136.87, 130.84, 130.00, 126.81, 125.74, 50.51, 48.07, 44.31, 40.63, 38.10, 35.92, 31.59, 29.34, 26.49, 25.70, 21.63, 13.87. HRMS (ESI-TOF) Calcd for C₂₀H₂₃O₃ [M−H]⁻: 311.1647; found: 311.1647.

### 2.2. Preparation of Bidentate Pyridine-Carboxylic Acid Ligand

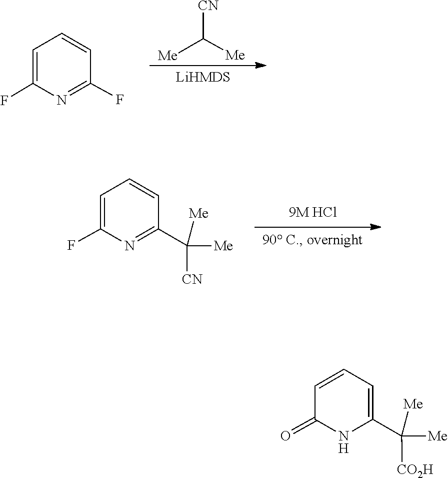 [SMILES: Fc1cccc(F)n1 ; CC(C)C#N ; CC(C)(C#N)c1cccc(F)n1 ; CC(C)(C(=O)O)c1cccc(=O)[nH]1]

Step 1: A solution of LiHMDS (1.0 equiv.) in toluene was added to a solution of 2,6-difluoropyridine (1.5 equiv.) and isobutyronitrile (1.0 equiv.) in toluene at room temperature. The resulting mixture was heated to 80° C. and stirred for overnight. After cooling the mixture to the room temperature, water was added to quench the reaction and toluene was removed under reduced pressure. The resulting mixture was extracted with ethyl acetate several times and combined organic layers were dried over Na₂SO₄ and concentrated to afford the product. In most cases, product was pure enough for the next step. If necessary, column chromatography was used for the purification.

Step 2: The 2-(6-fluoropyridin-2-yl)-2-methylpropanenitrile was weighed to the round bottom flask and was added 9M HCl aq. to make 0.3M solution. The resulting mixture was heated to 90° C. and stirred for overnight. The reaction progress can be monitored through LCMS by consumption of the starting materials. After completion, the solution was concentrated under reduced pressure then diluted with H₂O. 4M NaOH solution was added until the pH reaches 2-3. The resulting aqueous solution was extracted with CHCl₃/IPA (3:1) several times and combined organic layers were dried and concentrated to afford the product. If the ligand is not pure in this stage, residue was purified by column chromatography on silica gel using DCM/MeOH (20:1 with acetic acid) solution as eluent.

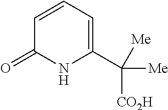 [SMILES: CC(C)(C(=O)O)c1cccc(=O)[nH]1]

### 2-methyl-2-(6-oxo-1,6-dihydropyridin-2-yl) propanoic acid (L4)

¹H NMR (600 MHz, Chloroform-d) δ 7.54 (dd, J=9.1, 7.2 Hz, 1H), 6.56 (dd, J=9.1, 0.9 Hz, 1H), 6.40 (dd, J=7.2, 0.9 Hz, 1H), 1.64 (s, 6H). ¹³C NMR (151 MHz, Chloroform-d) δ 179.15, 165.25, 151.36, 142.81, 118.10, 105.13, 46.17, 25.45. HRMS (ESI-TOF) Calcd for C₉H₁₂O₃ [M+H]⁺: 182.0812; found: 182.0807.

### 2.3. Optimization of the C—H Hydroxylation

TABLE 2
Solvent Effect

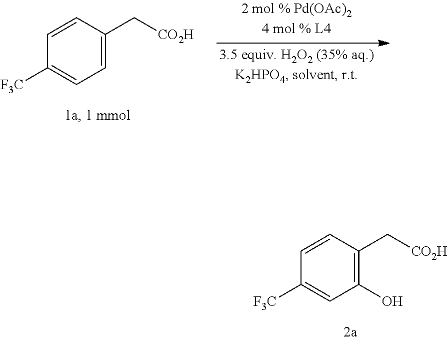 [SMILES: O=C(O)Cc1ccc(C(F)(F)F)cc1 ; O=C(O)Cc1ccc(C(F)(F)F)cc1O]

| Entry | Solvent | Yield (%) |
|---|---|---|
| 1 | DMA | 65 |
| 2 | DMF | 53 |
| 3 | THF | 16 |
| 4 | NMP | 46 |
| 5 | DMSO | <2 |
| 6 | HFIP | <2 |
| 7 | CH3CN | 86 |
| 8 | Acetone | 50 |
| 9 | t-Amyl-OH | 37 |
| 10 | DCE | 35 |

The reaction was performed with carboxylic acid 1a (1 mmol), Pd(OAc)₂ (0.02 mmol), ligand (0.04 mmol), H₂O₂ (35% aqueous solution, 3.5 mmol), and K₂HPO₄ (1.5 mmol) in solvent (3.0 mL) at room temperature for 24 h. Determined by ¹H NMR yield using CH₃NO₂ as the internal standard.

TABLE 3
Base Effect

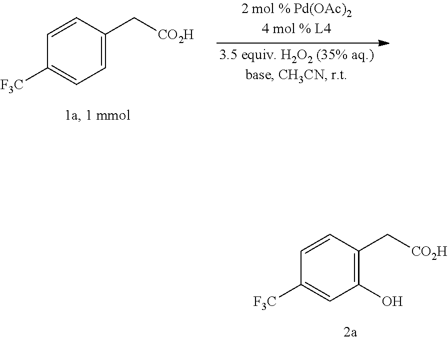 [SMILES: O=C(O)Cc1ccc(C(F)(F)F)cc1 ; O=C(O)Cc1ccc(C(F)(F)F)cc1O]

| Entry | Base | Solvent | Yield (%) |
|---|---|---|---|
| 1 | K₂HPO₄ | CH₃CN | 86 |
| 2 | K₃PO₄ | CH₃CN | <2 |
| 3 | KH₂PO₄ | CH₃CN | <2 |
| 4 | K₂CO₃ | CH₃CN | <2 |
| 5 | KHCO₃ | CH₃CN | <2 |
| 6 | CsOAc | CH₃CN | 60 |
| 7 | NaOAc | CH₃CN | 55 |
| 8 | KOAC | CH₃CN | 58 |
| 9 | Na₃PO₄ | CH₃CN | <2 |
| 10 | / | CH₃CN | 8 |
| 11 | K₂HPO₄ | DMA | 65 |
| 12 | KHCO₃ | DMA | 80 |
| 13 | K₂CO₃ | DMA | <2 |
| 14 | K₃PO₄ | DMA | 17 |
| 15 | NaOAc | DMA | 42 |
| 16 | Na₂HPO₄ | DMA | 25 |
| 17 | KH₂PO₄ | DMA | 5 |
| 18 | KOH | DMA | <2 |

The reaction was performed with carboxylic acid 1a (1 mmol), Pd(OAc)₂ (0.02 mmol), ligand (0.04 mmol), H₂O₂ (35% aqueous solution, 3.5 mmol), and base (1.5 mmol) in solvent(3.0 mL) at room temperature for 24 h. Determined by ¹H NMR yield using CH₃NO₂ as the internal standard.

TABLE 4
Control experiments

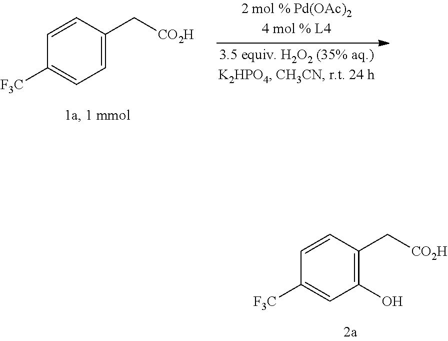 [SMILES: O=C(O)Cc1ccc(C(F)(F)F)cc1 ; O=C(O)Cc1ccc(C(F)(F)F)cc1O]

| Entry | Conditions | Yield (%) |
|---|---|---|
| 1 | / | 86 |
| 2 | w/o Pd(OAc)₂ | 0 |
| 3 | w/o ligand | 0 |
| 4 | w/o H₂O₂ | 0 |
| 5 | w/o K₂HPO₄ | 8 |
| 6 | 1 mol % Pd(OAc)₂ | 76 |
| 7 | 0.5 mol % Pd(OAc)₂ | 46 |
| 8 | 0.1 mol % Pd(OAc)₂ | 5 |
| 9 | 6 h | 44 |
| 10 | 12 h | 80 |

The reaction was performed with carboxylic acid 1a (1 mmol), Pd(OAc)₂ (0.02 mmol), ligand (0.04 mmol), H₂O₂ (35% aqueous solution, 3.5 mmol), and K₂HPO₄ (1.5 mmol) in CH₃CN (3.0 mL) at room temperature for 24 h. Determined by ¹H NMR yield using CH₃NO₂ as the internal standard.

TABLE 5
Optimization using 2,4-difluorobenzoic acid as a substrate

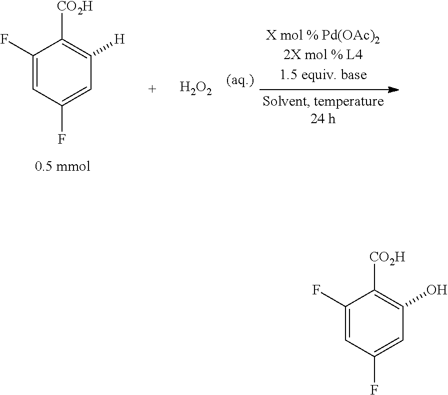

| Pd loading | Solvent | Base | Temp. | Yield |
|---|---|---|---|---|
| 2% | ACN | K₂HPO₄ | r.t. | No product |
| 2% | ACN | K₂HPO₄•3H₂O | r.t. | No product |
| 5% | ACN | K₂HPO₄•3H₂O | r.t. | trace |
| 5% | ACN | K₂HPO₄•3H₂O | 60° C. | 20% |
| 5% | DMA | K₂HPO₄•3H₂O | 60° C. | 33% |
| 5% | DMA | KHCO₃ | 60° C. | 64% |
| 5% | DMA | NaOAc | 60° C. | 70% |
| 5% | DMA | KOAC | 60° C. | 25% |
| 5% | DMA | CsOAc | 60° C. | 78% |

The reaction was performed with carboxylic acid 1a (0.5 mmol), Pd(OAc)₂ (0.01 mmol), ligand (0.02 mmol), H₂O₂(35% aqueous solution, 1.75 mmol), and base (0.75 mmol) in CH₃CN or DMA (1.5 mL) for 24 h. Determined by ¹H NMR yield using CH₃NO₂ as the internal standard.

### 2.4. General Procedures and Characterisation Data

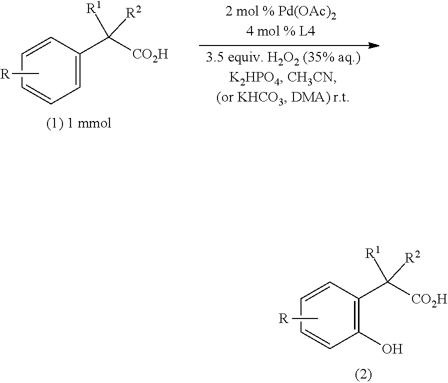

### General Procedure for C(sp²)-H hydroxylation of phenylacetic acids

Pd(OAc)₂ (4.5 mg, 0.02 mmol, 2 mol %), L4 (7.3 mg, 0.04 mmol, 4 mol %), carboxylic acid 1 (1.0 mmol), and K₂HPO₄ (260.0 mg, 1.5 mmol, 1.5 equiv.) were weighed and placed in an 8 mL vial. Then, CH₃CN (3.0 mL) was added and stirred for 10 min, followed by the addition of H₂O₂(35% aq., 300 μL, 3.5 equiv.). The vial was sealed with a screw cap and stirred at ambient temperature for 24 h (typically ran at 25° C. unless otherwise noted). Upon completion, the reaction was diluted with methanol and acidified with 0.3 mL formic acid. The solution was filtered through a pad of Celite and washed with methanol then concentrated under vacuum (The organic phase should be tested by the potassium iodide starch test paper before concentration, and quenched with Na₂S₂O₃ aq. solution if necessary). The crude mixture was purified by flash chromatography (Hexane/EtOAc or DCM/MeOH with 1% AcOH, v/v).

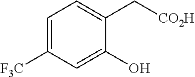 [SMILES: O=C(O)Cc1ccc(C(F)(F)F)cc1O]

### 2-(2-hydroxy-4-(trifluoromethyl)phenyl)acetic acid (2a)

Substrate 1a was hydroxylated following general procedure, the hydroxylated product 2a was obtained as a greyish white solid (175 mg, 80%), ¹H NMR (600 MHz, Methanol-d₄) δ 7.30 (d, J=7.8 Hz, 1H), 7.06 (d, J=7.9 Hz, 1H), 7.03 (s, 1H), 3.65 (s, 2H). ¹³C NMR (151 MHz, Methanol-d₄) δ 175.27, 157.32, 132.82, 131.44 (q, J=32.2 Hz), 127.45, 125.60 (q, J=271.4 Hz), 116.75 (q, J=3.9 Hz), 112.20 (q, J=3.8 Hz), 36.30. ¹⁹F NMR (376 MHz, Methanol-d₄) δ −66.76. HRMS (ESI-TOF) Calcd for C₉H₆F₃O₃[M−H]⁻: 219.0269; found: 219.0270.

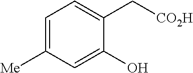 [SMILES: Cc1ccc(CC(=O)O)c(O)c1]

### 2-(2-hydroxy-4-methylphenyl)acetic acid (2b)

Substrate 1b was hydroxylated following general procedure, the hydroxylated product 2b was obtained as a white solid (145 mg, 87%), ¹H NMR (600 MHz, Methanol-d₄) δ 6.97 (d, J=7.5 Hz, 1H), 6.63-6.56 (m, 2H), 3.53 (s, 2H), 2.24 (s, 3H). ¹³C NMR (151 MHz, Methanol-d₄) δ 176.42, 156.54, 139.24, 131.83, 121.14, 119.76, 116.58, 36.18, 21.23. HRMS (ESI-TOF) Calcd for C₉H₉O₃ [M−H]⁻: 165.0552; found: 165.0552.

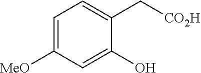 [SMILES: COc1ccc(CC(=O)O)c(O)c1]

### 2-(2-hydroxy-4-methoxyphenyl)acetic acid (2c)

Substrate 1c was hydroxylated following general procedure with KHCO₃ (200.2 mg, 2.0 mmol, 2.0 equiv.) and DMA (3.0 mL) instead of CH₃CN and K₂HPO₄, the hydroxylated product 2c was obtained as a white solid (132 mg, 73%), ¹H NMR (600 MHz, Methanol-d₄) δ 6.99 (d, J=8.2 Hz, 1H), 6.40-6.34 (m, 2H), 3.72 (s, 3H), 3.50 (s, 2H). ¹³C NMR (151 MHz, Methanol-d₄) δ 176.61, 161.41, 157.55, 132.45, 115.27, 105.62, 102.22, 55.59, 35.87. HRMS (ESI-TOF) Calcd for C₉H₉O₄ [M−H]⁻: 181.0501; found: 181.0502.

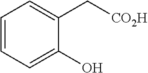 [SMILES: O=C(O)Cc1ccccc1O]

### 2-(2-hydroxyphenyl)acetic acid (2d)

Substrate 1d was hydroxylated following general procedure, the hydroxylated product 2d was obtained as a white solid (129 mg, 85%), ¹H NMR (600 MHz, Methanol-d₄) δ 7.14-7.04 (m, 2H), 6.78-6.75 (m, 2H), 3.58 (s, 2H). ¹³C NMR (151 MHz, Methanol-d₄) δ 176.22, 156.77, 132.07, 129.25, 122.84, 120.41, 115.88, 36.53. HRMS (ESI-TOF) Calcd for C₈H₇O₃[M−H]⁻: 151.0395; found: 151.0393.

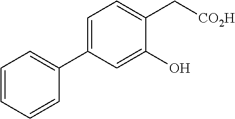 [SMILES: O=C(O)Cc1ccc(-c2ccccc2)cc1O]

### 2-(3-hydroxy-[1,1′-biphenyl]-4-yl)acetic acid (2e)

Substrate 1e was hydroxylated following general procedure with KHCO₃ (200.2 mg, 2.0 mmol, 1.5 equiv.) and DMA (3.0 mL) instead of CH₃CN and K₂HPO₄, the hydroxylated product 2e was obtained as an ivory solid (179 mg, 79%), ¹H NMR (600 MHz, Methanol-d₄) δ 7.58-7.52 (m, 2H), 7.42-7.36 (m, 2H), 7.32-7.25 (m, 1H), 7.18 (d, J=7.7 Hz, 1H), 7.07-7.01 (m, 2H), 3.63 (s, 2H). ¹³C NMR (151 MHz, Methanol-d₄) δ 176.16, 157.06, 142.78, 142.32, 132.48, 129.71, 128.20, 127.80, 122.00, 119.09, 114.38, 36.26. HRMS (ESI-TOF) Calcd for C₁₄H₁₁O₃[M−H]⁻: 227.0708; found: 227.0714.

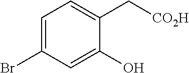 [SMILES: O=C(O)Cc1ccc(Br)cc1O]

### 2-(4-bromo-2-hydroxyphenyl)acetic acid (2f)

Substrate if was hydroxylated following general procedure, the hydroxylated product 2f was obtained as white solid (200 mg, 87%), ¹H NMR (600 MHz, Methanol-d₄) δ 7.03 (d, J=8.0 Hz, 1H), 6.95 (d, J=2.0 Hz, 1H), 6.92 (dd, J=8.0, 2.0 Hz, 1H), 3.54 (s, 2H). ¹³C NMR (151 MHz, Methanol-d₄) δ 175.56, 157.93, 133.46, 123.22, 122.44, 121.87, 118.80, 35.97. HRMS (ESI-TOF) Calcd for C₈H₆BrO₃ [M−H]⁻: 228.9500; found: 228.9503.

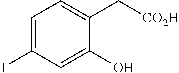 [SMILES: O=C(O)Cc1ccc(I)cc1O]

### 2-(2-hydroxy-4-iodophenyl)acetic acid (2g)

Substrate 1g was hydroxylated following general procedure with KHCO₃ (200.2 mg, 2.0 mmol, 2.0 equiv.) and DMA (3.0 mL) instead of CH₃CN and K₂HPO₄, the hydroxylated product 2g was obtained as a white solid (184 mg, 66%), ¹H NMR (600 MHz, Methanol-d₄) δ 7.14 (d, J=1.7 Hz, 1H), 7.11 (dd, J=7.9, 1.7 Hz, 1H), 6.87 (d, J=7.9 Hz, 1H), 3.53 (s, 2H). ¹³C NMR (151 MHz, Methanol-d₄) δ 175.55, 157.81, 133.68, 129.48, 124.79, 123.05, 92.89, 36.07. HRMS (ESI-TOF) Calcd for C₈H₆ClO₃ [M−H]⁻: 276.9362; found: 276.9365.

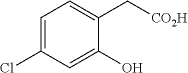 [SMILES: O=C(O)Cc1ccc(Cl)cc1O]

### 2-(4-chloro-2-hydroxyphenyl)acetic acid (2h)

Substrate 1h was hydroxylated following general procedure, the hydroxylated product 2h was obtained as a white solid (150 mg, 81%), ¹H NMR (600 MHz, Methanol-d₄) δ 7.08 (d, J=8.0 Hz, 1H), 6.79 (d, J=2.1 Hz, 1H), 6.77 (dd, J=8.0, 2.2 Hz, 1H), 3.55 (s, 2H). ¹³C NMR (151 MHz, Methanol-d₄) δ 175.67, 157.75, 134.16, 133.10, 121.95, 120.21, 115.87, 35.90. HRMS (ESI-TOF) Calcd for C₈H₆ClO₃ [M−H]⁻: 185.0005; found: 185.0005.

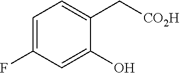 [SMILES: O=C(O)Cc1ccc(F)cc1O]

### 2-(4-fluoro-2-hydroxyphenyl)acetic acid (2i)

Substrate 1i was hydroxylated following general procedure with KHCO₃ (200.2 mg, 2.0 mmol, 2.0 equiv.) and DMA (3.0 mL) instead of CH₃CN and K₂HPO₄, the hydroxylated product 2i was obtained as a white solid (123 mg, 72%), ¹H NMR (600 MHz, Methanol-d₄) δ 7.09 (dd, J=8.3, 6.7 Hz, 1H), 6.56-6.44 (m, 2H), 3.54 (s, 2H). ¹³C NMR (151 MHz, Methanol-d₄) δ 176.04, 164.05 (d, J=242.5 Hz), 158.05 (d, J=11.0 Hz), 132.89 (d, J=10.1 Hz), 119.03 (d, J=3.2 Hz), 106.55 (d, J=21.4 Hz), 103.04 (d, J=24.3 Hz), 35.82. ¹⁹F NMR (376 MHz, Methanol-d₄) δ −119.34. HRMS (ESI-TOF) Calcd for C₈H₆FO₃ [M−H]⁻: 169.0301; found: 169.0306.

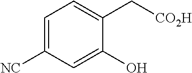 [SMILES: N#Cc1ccc(CC(=O)O)c(O)c1]

### 2-(4-cyano-2-hydroxyphenyl)acetic acid (2j)

Substrate 1j was hydroxylated following general procedure at 60° C., the hydroxylated product 2j was obtained as a yellow solid (131 mg, 74%), ¹H NMR (600 MHz, Methanol-d₄) δ 7.30 (d, J=7.7 Hz, 1H), 7.14 (dd, J=7.8, 1.6 Hz, 1H), 7.05 (d, J=1.6 Hz, 1H), 3.65 (s, 2H). ¹³C NMR (151 MHz, Methanol-d₄) δ 174.80, 157.51, 133.34, 129.47, 124.14, 119.78, 118.45, 112.48, 36.44. HRMS (ESI-TOF) Calcd for C₉H₆NO₃ [M−H]⁻: 176.0348; found: 176.0345.

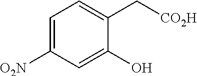 [SMILES: O=C(O)Cc1ccc([N+](=O)[O-])cc1O]

### 2-(2-hydroxy-4-nitrophenyl)acetic acid (2k)

Substrate 1k was hydroxylated following general procedure at 60° C., the hydroxylated product 2k was obtained as an orange solid (138 mg, 70%), ¹H NMR (600 MHz, Methanol-d₄) δ 7.66 (dd, J=8.3, 2.3 Hz, 1H), 7.62 (d, J=2.3 Hz, 1H), 7.35 (d, J=8.3 Hz, 1H), 3.69 (s, 2H). ¹³C NMR (151 MHz, Methanol-d₄) δ 174.66, 157.60, 149.32, 132.74, 130.99, 115.08, 110.07, 36.33. HRMS (ESI-TOF) Calcd for C₈H₆NO₅ [M−H]⁻: 196.0246; found: 196.0247.

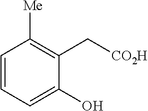 [SMILES: Cc1cccc(O)c1CC(=O)O]

### 2-(2-hydroxy-6-methylphenyl)acetic acid (2l)

Substrate 1l was hydroxylated following general procedure, the hydroxylated product 21 was obtained as an ivory solid (143 mg, 86%), ¹H NMR (600 MHz, Methanol-d₄) δ 6.95 (t, J=7.8 Hz, 1H), 6.73-6.59 (m, 2H), 3.67 (s, 2H), 2.24 (s, 3H). ¹³C NMR (151 MHz, Methanol-d₄) δ 176.07, 156.75, 139.49, 128.48, 122.21, 121.57, 113.48, 32.42, 19.78. HRMS (ESI-TOF) Calcd for C₉H₉O₃ [M−H]⁻: 165.0552; found: 165.0550.

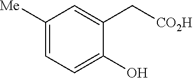 [SMILES: Cc1ccc(O)c(CC(=O)O)c1]

### 2-(2-hydroxy-5-methylphenyl)acetic acid (2m)

Substrate 1m was hydroxylated following general procedure, the hydroxylated product 2m was obtained as a white solid (131 mg, 79%), ¹H NMR (600 MHz, Methanol-d₄) δ 6.92 (d, J=2.2 Hz, 1H), 6.88 (dd, J=8.1, 2.3 Hz, 1H), 6.67 (d, J=8.1 Hz, 1H), 3.54 (s, 2H), 2.21 (s, 3H). ¹³C NMR (151 MHz, Methanol-d₄) δ 176.31, 154.34, 132.56, 129.59, 129.54, 122.47, 115.80, 36.52, 20.50. HRMS (ESI-TOF) Calcd for C₉H₉O₃ [M−H]⁻: 165.0552; found: 165.0554.

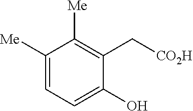 [SMILES: Cc1ccc(O)c(CC(=O)O)c1C]

### 2-(6-hydroxy-2,3-dimethylphenyl)acetic acid (2n)

Substrate in was hydroxylated following general procedure, the hydroxylated product 2n was obtained as an ivory solid (128 mg, 71%), ¹H NMR (600 MHz, Methanol-d₄) δ 6.85 (d, J=8.2 Hz, 1H), 6.55 (d, J=8.2 Hz, 1H), 3.71 (s, 2H), 2.17 (s, 3H), 2.13 (s, 3H), ¹³C NMR (151 MHz, Methanol-d₄) δ 176.35, 154.70, 137.49, 129.84, 128.32, 121.50, 112.93, 32.70, 20.11, 15.89. HRMS (ESI-TOF) Calcd for C₁₀H₁₁O₃[M−H]⁻: 179.0708; found: 179.0710.

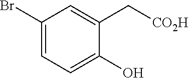 [SMILES: O=C(O)Cc1cc(Br)ccc1O]

### 2-(5-bromo-2-hydroxyphenyl)acetic acid (2o)

Substrate to was hydroxylated following general procedure, the hydroxylated product 2o was obtained as a white solid (190 mg, 83%), ¹H NMR (600 MHz, Methanol-d₄) δ 7.26 (d, J=2.6 Hz, 1H), 7.19 (dd, J=8.6, 2.6 Hz, 1H), 6.70 (d, J=8.6 Hz, 1H), 3.56 (s, 2H). ¹³C NMR (151 MHz, Methanol-d₄) δ 175.43, 156.20, 134.66, 131.88, 125.46, 117.54, 111.74, 36.09. HRMS (ESI-TOF) Calcd for C₈H₆BrO₃ [M−H]⁻: 228.9500; found: 228.9503.

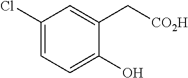 [SMILES: O=C(O)Cc1cc(Cl)ccc1O]

### 2-(5-chloro-2-hydroxyphenyl)acetic acid (2p)

Substrate Ip was hydroxylated following general procedure, the hydroxylated product 2p was obtained as a white solid (158 mg, 85%), ¹H NMR (600 MHz, Methanol-d₄) δ 7.12 (d, J=2.6 Hz, 1H), 7.05 (dd, J=8.6, 2.7 Hz, 1H), 6.74 (d, J=8.6 Hz, 1H), 3.56 (s, 2H). ¹³C NMR (151 MHz, Methanol-d₄) δ 175.46, 155.68, 131.73, 128.86, 124.88, 124.72, 117.03, 36.17. HRMS (ESI-TOF) Calcd for C₈H₆ClO₃ [M−H]⁻: 185.0005; found: 185.0008.

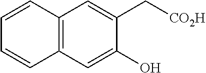 [SMILES: O=C(O)Cc1cc2ccccc2cc1O]

### 2-(3-hydroxynaphthalen-2-yl)acetic acid (2q)

Substrate 1q was hydroxylated following general procedure with KHCO₃ (200.2 mg, 2.0 mmol, 2.0 equiv.) and DMA (3.0 mL) instead of CH₃CN and K₂HPO₄, the hydroxylated product 2q was obtained as a white solid (166 mg, 82%), ¹H NMR (600 MHz, Methanol-d₄) δ 7.68 (d, J=8.2 Hz, 1H), 7.61 (s, 1H), 7.59 (d, J=7.9 Hz, 1H), 7.33 (ddd, J=8.1, 6.8, 1.3 Hz, 1H), 7.23 (ddd, J=8.1, 6.8, 1.2 Hz, 1H), 7.10 (s, 1H), 3.75 (s, 2H). ¹³C NMR (151 MHz, Methanol-d₄) δ 176.03, 155.33, 135.86, 131.05, 129.85, 128.35, 126.81, 126.72, 125.85, 123.94, 109.53, 37.18. HRMS (ESI-TOF) Calcd for C₁₂H₉O₃ [M−H]⁻: 201.0552; found: 201.0552.

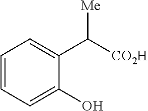 [SMILES: CC(C(=O)O)c1ccccc1O]

### 2-(2-hydroxyphenyl)propanoic acid (2r)

Substrate 1r was hydroxylated following general procedure, the hydroxylated product 2r was obtained as a colourless liquid (146 mg, 88%), ¹H NMR (600 MHz, Methanol-d₄) δ 7.13 (dd, J=7.6, 1.7 Hz, 1H), 7.05 (td, J=7.7, 1.7 Hz, 1H), 6.82-6.74 (m, 2H), 4.00 (q, J=7.2 Hz, 1H), 1.41 (d, J=7.2 Hz, 3H). ¹³C NMR (151 MHz, Methanol-d₄) δ 179.44, 155.96, 129.10, 128.96, 128.83, 120.58, 116.17, 40.74, 17.70. HRMS (ESI-TOF) Calcd for C₉H₉O₃ [M−H]⁻: 165.0552; found: 165.0552.

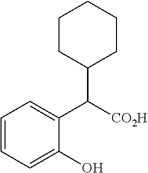 [SMILES: O=C(O)C(c1ccccc1O)C1CCCCC1]

### 2-cyclohexyl-2-(2-hydroxyphenyl)acetic acid (2s)

Substrate is was hydroxylated following general procedure, the hydroxylated product 2s was obtained as a white solid (173 mg, 74%), ¹H NMR (600 MHz, Methanol-d₄) δ 7.27 (dd, J=7.9, 1.6 Hz, 1H), 7.04 (td, J=7.7, 1.6 Hz, 1H), 6.81-6.75 (m, 2H), 3.80 (d, J=10.6 Hz, 1H), 1.98-1.88 (m, 2H), 1.78-1.74 (m, 1H), 1.66-1.60 (m, 2H), 1.36-1.28 (m, 2H), 1.21-1.08 (m, 3H), 0.89-0.82 (m, 1H). ¹³C NMR (151 MHz, Methanol-d₄) δ 178.57, 156.67, 129.74, 128.85, 125.77, 120.61, 116.32, 51.59, 41.75, 33.24, 31.01, 27.53, 27.21. HRMS (ESI-TOF) Calcd for C₁₄H₁₇O₃[M−H]⁻: 233.1178; found: 233.1185.

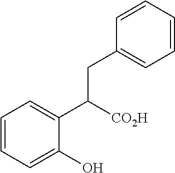 [SMILES: O=C(O)C(Cc1ccccc1)c1ccccc1O]

### 2-(2-hydroxyphenyl)-3-phenylpropanoic acid (2t)

Substrate it was hydroxylated following general procedure, the hydroxylated product 2t was obtained as a white solid (171 mg, 71%), ¹H NMR (600 MHz, Methanol-d₄) δ 7.21-7.10 (m, 6H), 7.05 (td, J=7.6, 1.6 Hz, 1H), 6.78 (dd, J=8.1, 1.4 Hz, 1H), 6.77-6.72 (m, 1H), 4.25 (t, J=7.6 Hz, 1H), 3.27-3.23 (m, 1H), 3.04-2.96 (m, 1H). ¹³C NMR (151 MHz, Methanol-d₄) δ 177.79, 156.10, 141.35, 130.03, 129.53, 129.09, 129.05, 127.09, 127.04, 120.46, 116.23, 48.24, 39.44. HRMS (ESI-TOF) Calcd for C₁₅H₁₃O₃[M−H]⁻: 241.0865; found: 241.0872.

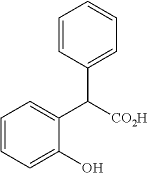 [SMILES: O=C(O)C(c1ccccc1)c1ccccc1O]

### 2-(2-hydroxyphenyl)-2-phenylacetic acid (2u)

Substrate 1u was hydroxylated following general procedure with KHCO₃ (200.2 mg, 2.0 mmol, 2.0 equiv.) and DMA (3.0 mL) instead of CH₃CN and K₂HPO₄, the hydroxylated product 2u was obtained as a white solid (135 mg, 59%), ¹H NMR (600 MHz, Methanol-d₄) δ 7.34-7.27 (m, 4H), 7.26-7.23 (m, 1H), 7.07 (t, J=7.7 Hz, 1H), 6.95 (d, J=7.7 Hz, 1H), 6.80 (d, J=8.1 Hz, 1H), 6.73 (t, J=7.5 Hz, 1H), 5.29 (s, 1H). ¹³C NMR (151 MHz, Methanol-d₄) δ 177.02, 156.23, 139.88, 130.30, 130.12, 129.38, 129.14, 127.96, 127.49, 120.21, 115.82, 52.39. HRMS (ESI-TOF) Calcd for C₁₄H₁₁O₃[M−H]⁻: 227.0708; found: 227.0708.

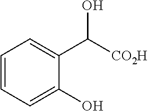 [SMILES: O=C(O)C(O)c1ccccc1O]

### 2-hydroxy-2-(2-hydroxyphenyl)acetic acid (2v)

Substrate 1v was hydroxylated following general procedure, the hydroxylated product 2v was obtained as a as viscous liquid (139 mg, 83%), ¹H NMR (600 MHz, Methanol-d₄) δ 7.34 (d, J=7.7 Hz, 1H), 7.18-7.07 (m, 1H), 6.90-6.73 (m, 2H), 5.39 (s, 1H). ¹³C NMR (151 MHz, MeOD) δ 177.88, 156.25, 130.08, 128.79, 127.72, 120.62, 117.00, 70.52. HRMS (ESI-TOF) Calcd for C₈H₇O₄[M−H]⁻: 167.0344; found: 167.0346.

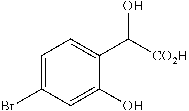 [SMILES: O=C(O)C(O)c1ccc(Br)cc1O]

### 2-(4-bromo-2-hydroxyphenyl)-2-hydroxyacetic (2w)

Substrate 1w was hydroxylated following general procedure at 60° C., the hydroxylated product 2w was obtained as a as viscous liquid (115 mg, 47%), ¹H NMR (400 MHz, Methanol-d₄) δ 7.30 (d, J=8.2 Hz, 1H), 6.98-6.90 (m, 2H), 5.20 (s, 1H). ¹³C NMR (151 MHz, Methanol-d₄) δ 178.80, 157.27, 129.64, 127.99, 123.52, 122.53, 120.38, 70.65. HRMS (ESI-TOF) Calcd for C₈H₆BrO₄ [M−H]⁻: 244.9449; found: 244.9455.

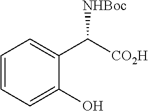 [SMILES: CC(C)(C)OC(=O)N[C@H](C(=O)O)c1ccccc1O]

### (S)-2-((tert-butoxycarbonyl)amino)-2-(2-hydroxyphenyl)acetic (2x)

Substrate 1x was hydroxylated following general procedure, the hydroxylated product 2x was obtained as a yellow solid (155 mg, 58%), ¹H NMR (600 MHz, Methanol-d₄) δ 7.20 (dd, J=7.8, 1.7 Hz, 1H), 7.15-7.10 (m, 1H), 6.83-6.78 (m, 2H), 5.38 (s, 1H), 1.44 (s, 9H). ¹³C NMR (151 MHz, MeOD) δ 175.64, 157.65, 156.42, 130.23, 130.05, 128.51, 125.63, 120.63, 116.69, 80.71, 28.71. HRMS (ESI-TOF) Calcd for C₁₃H₁₆NO₅ [M−H]⁻: 266.1034; found: 266.1034.

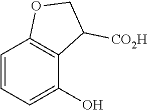 [SMILES: O=C(O)C1COc2cccc(O)c21]

### 4-hydroxy-2,3-dihydrobenzofuran-3-carboxylic acid (2y)

Substrate 1y was hydroxylated following general procedure, the hydroxylated product 2y was obtained as a white solid (135 mg, 75%), ¹H NMR (600 MHz, Methanol-d₄) δ 6.98 (t, J=8.1, 1H), 6.33 (d, J=8.1 Hz, 1H), 6.28 (d, J=7.9 Hz, 1H), 4.70 (dd, J=9.1, 6.0 Hz, 1H), 4.64 (dd, J=9.7, 9.0 Hz, 1H), 4.33 (dd, J=9.7, 6.0 Hz, 1H). ¹³C NMR (151 MHz, Methanol-d₄) δ 176.19, 163.18, 155.97, 131.25, 112.05, 109.13, 102.21, 74.80, 47.04. HRMS (ESI-TOF) Calcd for C₉H₇O₄ [M−H]⁻: 179.0344; found: 179.0347.

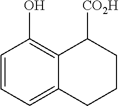 [SMILES: O=C(O)C1CCCc2cccc(O)c21]

### 8-hydroxy-1,2,3,4-tetrahydronaphthalene-1-carboxylic acid (2z)

Substrate 1z was hydroxylated following general procedure, the hydroxylated product 2z was obtained as an ivory solid (123 mg, 64%), ¹H NMR (600 MHz, Methanol-d₄) δ 6.95 (t, J=7.8 Hz, 1H), 6.61-6.55 (m, 2H), 3.80 (dd, J=6.8, 4.9 Hz, 1H), 2.81-2.63 (m, 2H), 2.12-2.06 (m, 1H), 2.04-1.99 (m, 1H), 1.88-1.81 (m, 1H), 1.76-1.69 (m, 1H). ¹³C NMR (151 MHz, Methanol-d₄) δ 180.15, 156.71, 139.85, 128.20, 122.79, 121.15, 112.74, 41.29, 30.44, 28.49, 21.53. HRMS (ESI-TOF) Calcd for C₁₁H₁₁O₃[M−H]⁻: 191.0708; found: 191.0714.

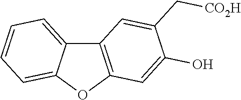 [SMILES: O=C(O)Cc1cc2c(cc1O)oc1ccccc12]

### 2-(3-hydroxydibenzo[b,d]furan-2-yl)acetic acid (2aa)

Substrate 1aa was hydroxylated following general procedure with KHCO₃ (200.2 mg, 2.0 mmol, 2.0 equiv.) and DMA (3.0 mL) instead of CH₃CN and K₂HPO₄, the hydroxylated product 2aa was obtained as a white solid (160 mg, 66%), ¹H NMR (600 MHz, Methanol-d₄) δ 7.81 (dd, J=7.6, 1.4 Hz, 1H), 7.71 (s, 1H), 7.45 (d, J=8.1 Hz, 1H), 7.34-7.28 (m, 1H), 7.25 (td, J=7.5, 1.0 Hz, 1H), 6.98 (s, 1H), 3.72 (s, 2H). ¹³C NMR (151 MHz, Methanol-d₄) δ 176.42, 158.04, 157.48, 157.23, 126.41, 125.84, 123.72, 123.44, 120.51, 119.40, 117.18, 111.97, 98.54, 36.81. HRMS (ESI-TOF) Calcd for C₁₄H₉O₄ [M−H]⁻: 241.0501; found: 241.0503.

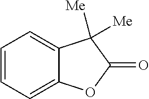 [SMILES: CC1(C)C(=O)Oc2ccccc21]

Compound 2ab easily converts into the corresponding lactone during work-up and purification. The isolated yield is based on the lactone 2ab′ after following the produce below.

Procedure: Upon completion of C—H hydroxylation, 0.5 mL concentrated sulfuric acid was added to the reaction mixture and stirred for 2 h. The solution was quenched with saturated solution of Na₂SO₃ in water. The mixture was filtered through Celite, and the aqueous layer was extracted with ethyl acetate three times. The combined organic layers were dried over anhydrous Na₂SO₄ and concentrated under vacuum. The crude mixture was purified by flash chromatography affording the product as colourless liquid, 130 mg, 80%. (Hexane/EtOAc=10/1, v/v).

### 3,3-dimethylbenzofuran-2(3H)-one (2ab′)

¹H NMR (600 MHz, Chloroform-d) δ 7.38 (td, J=7.8, 1.5 Hz, 1H), 7.31 (dd, J=7.4, 1.4 Hz, 1H), 7.25 (td, J=7.5, 1.0 Hz, 1H), 7.21 (dt, J=7.9, 0.8 Hz, 1H), 1.59 (s, 6H). ¹³C NMR (151 MHz, Chloroform-d) δ 181.07, 152.37, 133.82, 128.66, 124.40, 122.86, 110.96, 43.03, 25.40.

The NMR data matches the reported data⁴.

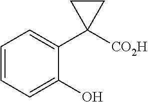 [SMILES: O=C(O)C1(c2ccccc2O)CC1]

### 1-(2-hydroxyphenyl)cyclopropane-1-carboxylic acid (2ac)

Substrate 1ac was hydroxylated following general procedure, the hydroxylated product 2ac was obtained as a colourless oil (139 mg, 78%), ¹H NMR (600 MHz, Methanol-d₄) δ 7.14 (dd, J=7.4, 1.7 Hz, 1H), 7.07 (td, J=7.7, 1.7 Hz, 1H), 6.75 (m, 2H), 1.56 (q, J=4.1 Hz, 2H), 1.11 (q, J=4.0 Hz, 2H). ¹³C NMR (151 MHz, Methanol-d₄) δ 178.73, 158.20, 131.85, 129.33, 127.74, 120.11, 116.03, 25.74, 17.19. HRMS (ESI-TOF) Calcd for C₁₀H₉O₃ [M−H]⁻: 177.0552; found: 177.0550.

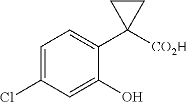 [SMILES: O=C(O)C1(c2ccc(Cl)cc2O)CC1]

### 1-(4-chloro-2-hydroxyphenyl)cyclopropane-1-carboxylic acid (2ad)

Substrate 1ad was hydroxylated following general procedure, the hydroxylated product 2ad was obtained as a yellow solid (119 mg, 56%), ¹H NMR (600 MHz, Methanol-d₄) δ 7.10 (d, J=8.1 Hz, 1H), 6.78 (d, J=2.1 Hz, 1H), 6.75 (dd, J=8.1, 2.1 Hz, 1H), 1.57-1.53 (m, 2H), 1.11-1.07 (m, 2H). ¹³C NMR (151 MHz, MeOD) δ 178.38, 159.25, 134.28, 132.92, 127.02, 119.90, 116.07, 25.43, 17.06. HRMS (ESI-TOF) Calcd for C₁₀H₈ClO₃ [M−H]⁻: 211.0162; found: 211.0157.

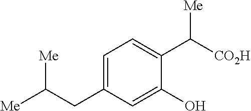 [SMILES: CC(C)Cc1ccc(C(C)C(=O)O)c(O)c1]

### 2-(2-hydroxy-4-isobutylphenyl)propanoic acid (2ae)

Substrate 1ae was hydroxylated following general procedure, the hydroxylated product 2ae was obtained as a white solid (195 mg, 88%), ¹H NMR (600 MHz, Chloroform-d) δ 7.06 (d, J=7.8 Hz, 1H), 6.71 (dd, J=7.7, 1.7 Hz, 1H), 6.67 (d, J=1.7 Hz, 1H), 3.93 (q, J=7.2 Hz, 1H), 2.40 (d, J=7.2 Hz, 2H), 1.85-1.81 (m, 1H), 1.55 (d, J=7.3 Hz, 3H), 0.89 (d, J=6.7 Hz, 6H). ¹³C NMR (151 MHz, Chloroform-d) δ 182.11, 153.79, 143.13, 128.34, 122.83, 122.20, 118.02, 45.08, 40.84, 30.15, 22.55, 16.14. HRMS (ESI-TOF) Calcd for C₁₃H₁₇O₃[M−H]⁻: 221.1178; found: 221.1179.

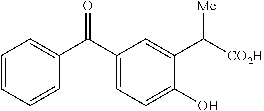 [SMILES: CC(C(=O)O)c1cc(C(=O)c2ccccc2)ccc1O]

### 2-(5-benzoyl-2-hydroxyphenyl)propanoic acid (2af)

Substrate 1af was hydroxylated following general procedure, the hydroxylated product 2af was obtained as a white solid (151 mg, 56%), ¹H NMR (600 MHz, Methanol-d₄) δ 7.75-7.67 (m, 3H), 7.63-7.58 (m, 2H), 7.54-7.48 (m, 2H), 6.90 (d, J=8.4 Hz, 1H), 4.05 (q, J=7.2 Hz, 1H), 1.44 (d, J=7.2 Hz, 3H). ¹³C NMR (151 MHz, Methanol-d₄) δ 197.81, 178.56, 161.20, 139.75, 133.12, 132.49, 132.33, 130.65, 129.88, 129.51, 129.37, 115.69, 40.53, 17.46. HRMS (ESI-TOF) Calcd for C₁₆H₁₃O₄[M−H]⁻: 269.0814; found: 269.0813.

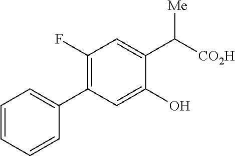 [SMILES: CC(C(=O)O)c1cc(F)c(-c2ccccc2)cc1O]

### 2-(2-fluoro-5-hydroxy-[11,1′-biphenyl]-4-yl)propanoic acid (2ag)

Substrate 1ag was hydroxylated following general procedure, the hydroxylated product 2ag was obtained as a pale-yellow solid (174 mg, 67%), ¹H NMR (600 MHz, Methanol-d₄) δ 7.49 (d, J=7.3 Hz, 2H), 7.41 (t, J=7.8 Hz, 2H), 7.36-7.29 (m, 1H), 6.99 (d, J=11.6 Hz, 1H), 6.85 (d, J=6.8 Hz, 1H), 4.03 (q, J=7.2 Hz, 1H), 1.46 (d, J=7.2 Hz, 3H). ¹³C NMR (151 MHz, Methanol-d₄) δ 178.54, 154.50 (d, J=237.7 Hz), 152.24 (d, J=2.1 Hz), 137.25, 129.95 (d, J=7.2 Hz), 129.86 (d, J=2.9 Hz), 129.40, 128.95 (d, J=14.9 Hz), 128.57, 117.13 (d, J=3.5 Hz), 116.29 (d, J=25.2 Hz), 40.28, 17.62. ¹⁹F NMR (376 MHz, Methanol-d₄) δ −134.88. HRMS (ESI-TOF) Calcd for C₁₅H₁₂FO₃ [M−H]⁻: 259.0770; found: 259.0769.

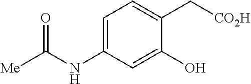 [SMILES: CC(=O)Nc1ccc(CC(=O)O)c(O)c1]

### 2-(4-acetamido-2-hydroxyphenyl)acetic acid (2ah)

Substrate 1ah was hydroxylated following general procedure with KHCO₃ (200.2 mg, 2.0 mmol, 2.0 equiv.) and DMA (3.0 mL) instead of CH₃CN and K₂HPO₄, the hydroxylated product 2ah was obtained as an ivory solid (140 mg, 67%), ¹H NMR (600 MHz, Methanol-d₄) δ 7.25 (d, J=2.1 Hz, 1H), 7.03 (d, J=8.1 Hz, 1H), 6.83 (dd, J=8.1, 2.1 Hz, 1H), 3.53 (s, 2H), 2.08 (s, 3H). ¹³C NMR (151 MHz, Methanol-d₄) δ 176.39, 171.55, 156.92, 139.83, 131.98, 118.82, 112.06, 108.17, 36.29, 23.80. HRMS (ESI-TOF) Calcd for C₁₀H₁₀NO₄ [M−H]⁻: 208.0610; found: 208.0609.

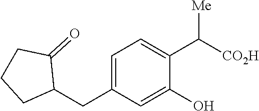 [SMILES: CC(C(=O)O)c1ccc(CC2CCCC2=O)cc1O]

### 2-(2-hydroxy-4-((2-oxocyclopentyl)methyl)phenyl)propanoic acid (2ai)

Substrate 1ai was hydroxylated following general procedure, the hydroxylated product 2ai was obtained as an yellow oil (197 mg, 75%), ¹H NMR (600 MHz, Methanol-d₄) δ 7.04 (d, J=8.3 Hz, 1H), 6.65-6.58 (m, 2H), 3.97 (q, J=7.2 Hz, 1H), 2.98-2.94 (m, 1H), 2.47-2.42 (m, 1H), 2.39-2.25 (m, 2H), 2.12-2.03 (m, 2H), 1.96-1.91 (m, 1H), 1.79-1.70 (m, 1H), 1.60-1.53 (m, 1H), 1.39 (d, J=7.2 Hz, 3H). ¹³C NMR (151 MHz, Methanol-d₄) δ 222.98, 179.31, 155.85, 141.17, 128.87, 126.89, 121.19, 116.56, 52.04, 40.16, 39.03, 36.17, 30.08, 21.45, 17.72. HRMS (ESI-TOF) Calcd for C₁₅H₁₇O₄[M−H]⁻: 261.1127; found: 261.1129.

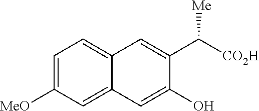 [SMILES: COc1ccc2cc([C@H](C)C(=O)O)c(O)cc2c1]

### (S)-2-(3-hydroxy-6-methoxynaphthalen-2-yl)propanoic acid (2aj)

Substrate 1aj was hydroxylated following general procedure, the hydroxylated product 2aj was obtained as an orange solid (160 mg, 65%) with KHCO₃ (200.2 mg, 2.0 mmol, 2.0 equiv.) and DMA (3.0 mL) instead of CH₃CN and K₂HPO₄, ¹H NMR (600 MHz, Methanol-d₄) δ 7.57 (d, J=8.9 Hz, 1H), 7.53 (s, 1H), 7.03 (s, 1H), 6.97 (d, J=2.5 Hz, 1H), 6.88 (dd, J=8.9, 2.5 Hz, 1H), 4.09 (q, J=7.2 Hz, 1H), 3.84 (s, 3H), 1.52 (d, J=7.2 Hz, 3H). ¹³C NMR (151 MHz, Methanol-d₄) δ 179.32, 159.32, 155.24, 136.73, 129.97, 129.22, 127.84, 125.26, 116.47, 109.27, 105.03, 55.57, 40.99, 17.74. HRMS (ESI-TOF) Calcd for C₁₄H₁₃O₄ [M−H]⁻: 245.0814; found: 245.0817.

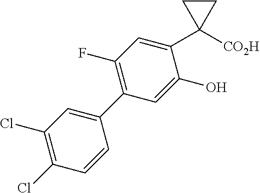 [SMILES: O=C(O)C1(c2cc(F)c(-c3ccc(Cl)c(Cl)c3)cc2O)CC1]

### 1-(3′,4′-dichloro-2-fluoro-5-hydroxy-[1,1′-biphenyl]-4-yl)cyclopropane-1-carboxylic acid (2ak)

Substrate 1ak was hydroxylated following general procedure on 0.5 mmol scale, the hydroxylated product 2ak was obtained as a white solid (126 mg, 74%), ¹H NMR (600 MHz, Methanol-d₄) δ 7.64 (dd, J=2.0, 1.3 Hz, 1H), 7.55 (d, J=8.3 Hz, 1H), 7.42 (dt, J=8.3, 1.6 Hz, 1H), 7.00 (d, J=11.2 Hz, 1H), 6.82 (d, J=6.7 Hz, 1H), 1.62-1.55 (m, 2H), 1.18-1.11 (m, 2H). ¹³C NMR (151 MHz, Methanol-d₄) δ 178.01, 154.83, 153.82 (d, J=238.4 Hz), 137.55, 133.29, 132.52, 131.61 (d, J=3.8 Hz), 131.54, 130.15 (d, J=7.6 Hz), 129.62 (d, J=3.3 Hz), 126.73 (d, J=14.8 Hz), 119.18 (d, J=24.8 Hz), 116.76 (d, J=2.7 Hz), 26.00, 17.09. HRMS (ESI-TOF) Calcd for C₁₆H₁₀Cl₂FO₃ [M−H]⁻: 338.9991; found: 338.9992.

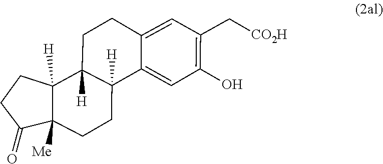

### 2-((8R,9S,13S,14S)-2-hydroxy-13-methyl-17-oxo-7,8,9,11,12,13,14,15,16,17-decahydro-6H-cyclopenta[a]phenanthren-3-yl)acetic acid (2al)

Substrate 1al was hydroxylated following general procedure on 0.5 mmol scale, the hydroxylated product 2al was obtained as a white solid (128 mg, 78%), ¹H NMR (600 MHz, Chloroform-d) δ 6.86 (s, 1H), 6.84 (s, 1H), 3.64 (s, 2H), 2.85-2.79 (m, 2H), 2.55-2.48 (m, 1H), 2.34-2.11 (m, 3H), 2.07-1.92 (m, 3H), 1.66-1.36 (m, 6H), 0.89 (s, 3H). ¹³C NMR (151 MHz, Chloroform-d) δ 221.65, 178.28, 152.52, 141.06, 131.63, 129.12, 118.02, 114.07, 50.59, 48.17, 44.45, 38.11, 36.77, 36.03, 31.66, 28.54, 26.71, 25.80, 21.72, 13.95. HRMS (ESI-TOF) Calcd for C₂₀H₂₃O₄[M−H]⁻: 327.1596; found: 327.1602.

### General Procedure for Hydroxylation of Benzoic Acids

Conditions A: Pd(OAc)₂ (4.5 mg, 0.02 mmol, 2 mol %), L4 (7.3 mg, 0.04 mmol, 4 mol %), carboxylic acid 3 (1.0 mmol), and K₂1PO₄·3H₂O (342.3 mg, 1.5 mmol, 1.5 equiv.) were weighed and placed in an 8 mL vial. Then, CH₃CN (3.0 mL) was added and stirred for 10 min, followed by the addition of H₂O₂(35% aq., 300 μL, 3.5 equiv.). The vial was sealed with a screw cap and stirred at room temperature for 24 h (typically ran at 25° C. unless otherwise noted). Upon completion, the reaction was quenched with saturated solution of Na₂SO₃ (or Na₂S₂O₃) in water until H₂O₂ was completely decomposed. (Tested by the potassium iodide starch test paper). The mixture was diluted with methanol and acidified with formic acid. The solution was filtered through a pad of Celite, and the aqueous layer was extracted with ethyl acetate three times. The combined organic layers were dried over anhydrous Na₂SO₄ and concentrated under vacuum. The crude mixture was purified by flash chromatography (Hexane/EtOAc or DCM/MeOH with 1% AcOH, v/v).

Conditions B: Pd(OAc)₂ (11.0 mg, 0.05 mmol, 5 mol %), L4 (18.2 mg, 0.10 mmol, 10 mol %), carboxylic acid 3 (1.0 mmol), and CsOAc (288.0 mg, 1.5 mmol, 1.5 equiv.) were weighed and placed in a reaction tube. Then, DMA (3.0 mL) was added and stirred for 10 min, followed by the addition of H₂O₂(35% aq., 300 μL, 3.0 equiv.). The vial was sealed with a screw cap and stirred at 60° C. for 24 h. Upon completion, the reaction was quenched with saturated solution of Na₂SO₃ (or Na₂S₂O₃) in water until H₂O₂ was completely decomposed. (Tested by the potassium iodide starch test paper). The mixture was diluted with methanol and acidified with formic acid. The solution was filtered through a pad of Celite, and the aqueous layer was extracted with ethyl acetate three times. The combined organic layers were dried over anhydrous Na₂SO₄ and concentrated under vacuum. The crude mixture was purified by flash chromatography (Hexane/EtOAc or DCM/MeOH with 1% AcOH, v/v).

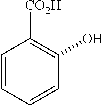

### 2-hydroxybenzoic acid (4a)

Substrate 3a was hydroxylated following general procedure conditions A, the hydroxylated product 4a was obtained as a white solid (130 mg, 94%), ¹H NMR (600 MHz, Methanol-d₄) δ 7.85 (dd, J=7.9, 1.6 Hz, 1H), 7.46 (ddd, J=8.3, 7.2, 1.7 Hz, 1H), 6.94-6.85 (m, 2H). ¹³C NMR (151 MHz, Methanol-d₄) δ 173.51, 163.22, 136.60, 131.53, 120.04, 118.14, 113.88. HRMS (ESI-TOF) Calcd for C₇H₅O₃ [M−H]⁻: 137.0244; found: 137.0240.

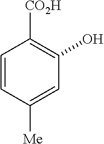

### 2-hydroxy-4-methylbenzoic acid (4b)

Substrate 3b was hydroxylated following general procedure conditions A, the hydroxylated product 4b was obtained as an ivory solid (140 mg, 92%), ¹H NMR (600 MHz, Methanol-d₄) δ 7.72 (d, J=8.0 Hz, 1H), 6.75-6.73 (m, 1H), 6.73-6.70 (m, 1H), 2.33 (s, 3H). ¹³C NMR (151 MHz, Methanol-d₄) δ 173.54, 163.14, 148.11, 131.37, 121.26, 118.22, 111.30, 21.79. HRMS (ESI-TOF) Calcd for C₈H₇O₃ [M−H]⁻: 151.0395; found: 151.0399.

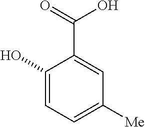

### 2-hydroxy-5-methylbenzoic acid (4c)

Substrate 3c was hydroxylated following general procedure conditions A, the hydroxylated product 4c was obtained as a white solid (130 mg, 85%), ¹H NMR (600 MHz, Methanol-d₄) δ 7.64 (dd, J=2.3, 0.9 Hz, 1H), 7.26 (ddd, J=8.3, 2.3, 0.6 Hz, 1H), 6.80 (d, J=8.4 Hz, 1H), 2.25 (s, 3H). ¹³C NMR (151 MHz, Methanol-d₄) δ 173.50, 161.04, 137.42, 131.19, 129.35, 117.93, 113.42, 20.36 HRMS (ESI-TOF) Calcd for C₈H₇O₃ [M−H]⁻: 151.0395; found: 151.0399.

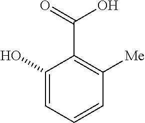

### 2-hydroxy-6-methylbenzoic acid (4d)

Substrate 3d was hydroxylated following general procedure conditions A, the hydroxylated product 4d was obtained as white solid (99.3 mg, 65%), ¹H NMR (600 MHz, Methanol-d₄) δ 7.23 (dd, J=8.3, 7.5 Hz, 1H), 6.76-6.70 (m, 2H), 2.54 (s, 3H). ¹³C NMR (151 MHz, Methanol-d₄) δ 174.86, 163.32, 142.45, 134.43, 123.44, 115.86, 115.07, 23.48. HRMS (ESI-TOF) Calcd for C₈H₇O₃ [M−H]⁻: 151.0395; found: 151.0395.

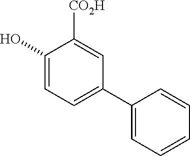

### 4-hydroxy-[1,1′-biphenyl]-3-carboxylic acid (4e)

Substrate 3e was hydroxylated following general procedure conditions A, the hydroxylated product 4e was obtained as a yellow solid (191 mg, 89%), ¹H NMR (600 MHz, Methanol-d₄) δ 8.09 (d, J=2.4 Hz, 1H), 7.74 (dt, J=8.5, 2.2 Hz, 1H), 7.57-7.53 (m, 2H), 7.44-7.39 (m, 2H), 7.34-7.26 (m, 1H), 7.01 (dd, J=8.6, 2.1 Hz, 1H). ¹³C NMR (151 MHz, Methanol-d₄) δ 173.33, 162.64, 141.28, 135.15, 133.59, 129.91, 129.54, 128.02, 127.47, 118.75, 114.21. HRMS (ESI-TOF) Calcd for C₁₃H₉O₃ [M−H]⁻: 213.0552; found: 213.0555.

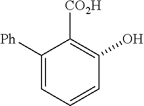

### 3-hydroxy-[1,1′-biphenyl]-2-carboxylic acid (4f)

Substrate 3f was hydroxylated following general procedure conditions A, the hydroxylated product 4f was obtained as a white solid (198 mg, 92%), ¹H NMR (600 MHz, Methanol-d₄) δ 7.35-7.26 (m, 6H), 6.91 (dd, J=8.3, 1.1 Hz, 1H), 6.77 (dd, J=7.5, 1.1 Hz, 1H). ¹³C NMR (151 MHz, Methanol-d₄) δ 173.41, 159.78, 144.81, 143.20, 133.07, 129.37, 128.81, 128.02, 122.80, 117.72, 116.47. HRMS (ESI-TOF) Calcd for C₁₃H₉O₃ [M−H]⁻: 213.0552; found: 213.0553.

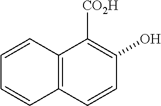

### 2-hydroxy-1-naphthoic acid (4g)

General procedure following conditions A, the hydroxylated product 4g was obtained as a white solid (172 mg, 91%), ¹H NMR (600 MHz, Methanol-d₄) δ 8.86 (dd, J=8.8, 1.0 Hz, 1H), 7.92 (d, J=9.0 Hz, 1H), 7.75 (dd, J=8.1, 1.5 Hz, 1H), 7.51 (dd, J=8.6, 6.9, 1.5 Hz, 1H), 7.33 (ddd, J=8.0, 6.8, 1.1 Hz, 1H), 7.11 (d, J=9.0 Hz, 1H). ¹³C NMR (151 MHz, Methanol-d₄) δ 175.34, 165.31, 137.51, 133.63, 130.04, 129.93, 129.19, 126.49, 124.50, 119.95, 106.10. HRMS (ESI-TOF) Calcd for C₁₁H₇O₃ [M−H]⁻: 187.0395; found: 187.0398.

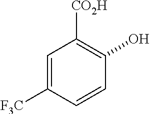

### 2-hydroxy-5-(trifluoromethyl)benzoic acid (4h)

Substrate 3h was hydroxylated following general procedure conditions A but under 60° C., the hydroxylated product 4h was obtained as an ivory solid (167 mg, 81%), ¹H NMR (600 MHz, Methanol-d₄) δ 8.12 (dd, J=2.4, 1.0 Hz, 1H), 7.73 (ddd, J=8.8, 2.4, 0.7 Hz, 1H), 7.09 (dd, J=8.8, 0.8 Hz, 1H). ¹³C NMR (151 MHz, Methanol-d₄) δ 172.42, 165.75, 132.96 (q, J=3.5 Hz), 128.96 (q, J=4.2 Hz), 125.45 (q, J=270.2 Hz), 122.32 (q, J=33.1 Hz), 119.30, 114.17. ¹⁹F NMR (376 MHz, Methanol-d₄) δ −65.77. HRMS (ESI-TOF) Calcd for C₈H₄F₃O₃[M−H]⁻: 205.0113; found: 205.0114.

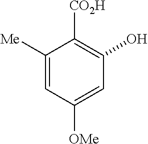

### 2-hydroxy-4-methoxy-6-methylbenzoic acid (4i)

Substrate 3i was hydroxylated following general procedure conditions A, the hydroxylated product 4i was obtained as a yellow solid (127 mg, 70%), ¹H NMR (600 MHz, Methanol-d₄) δ 6.34-6.30 (m, 2H), 3.80 (s, 3H), 2.54 (s, 3H). ¹³C NMR (151 MHz, Methanol-d₄) δ 174.96, 166.99, 165.37, 144.90, 111.46, 106.46, 99.61, 55.74, 24.29. HRMS (ESI-TOF) Calcd for C₉H₉O₄ [M−H]⁻: 181.0501; found: 181.0505.

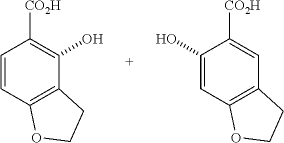

### 4-hydroxy-2,3-dihydrobenzofuran-5-carboxylic (4j, left)

### 6-hydroxy-2,3-dihydrobenzofuran-5-carboxylic acid (4j′, right)

Substrate 3j was hydroxylated following general procedure conditions A, the hydroxylated products 4j and 4j′ were obtained as inseparable mixture (1:1), (140 mg, 78%), ¹H NMR (600 MHz, Methanol-d₄) δ 7.70-7.66 (m, 1H), 7.66-7.63 (m, 1H), 6.33-6.28 (m, 1H), 6.24 (d, J=1.9 Hz, 1H), 4.66-4.62 (m, 2H), 4.61-4.57 (m, 2H), 3.16-3.08 (m, 4H). ¹³C NMR (151 MHz, Methanol-d₄) δ 173.46, 168.10, 167.78, 165.48, 160.69, 146.79, 133.11, 127.41, 120.30, 113.99, 107.68, 102.62, 98.21, 73.87, 73.81, 29.04, 27.09. (Peaks for —COOH are overlapped.) HRMS (ESI-TOF) Calcd for C₉H₇O₄ [M−H]⁻: 179.0344; found: 179.0346.

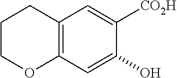

### 7-hydroxychromane-6-carboxylic acid (4k)

Substrate 3k was hydroxylated following general procedure conditions A, the hydroxylated product 4k was obtained as a brown solid (183 mg, 94%), ¹H NMR (600 MHz, Methanol-d₄) δ 7.55 (t, J=1.1 Hz, 1H), 6.22 (s, 1H), 4.34-3.98 (m, 2H), 2.85-2.49 (m, 2H), 2.11-1.82 (m, 2H). ¹³C NMR (151 MHz, Methanol-d₄) δ 173.34, 162.89, 162.37, 132.93, 115.48, 106.85, 104.33, 68.07, 25.15, 23.40. HRMS (ESI-TOF) Calcd for C₁₀H₉O₄ [M−H]⁻: 193.0501; found: 193.0500.

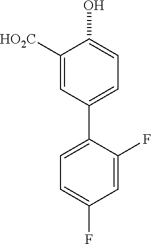

### 2′,4′-difluoro-4-hydroxy-[1,1′-biphenyl]-3-carboxylic acid (41)

Substrate 31 was hydroxylated following general procedure conditions A but under 60° C., the hydroxylated product 41 was obtained as a yellow solid (241 mg, 96%), ¹H NMR (600 MHz, Methanol-d₄) δ 7.99 (s, 1H), 7.62 (d, J=8.6 Hz, 1H), 7.47 (td, J=8.3, 5.9 Hz, 1H), 7.07-6.98 (m, 3H). ¹³C NMR (151 MHz, Methanol-d₄) δ 172.76, 164.44 (dd, J=247.6, 12.0 Hz), 162.98, 161.06 (dd, J=248.7, 12.1 Hz), 137.01, 132.55 (d, J=9.3 Hz), 132.51 (d, J=9.6 Hz), 131.86, 127.14, 125.63 (dd, J=13.6, 3.9 Hz), 118.57, 112.71 (dd, J=21.4, 3.8 Hz), 105.13 (dd, J=27.0, 25.8 Hz). ¹⁹F NMR (376 MHz, Methanol-d₄) δ −113.68 (d, J=6.8 Hz), −115.69 (d, J=7.3 Hz). HRMS (ESI-TOF) Calcd for C₁₃H₇F₂O₃[M−H]⁻: 249.0363; found: 249.0365.

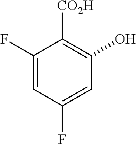

### 2,4-difluoro-6-hydroxybenzoic acid (4m)

Substrate 3m was hydroxylated following general procedure conditions B, the hydroxylated product 4m was obtained as a white solid (126 mg, 72%), ¹H NMR (600 MHz, Methanol-d₄) δ 6.52-6.45 (m, 2H). ¹³C NMR (151 MHz, Methanol-d₄) δ 171.60 (d, J=3.6 Hz), 167.79 (dd, J=251.0, 15.5 Hz), 166.21 (dd, J=16.5, 6.1 Hz), 165.19 (dd, J=261.6, 17.2 Hz), 101.20 (dd, J=13.5, 3.5 Hz), 101.03 (dd, J=24.4, 4.4 Hz), 97.24-96.59 (m). ¹⁹F NMR NMR (376 MHz, Methanol-d₄) δ −102.64 (d, J=12.7 Hz), −102.82 (d, J=12.7 Hz). HRMS (ESI-TOF) Calcd for C₇H₃F₂O₃[M−H]⁻: 173.0050; found: 173.0054.

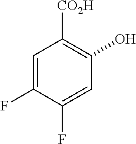

### 4,5-difluoro-2-hydroxybenzoic acid (4n)

Substrate 3n was hydroxylated following general procedure conditions B, the hydroxylated product 4n was obtained as an ivory solid (128 mg, 73%), ¹H NMR (600 MHz, Methanol-d₄) δ 7.72-7.65 (m, 1H), 6.87-6.79 (m, 1H). ¹³C NMR (151 MHz, Methanol-d₄) δ 171.97, 160.70 (dd, J=11.8, 1.7 Hz), 156.11 (dd, J=254.6, 14.3 Hz), 144.63 (dd, J=240.2, 13.4 Hz), 118.84 (dd, J=19.5, 2.6 Hz), 110.02, 106.78 (d, J=20.3 Hz). ¹⁹F NMR (376 MHz, Methanol-d₄) δ −128.80 (d, J=21.8 Hz), −151.10 (d, J=21.7 Hz). HRMS (ESI-TOF) Calcd for C₇H₃F₂O₃[M−H]⁻: 173.0050; found: 173.0054.

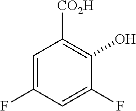

### 3,5-difluoro-2-hydroxybenzoic acid (4o)

Substrate 3o was hydroxylated following general procedure conditions B, the hydroxylated product 4o was obtained as a pink solid (113 mg, 65%), ¹H NMR (600 MHz, Methanol-d₄) δ 7.38 (ddd, J=8.7, 3.1, 2.0 Hz, 1H), 7.25 (ddd, J=11.3, 8.3, 3.1 Hz, 1H). ¹³C NMR (151 MHz, Methanol-d₄) δ 171.83, 154.95 (dd, J=238.8, 10.5 Hz), 152.53 (d, J=247.5 Hz), 148.38 (d, J=12.5 Hz), 116.85, 111.73 (d, J=23.8 Hz), 110.88 (dd, J=27.6, 21.9 Hz). ¹⁹F NMR (376 MHz, Methanol-d₄) δ −124.23, −134.42. HRMS (ESI-TOF) Calcd for C₇H₃F₂O₃[M−H]⁻: 173.0050; found: 173.0048.

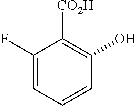

### 2-fluoro-6-hydroxybenzoic acid (4p)

Substrate 3p was hydroxylated following general procedure conditions B, the hydroxylated product 4p was obtained as a yellow solid (96.2 mg, 62%), ¹H NMR (600 MHz, Methanol-d₄) δ 7.40 (td, J=8.5, 6.0 Hz, 1H), 6.74 (dt, J=8.5, 1.0 Hz, 1H), 6.62 (ddd, J=10.9, 8.2, 1.0 Hz, 1H). ¹³C NMR (151 MHz, Methanol-d₄) δ 171.72, 164.40, 163.93 (d, J=258.8 Hz), 136.16, 136.08, 113.96, 107.58 (d, J=23.5 Hz). ¹⁹F NMR (376 MHz, Methanol-d₄) δ −107.63. HRMS (ESI-TOF) Calcd for C₇H₄FO₃ [M−H]⁻: 155.0144; found: 155.0147.

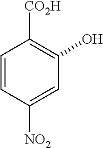

### 2-hydroxy-4-nitrobenzoic acid (4q)

Substrate 3q was hydroxylated following general procedure conditions B, the hydroxylated product 4q was obtained as a yellow solid, (136 mg, 74%), ¹H NMR (600 MHz, Methanol-d₄) δ 8.09 (dd, J=8.6, 0.5 Hz, 1H), 7.74-7.69 (m, 2H). ¹³C NMR (151 MHz, Methanol-d₄) δ 172.06, 163.45, 153.41, 133.04, 119.19, 114.29, 113.19. HRMS (ESI-TOF) Calcd for C₇H₄NO₅ [M−H]⁻: 182.0089; found: 182.0090.

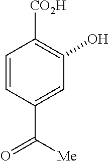

### 4-acetyl-2-hydroxybenzoic acid (4r)

Substrate 3r was hydroxylated following general procedure conditions B, the hydroxylated product 4r was obtained as an ivory solid (125 mg, 69%), ¹H NMR (600 MHz, Methanol-d₄) δ 7.97 (d, J=8.1 Hz, 1H), 7.48-7.44 (m, 2H), 2.60 (s, 3H). ¹³C NMR (151 MHz, Methanol-d₄) δ 199.90, 163.00, 143.18, 132.16, 120.12, 119.04, 117.68, 26.95. (One peak corresponds —COOH was not detected) HRMS (ESI-TOF) Calcd for C₉H₇O₄ [M−H]⁻: 179.0344; found: 179.0344.

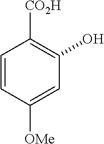

### 2-hydroxy-4-methoxybenzoic acid (4s)

Substrate 3s was hydroxylated following general procedure conditions B, the hydroxylated product 4s was obtained as an ivory solid (102 mg, 61%), ¹H NMR (600 MHz, Methanol-d₄) δ 7.76 (d, J=8.7 Hz, 1H), 6.49-6.40 (m, 2H), 3.82 (s, 3H). ¹³C NMR (151 MHz, Methanol-d₄) δ 173.38, 167.14, 165.37, 132.89, 107.93, 106.78, 101.58, 55.97. HRMS (ESI-TOF) Calcd for C₈H₇O₄ [M−H]⁻: 167.0344; found: 167.0346.

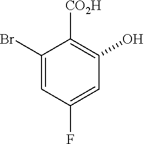

### 2-bromo-4-fluoro-6-hydroxybenzoic acid (4t)

Substrate 3t was hydroxylated following general procedure conditions B but K₂HPO₄·3H₂O was used instead of CsOAc and the reaction was run for 48 h, the hydroxylated product 4t was obtained as an ivory solid (157 mg, 67%), ¹H NMR (600 MHz, Methanol-d₄) δ 6.85 (dd, J=8.6, 2.6 Hz, 1H), 6.55 (dd, J=10.2, 2.6 Hz, 1H). ¹³C NMR (151 MHz, Methanol-d₄) δ 173.96, 164.91 (d, J=249.8 Hz), 164.81 (d, J=14.0 Hz), 124.95 (d, J=12.9 Hz), 118.13, 113.10 (d, J=25.3 Hz), 103.64 (d, J=23.2 Hz). ¹⁹F NMR (376 MHz, Methanol-d₄) δ −111.12. HRMS (ESI-TOF) Calcd for C₇H₃BrFO₃ [M−H]⁻: 232.9250; found: 232.9251.

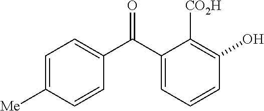

### 2-hydroxy-6-(4-methylbenzoyl)benzoic acid (4u)

Substrate 3u was hydroxylated following general procedure conditions B to afford the hydroxylated product 4u (244 mg, 95%), ¹H NMR (600 MHz, Methanol-d₄) δ 7.60 (d, J=8.0 Hz, 2H), 7.53 (dd, J=8.4, 7.4 Hz, 1H), 7.26 (d, J=8.0 Hz, 2H), 7.08 (dd, J=8.4, 1.1 Hz, 1H), 6.76 (dd, J=7.4, 1.1 Hz, 1H), 2.39 (s, 3H). ¹³C NMR (151 MHz, Methanol-d₄) δ200.93, 162.72, 144.93, 144.57, 136.90, 133.57, 130.38, 130.06, 129.85, 118.45, 117.84, 21.57. (One peak corresponds —COOH was not detected) HRMS (ESI-TOF) Calcd for C₁₅H₁₁O₄[M−H]⁻: 255.0657; found: 255.0657.

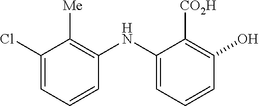

### 2-((3-chloro-2-methylphenyl)amino)-6-hydroxybenzoic acid (4v)

Substrate 3v was hydroxylated following general procedure conditions B, the hydroxylated product 4v was obtained as a brown solid (255 mg, 92%), ¹H NMR (400 MHz, Methanol-d₄) δ 7.26-7.19 (m, 1H), 7.11-7.03 (m, 2H), 6.97 (t, J=8.2 Hz, 1H), 6.26-6.04 (m, 2H), 2.28 (s, 3H). ¹³C NMR (100 MHz, Methanol-d₄) δ 164.47, 150.04, 143.43, 136.18, 133.59, 131.48, 127.84, 125.18, 123.01, 106.38, 104.66, 104.18, 15.23. (One peak corresponds —COOH was not detected) HRMS (ESI-TOF) Calcd for C₁₄H₁₁ClNO₃ [M−H]⁻: 276.0427; found: 276.0427.

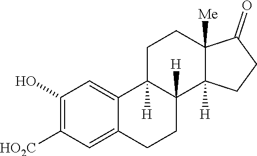

### (8R,9S,13S,14S)-2-hydroxy-13-methyl-17-oxo-7,8,9,11,12,13,14,15,16,17-decahydro-6H-cyclopenta[a]phenanthrene-3-carboxylic acid (4w)

Substrate 3w (0.5 mmol) was hydroxylated following general procedure conditions B but under room temperature, K₂HPO₄·3H₂O was used instead of CsOAc. the hydroxylated product 4w was obtained as an ivory solid (96.2 mg, 61%), ¹H NMR (600 MHz, Methanol-d₄) δ 7.55 (s, 1H), 6.85 (s, 1H), 2.88-2.78 (m, 2H), 2.53-2.47 (m, 1H), 2.40-2.35 (m, 1H), 2.31-2.26 (m, 1H), 2.18-2.01 (m, 3H), 1.93-1.87 (m, 1H), 1.72-1.39 (m, 6H), 0.93 (s, 3H). ¹³C NMR (151 MHz, Methanol-d₄) δ 223.44, 173.40, 160.97, 149.76, 131.28, 128.61, 114.55, 111.70, 51.79, 49.17, 46.03, 39.11, 36.69, 32.71, 29.31, 27.61, 26.58, 22.51, 14.21. HRMS (ESI-TOF) Calcd for C₁₉H₂₁O₄[M−H]⁻: 313.1440; found: 313.1439.

### 3. Synthetic Application

### 3.1. Large Scale Reactions

Attention: Peroxides are particularly dangerous when they are concentrated. Large scale reactions should be quenched and tested by the potassium iodide starch test paper.

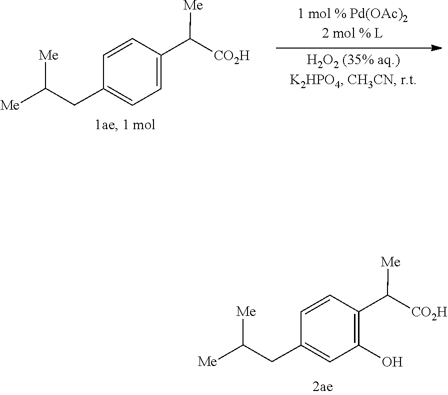 [SMILES: CC(C)Cc1ccc(C(C)C(=O)O)cc1 ; CC(C)Cc1ccc(C(C)C(=O)O)c(O)c1]

Pd(OAc)₂ (2.24 g, 0.01 mol, 1 mol %), L4 (3.62 g, 0.02 mol, 2 mol %), carboxylic acid 1ae (206 g, 1.0 mol), and K₂HPO₄ (260 g, 1.5 mol, 1.5 equiv.) were weighed and placed in a 3 L round bottom glass flask. Then, CH₃CN (2.0 L) was added and stirred vigorously for 30 min and H₂O₂(35% aq., 300 mL, 3.5 equiv.) was carefully added in several portions. The mixture was stirred at ambient temperature for 24 h. After that, the reaction was monitored by LCMS and H₂O₂(35% aq., 100 mL, 1.1 equiv.) was added and stirred vigorously for an additional 24 h. Upon completion, the reaction was carefully quenched with Na₂S₂O₃ aqueous solution until H₂O₂ was completely decomposed. The mixture was acidified with 1 M aqueous HCl and filtered through Celite then washed with EtOAc. The aqueous layer was extracted with ethyl acetate three times. The combined organic layers were washed with brine then dried over anhydrous Na₂SO₄ and concentrated under vacuum. The crude mixture was purified by multiple recrystallizations with Hexane/DCM. The mother liquor was further purified by flash chromatography (Hexane/EtOAc with 1% AcOH, v/v). The total hydroxylated product 2ae was obtained as a white solid (179.2 g, 81%).

### Fig. S1. C(sp²)-H hydroxylation of Ibuprofen

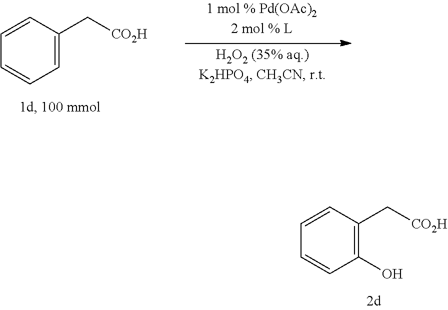 [SMILES: O=C(O)Cc1ccccc1 ; O=C(O)Cc1ccccc1O]

Pd(OAc)₂ (224 mg, 1 mmol, 1 mol %), L4 (362 mg, 2 mmol, 2 mol %), carboxylic acid 1d (13.6 g, 100 mmol), and K₂HPO₄ (26.0 g, 150 mmol, 1.5 equiv.) were weighed and placed in a 500 mL round bottom glass flask. Then, CH₃CN (250 mL) was added and stirred vigorously for 30 min and H₂O₂(35% aq., 30 mL, 3.5 equiv.) was carefully added in several portions. The mixture was stirred at ambient temperature for 48 h. Upon completion, the reaction was carefully quenched with Na₂S₂O₃ aqueous solution until H₂O₂ was completely decomposed. The mixture was acidified with 1 M aqueous HCl and filtered through Celite then washed with EtOAc. The aqueous layer was extracted with ethyl acetate three times. The combined organic layers were dried over anhydrous Na₂SO₄ and concentrated under vacuum. The crude mixture was purified by multiple recrystallizations with Hexane/EtOAc. The mother liquor was further purified by flash chromatography (Hexane/EtOAc with 1% AcOH, v/v). The hydroxylated product 2d was obtained as a white solid (12.4 g, 82%).

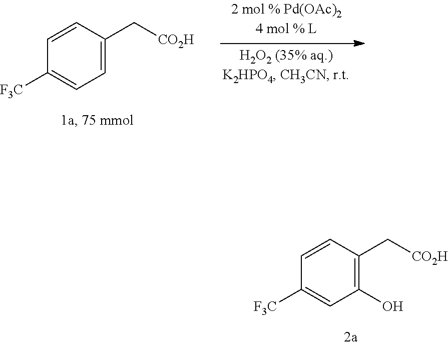 [SMILES: O=C(O)Cc1ccc(C(F)(F)F)cc1 ; O=C(O)Cc1ccc(C(F)(F)F)cc1O]

Pd(OAc)₂ (336 mg, 1.5 mmol, 2 mol %), L4 (543 mg, 3 mmol, 2 mol %), carboxylic acid 1d (15.3 g, 75 mmol), and K₂HPO₄ (19.7 g, 113 mmol, 1.5 equiv.) were weighed and placed in a 500 mL round bottom glass flask. Then, CH₃CN (200 mL) was added and stirred vigorously for 30 min and H₂O₂(35% aq., 22.5 mL, 3.5 equiv.) was carefully added in several portions. The mixture was stirred at ambient temperature for 48 h. Upon completion, the reaction was carefully quenched with Na₂S₂O₃ aqueous solution until H₂O₂ was completely decomposed. The mixture was acidified with 1 M aqueous HCl and filtered through Celite then washed with EtOAc. The aqueous layer was extracted with ethyl acetate three times. The combined organic layers were dried over anhydrous Na₂SO₄ and concentrated under vacuum. The crude mixture was purified by flash chromatography (Hexane/EtOAc with 1% AcOH, v/v). The hydroxylated product 2d was obtained as a yellow solid (12.6 g, 76%).

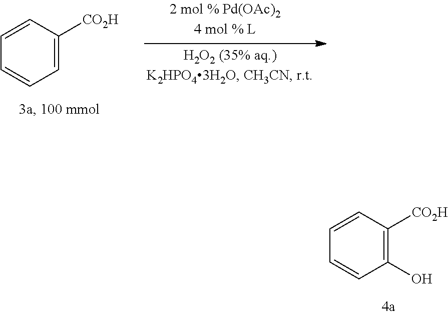 [SMILES: O=C(O)c1ccccc1 ; O=C(O)c1ccccc1O]

Pd(OAc)₂ (448 mg, 2 mmol, 2 mol %), L4 (724 mg, 4 mmol, 4 mol %), carboxylic acid 1d (12.2 g, 100 mmol), and K₂HPO₄ (26.0 g, 150 mmol, 1.5 equiv.) were weighed and placed in a 500 mL round bottom glass flask. Then, CH₃CN (250 mL) was added and stirred vigorously for 30 min and H₂O₂(35% aq., 30 mL, 3.5 equiv.) was carefully added in several portions. The mixture was stirred at ambient temperature for 48 h. Upon completion, the reaction was carefully quenched with Na₂S₂O₃ aqueous solution until H₂O₂ was completely decomposed. The mixture was acidified with 1 M aqueous HCl and filtered through Celite then washed with EtOAc. The aqueous layer was extracted with ethyl acetate three times. The combined organic layers were dried over anhydrous Na₂SO₄ and concentrated under vacuum. The crude mixture was purified by multiple recrystallizations. The hydroxylated product 2d was obtained as a white solid (10.6 g, 77%).

### 3.2. Further Derivatizations of Phenols Products

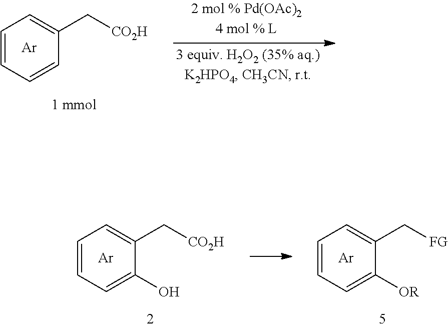

Pd(OAc)₂ (4.5 mg, 0.02 mmol, 2 mol %), L4 (7.3 mg, 0.04 mmol, 4 mol %), carboxylic acid 1d or 1a (1.0 mmol), and K₂HPO₄ (260.0 mg, 1.5 mmol, 1.5 equiv.) were weighed and placed in an 8 mL vial. Then, CH₃CN (3.0 mL) was added and stirred for 5 min, followed by the addition of H₂O₂(35% aq., 300 μL, 3.5 equiv.). The vial was sealed with a screw cap and stirred at ambient temperature for 24 h. Upon completion, the reaction was quenched with aqueous Na₂SO₃ solution and acidified with 1 M aqueous HCl. The solution was filtered through a pad of Celite then washed with ethyl acetate. The aqueous layer was extracted with ethyl acetate three times and the combined organic layers were dried over anhydrous Na₂SO₄ and concentrated under vacuum. The crude mixture 2d or 2a was used in the next step without further purification.

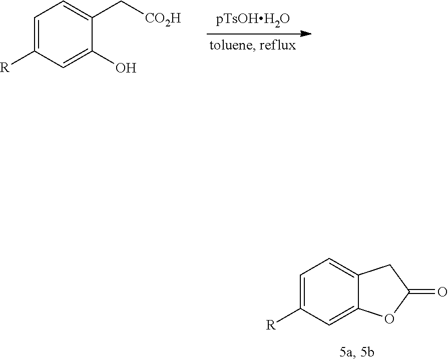

To a 25 mL flask provided with Dean-Stark tramp and magnetic stirrer was added crude 2d or 2a in 15 mL of toluene and catalytic amounts of p-TsOH (0.1 mmol). The mixture was refluxed for overnight with removal of water and then the residual solvent was removed at reduced pressure. The crude mixture was purified by flash column chromatography (Hexane/EtOAc) to give the corresponding lactone products.

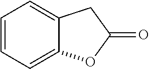

### benzofuran-2(3H)-one (5a)

[CAS: 553-86-6] Yellow oil, 91.1 mg, 68%, ¹H NMR (400 MHz, Chloroform-d) δ 7.34-7.27 (m, 2H), 7.17-7.09 (m, 2H), 3.74 (s, 2H). ¹³C NMR (100 MHz, Chloroform-d) δ 174.29, 154.87, 129.07, 124.81, 124.27, 123.21, 110.97, 33.17. The NMR data matches the reported data⁵.

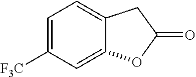

### 6-(trifluoromethyl)benzofuran-2(3H)-one (5b)

Yellow solid, 123.2 mg, 61%, ¹H NMR (400 MHz, Chloroform-d) δ 7.46-7.39 (m, 2H), 7.38-7.34 (m, 1H), 3.81 (s, 2H). ¹³C NMR (151 MHz, Chloroform-d) δ 172.85, 154.84, 131.78 (q, J=33.1 Hz), 127.17, 123.60 (q, J=272.4 Hz), 125.23, 121.31 (q, J=4.0 Hz), 108.23 (q, J=3.9 Hz), 32.94. ¹⁹F NMR (376 MHz, CDCl₃) δ −65.22. HRMS (ESI-TOF) Calcd for C₉H₄F₃O₂[M−H]⁻: 201.0169; found: 201.0168.

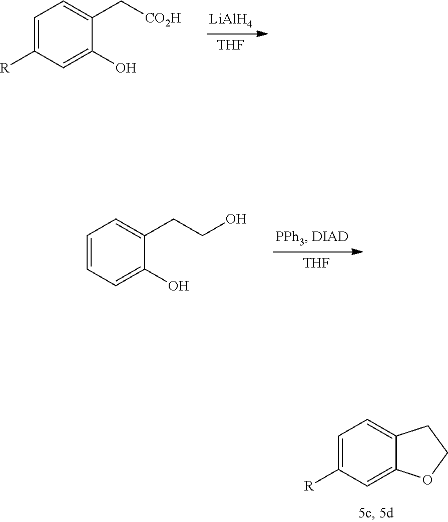

To a solution of crude 2d or 2a in THE (2.0 mL) was added lithium aluminum hydride solution (2.0 M in THF, 1.0 mL, 2 mmol) at 0° C. The reaction mixture was warmed to ambient temperature and stirred for 6 h. Upon completion, the mixture was diluted with 10 mL Et₂O and carefully quenched with 1 M aqueous HCl at 0° C. until the solution becomes clear. The aqueous layer was extracted with EtOAc. The combined organic layers were washed with brine then dried and evaporated under reduced pressure. The crude product was used in the next step without further purification.

To a solution of the crude alcohol in THE (2.0 mL) was added PPh₃ (314.4 mg, 1.2 mmol) and diisopropyl azodicarboxylate (303.3 mg, 1.5 mmol). The mixture was stirred at ambient temperature until the diol was no longer apparent by TLC. Upon completion, the reaction was diluted with 20 mL diethyl ether and stirred for 30 min. The mixture was filtered through Celite and evaporated under reduced pressure. The crude product was purified by flash chromatography (Hexane/EtOAc=10:1, v/v) to give the corresponding product.

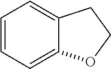

### 2,3-dihydrobenzofuran (5c)

[CAS: 496-16-2] Colourless liquid, 87.6 mg, 73%, ¹H NMR (600 MHz, Chloroform-d) δ 7.20 (d, J=7.2 Hz, 1H), 7.11 (t, J=7.7 Hz, 1H), 6.84 (td, J=7.4, 1.0 Hz, 1H), 6.79 (d, J=7.7 Hz, 1H), 4.56 (t, J=8.7 Hz, 2H), 3.21 (t, J=8.6 Hz, 2H). ¹³C NMR (151 MHz, Chloroform-d) δ 160.14, 128.06, 127.00, 125.04, 120.46, 109.49, 71.14, 29.88. The NMR data matches the reported data⁶.

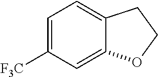

### 6-(trifluoromethyl)-2,3-dihydrobenzofuran (5d)

[CAS: 1391072-82-4] yellow liquid, 120.8 mg, 64%, ¹H NMR (600 MHz, Chloroform-d) δ 7.26 (d, J=8.1 Hz, 1H), 7.11 (d, J=7.7 Hz, 1H), 7.00 (s, 1H), 4.63 (t, J=8.8 Hz, 2H), 3.25 (tt, J=8.7, 1.3 Hz, 2H). ¹³C NMR (151 MHz, Chloroform-d) δ 160.47, 131.33, 131.71 (q, J=32.2 Hz), 125.16, 124.28 (q, J=272.1 Hz), 117.65 (q, J=4.2 Hz), 106.47 (q, J=3.8 Hz), 71.80, 29.65. ¹⁹F NMR (376 MHz, Chloroform-d) 6-64.91. The NMR data matches the reported data⁷.

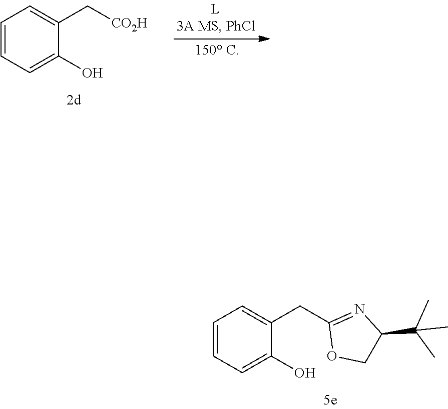 [SMILES: O=C(O)Cc1ccccc1O ; CC(C)(C)[C@H]1COC(Cc2ccccc2O)=N1]

To a solution of crude 2d in PhCl (4.0 mL) was added L-tert-Leucinol (156 uL, 1.2 mmol) and activated 3A molecule sieve (200 mg). The reaction mixture was heated to 150° C. and stirred for 24 h. After cooling down to room temperature, the reaction mixture was diluted with EtOAc, filtered, and evaporated under reduced pressure. The crude product was purified by flash chromatography (hexane/EtOAc=4:1, v/v) to give product 5e as a white solid (177.1 mg, 76%).

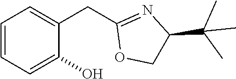

### (S)-2-((4-(tert-butyl)-4,5-dihydrooxazol-2-yl)methyl)phenol (5e)

[CAS: 1816267-52-3]¹H NMR (600 MHz, Chloroform-d) δ 10.60 (br s, 1H), 7.19 (td, J=7.7, 1.7 Hz, 1H), 7.05 (dd, J=7.7, 1.7 Hz, 1H), 6.98 (dd, J=8.1, 1.4 Hz, 1H), 6.83 (td, J=7.4, 1.3 Hz, 1H), 4.25 (dd, J=10.2, 8.8 Hz, 1H), 4.13 (t, J=8.4 Hz, 1H), 3.89 (dd, J=10.1, 8.0 Hz, 1H), 3.69-3.57 (m, 2H), 0.87 (s, 9H). ¹³C NMR (151 MHz, Chloroform-d) δ 168.94, 156.60, 130.64, 129.16, 122.11, 120.25, 119.07, 74.93, 69.73, 33.65, 31.69, 25.72. The NMR data matches the reported data⁸.

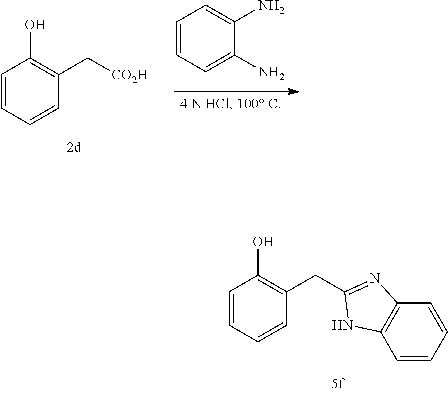 [SMILES: O=C(O)Cc1ccccc1O ; Nc1ccccc1N ; Oc1ccccc1Cc1nc2ccccc2[nH]1]

To a solution of crude 2d in 4M aqueous HCl (2.0 mL) was added o-Phenylenediamine (129 mg, 1.2 mmol). The reaction mixture was heated to 100° C. and stirred for 24 h. After cooling down to room temperature, a saturated aqueous NaHCO₃ solution (20 mL) was added and extracted with EtOAc three times. The combined organic layers were washed with brine then dried and evaporated under reduced pressure. The crude product was purified by flash chromatography (DCM-MeOH 20:1, v/v) to give product 5f as a white solid (105.3 mg, 47%).

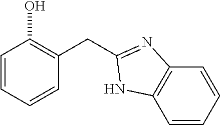

### 2-((1H-benzo[d]imidazol-2-yl)methyl)phenol (5f)

[CAS: 3416-07-7]¹H NMR (600 MHz, Methanol-d₄) δ 7.50-7.44 (m, 2H), 7.18-7.13 (m, 2H), 7.12-7.05 (m, 2H), 6.83 (dd, J=8.0, 1.2 Hz, 1H), 6.77 (td, J=7.5, 1.2 Hz, 1H), 4.20 (s, 2H). ¹³C NMR (151 MHz, Methanol-d₄) δ 155.13, 154.26, 138.15, 129.97, 127.95, 123.21, 121.71, 119.36, 114.82, 114.00, 29.40. The NMR data matches the reported data⁹.

### 3.3. Synthesis of F3—Coumestan and F3-Ptercarpene Derivatives

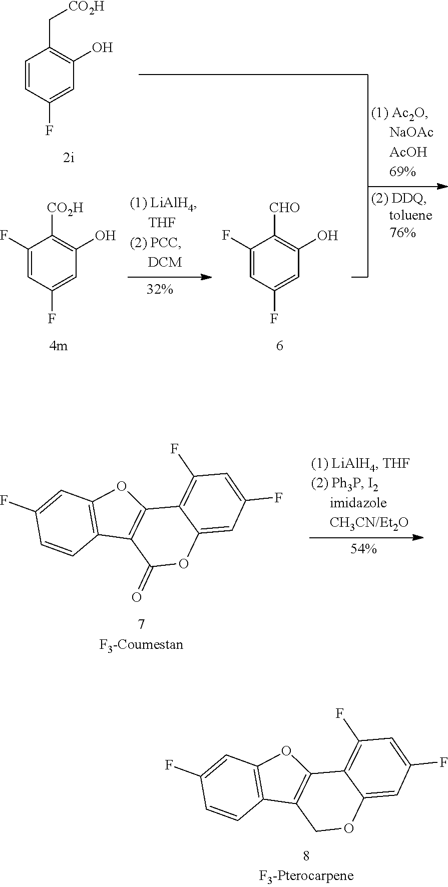 [SMILES: O=C(O)Cc1ccc(F)cc1O ; O=C(O)c1c(O)cc(F)cc1F ; O=Cc1c(O)cc(F)cc1F ; O=c1oc2cc(F)cc(F)c2c2oc3cc(F)ccc3c12 ; Fc1cc(F)c2c(c1)OCc1c-2oc2cc(F)ccc12]

### Synthesis of Compound 6

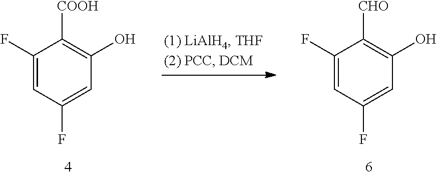 [SMILES: O=C(O)c1c(O)cc(F)cc1F ; O=Cc1c(O)cc(F)cc1F]

To a solution of 4 (1.044 g, 6 mmol) in THE (10.0 mL) was added lithium aluminum hydride solution (2.0 M in THF, 4.5 mL, 9 mmol) at 0° C. The reaction mixture was heated to 50° C. and stirred for 6 h. After cooling down to room temperature, the mixture was diluted with 20 mL of Et₂O and carefully quenched with 1 M aqueous HCl at 0° C. until the solution becomes clear. The aqueous layer was extracted with Et₂O. The combined organic layers were washed with brine then dried and evaporated under reduced pressure. The crude product was used in the next step without further purification.

To a solution of crude alcohol in DCM (20 mL) was added Pyridinium chlorochromate (1.293 mg, 6 mmol, 1.0 equiv.) and Celite (1 g). The mixture was stirred at room temperature for 2 h. Upon completion, the mixture was filtered and directly purified by flash chromatography (Hexane/EtOAc=10:1 as eluent) to give compound 6 as a colorless oil (This compound is extremely volatile).

### Synthesis of Compound 7

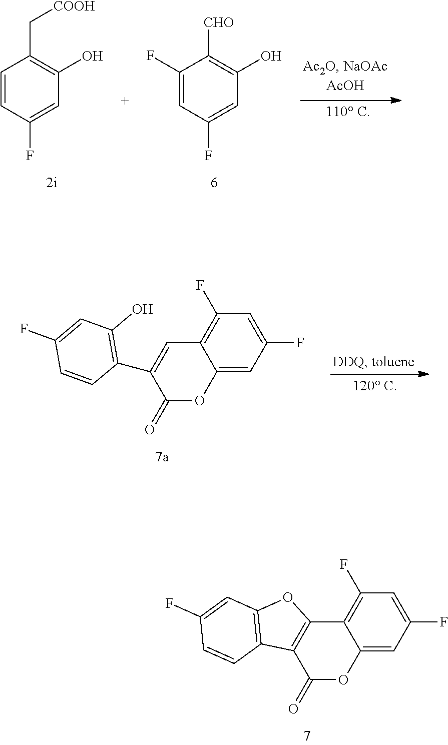 [SMILES: O=C(O)Cc1ccc(F)cc1O ; O=Cc1c(O)cc(F)cc1F ; O=c1oc2cc(F)cc(F)c2cc1-c1ccc(F)cc1O ; O=c1oc2cc(F)cc(F)c2c2oc3cc(F)ccc3c12]

To a solution of 2i (170 mg, 1 mmol) and 6 (190 mg, 1.2 mmol) in AcOH (4.0 mL) was added NaOAc (410 mg, 5 mmol) and Ac₂O (227 uL, 2.4 mmol). The reaction mixture was heated to 110° C. and stirred for 20 h. After cooling down to room temperature, a saturated aqueous NaHCO₃ solution (20 mL) was added and extracted with EtOAc three times. The combined organic layers were washed with brine then dried and evaporated under reduced pressure. The crude product was purified by flash chromatography (Hexane/EtOAc=5:1, v/v) to give product 7a as a white solid (201.5 mg, 69%).

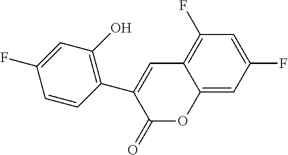 [SMILES: O=c1oc2cc(F)cc(F)c2cc1-c1ccc(F)cc1O]

### 5,7-difluoro-3-(4-fluoro-2-hydroxyphenyl)-2H-chromen-2-one (7a)

¹H NMR (600 MHz, Acetone-d₆) δ 9.15 (s, 1H), 8.05 (s, 1H), 7.44 (dd, J=8.5, 6.7 Hz, 1H), 7.15-7.06 (m, 2H), 6.78-6.68 (m, 2H). ¹³C NMR (151 MHz, Acetone-d₆) δ 164.85 (dd, J=251.6, 14.8 Hz), 164.52 (d, J=245.7 Hz), 159.90 (dd, J=254.9, 15.4 Hz), 159.54, 157.54 (d, J=11.6 Hz), 155.98 (dd, J=15.6, 7.6 Hz), 134.76, 133.34 (d, J=15.1 Hz), 125.49, 119.36 (d, J=3.8 Hz), 107.39 (dd, J=19.3, 3.3 Hz), 107.06 (d, J=21.7 Hz), 104.01 (d, J=24.3 Hz), 101.06 (dd, J=26.0, 4.4 Hz), 100.72 (dd, J=27.3, 24.3 Hz). ¹⁹F NMR (376 MHz, Acetone-d₆) δ −105.29 (d, J=7.8 Hz), −112.73, −117.22 (d, J=7.8 Hz). HRMS (ESI-TOF) Calcd for C₁₅H₈F₃O₃[M+H]⁺: 293.0426; found: 293.0429.

To a solution of 7a (0.69 mmol) in toluene (8.0 mL) was added DDQ (317.8 mg, 1.4 mmol). The reaction mixture was heated to 120° C. and stirred for 12 h. After cooling down to room temperature, the mixture was filtered through Celite and evaporated under reduced pressure. The crude product was purified by flash chromatography (Hexane/EtOAc=10:1, v/v) to give product 7 as a white solid (152.1 mg, 76%).

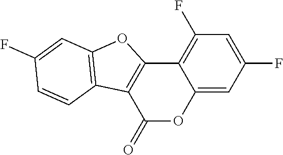 [SMILES: O=c1oc2cc(F)cc(F)c2c2oc3cc(F)ccc3c12]

### 1,3,9-trifluoro-6H-benzofuro[3,2-c]chromen-6-one (7)

¹H NMR (600 MHz, Chloroform-d) δ 8.06 (dd, J=8.6, 5.3 Hz, 1H), 7.44 (dd, J=8.3, 2.2 Hz, 1H), 7.28-7.22 (m, 1H), 7.08 (dt, J=8.9, 2.0 Hz, 1H), 6.94 (ddd, J=9.9, 8.8, 2.3 Hz, 1H). ¹³C NMR (151 MHz, Chloroform-d) δ 164.25 (dd, J=255.3, 13.7 Hz), 162.23 (d, J=247.8 Hz), 157.88 (dd, J=260.0, 15.0 Hz), 157.60, 156.85, 155.93 (d, J=14.0 Hz), 154.77 (dd, J=15.1, 7.7 Hz), 122.45 (d, J=10.1 Hz), 118.91, 114.13 (d, J=23.9 Hz), 105.39, 101.75 (dd, J=26.1, 4.5 Hz), 101.42 (dd, J=26.9, 23.0 Hz), 100.56 (d, J=21.3 Hz) 100.42 (d, J=27.6 Hz). ¹⁹F NMR (376 MHz, Chloroform-d) 6-104.46 (d, J=8.5 Hz), −112.27 (d, J=8.4 Hz), −114.33. HRMS (ESI-TOF) Calcd for C₁₅H₆F₃O₃[M+H]⁺: 291.0269; found: 291.0268.

### Synthesis of Compound 8

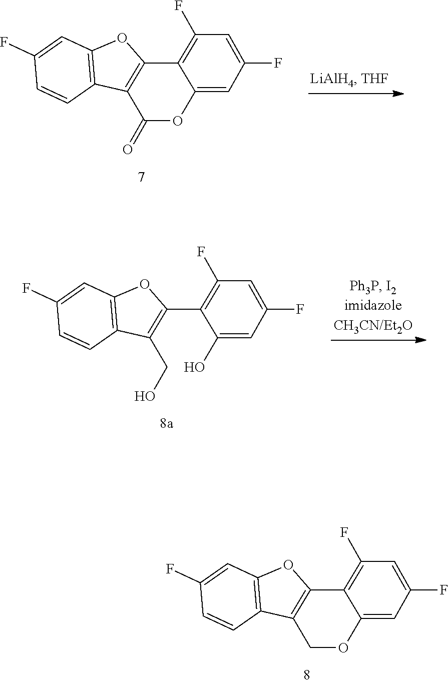 [SMILES: O=c1oc2cc(F)cc(F)c2c2oc3cc(F)ccc3c12 ; OCc1c(-c2c(O)cc(F)cc2F)oc2cc(F)ccc12 ; Fc1cc(F)c2c(c1)OCc1c-2oc2cc(F)ccc12]

To a solution of 7 (0.2 mmol) in THE (1.0 mL) was added lithium aluminum hydride solution (2.0 M in THF, 0.2 mL, 0.4 mmol) at 0° C. The reaction mixture was heated to 50° C. and stirred for 6 h. After cooling down to room temperature, the mixture was diluted with 10 mL THE and carefully quenched with 1 M aqueous HCl at 0° C. until the solution becomes clear. The aqueous layer was extracted with EtOAc. The combined organic layers were washed with brine then dried and evaporated under reduced pressure. The crude product was used in the next step without further purification.

To a solution of 8a, imidazole (40.8 mg, 0.6 mmol) and PPh₃ (104.8 mg, 0.4 mmol) in CH₃CN/Et₂O (1.0/1.0 mL) was added 12 (101.6 mg, 0.4 mmol) at 0° C. The mixture was stirred at ambient temperature for 12 h. Upon completion, the reaction mixture was diluted with saturated aqueous NH₄Cl solution and extracted with EtOAc three times. The combined organic layers were washed with brine then dried and evaporated under reduced pressure. The crude product was purified by flash chromatography (Hexane/EtOAc=20:1, v/v) to give product 8 as a white solid (30.0 mg, 54% yield in two steps).

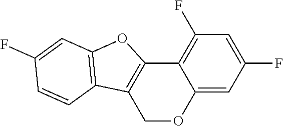 [SMILES: Fc1cc(F)c2c(c1)OCc1c-2oc2cc(F)ccc12]

### 1,3,9-trifluoro-6H-benzofuro[3,2-c]chromene (8)

¹H NMR (600 MHz, Chloroform-d) δ 7.33-7.27 (m, 2H), 7.04 (ddd, J=9.4, 8.6, 2.3 Hz, 1H), 6.54-6.46 (m, 2H), 5.58 (s, 2H). ¹³C NMR (151 MHz, Chloroform-d) δ 163.14 (dd, J=249.3, 14.8 Hz), 161.10 (d, J=243.9 Hz), 157.28 (dd, J=253.2, 15.1 Hz), 155.86 (dd, J=14.8, 6.6 Hz), 155.81 (dd, J=15.4 Hz), 145.13, 121.12, 119.03 (d, J=9.8 Hz), 111.98 (d, J=24.2 Hz), 107.74, 102.44 (dd, J=17.7, 3.9 Hz), 100.96 (dd, J=25.8, 3.8 Hz), 100.08 (d, J=27.0 Hz), 98.06 (dd, J=26.4, 24.8 Hz), 65.70. ¹⁹F NMR (376 MHz, Chloroform-d) 6-107.59 (d, J=6.5 Hz), −114.64 (d, J=6.4 Hz), −115.60. HRMS (ESI-TOF) Calcd for C₁₅H₈F₃O₂[M+H]⁺: 277.0476; found: 277.0472.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A method of C—H hydroxylation of benzoic and phenylacetic acids, comprising treating a benzoic or phenylacetic acid with a bidentate pyridone-carboxylic acid ligand and aqueous hydrogen peroxide in the presence of Pd(OAc)₂ and a base.

2. The method of claim 1, wherein the method of C—H hydroxylation of phenylacetic acids of Formula (1) occurs according to the following reaction scheme: 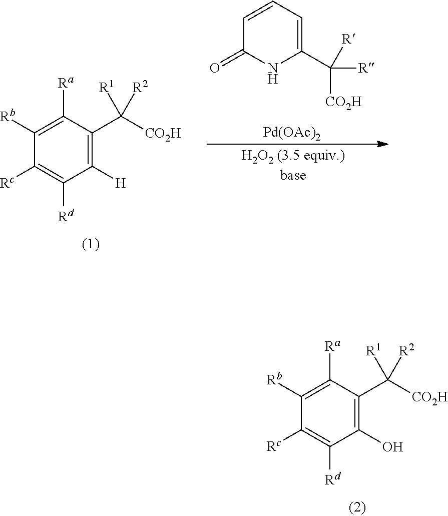
wherein:
R′ and R″ are independently (C₁-C₆)alkyl;
R¹ and R² are independently H, (C₁-C₆)alkyl, cycloalkyl, Ph, benzyl, OH, or —NC(═O)O(C₁-C₆)alkyl;
or R¹ and R² together form cyclopropyl;
or R¹ and R^{a} together form a 5- or 6-membered ring saturated or partially unsaturated carbocyclic or heterocyclic ring;
R^{a}, R^{b}, R^{c}, and R^{d} are independently H, halo, —(C₁-C₆)alkyl, halo (C₁-C₆)alkyl, hydroxy (C₁-C₆)alkyl, —O(C₁-C₆)alkyl, —(C₁-C₆)alkyl-O—(C₁-C₆)alkyl, —(C₁-C₆)alkyl-OC(═O)—(C₁-C₆)alkyl, —(C₁-C₆)alkyl-C(═O)O—(C₁-C₆)alkyl, —NH(C₁-C₆)alkyl, optionally substituted Ph, —C(═O)Ph, —C(═O)(C₁-C₆)alkyl, —NC(═O)(C₁-C₆)alkyl, —NC(═O)O(C₁-C₆)alkyl, —CF₃, —CN, —NO₂, cycloalkyl, —(C₁-C₆)alkylcyclocalkyl, or —(C₁-C₆)alkylcyclocalkanone;
or two adjacent members of R^{a}, R^{b}, R^{c}, and R^{d} together form a 5- or 6-membered aryl or partially unsaturated carbocyclic or heterocyclic ring, optionally substituted with one or more R^{x}; and
each R^{x} is independently halo, OH, —(C₁-C₆)alkyl, —O(C₁-C₆)alkyl, hydroxy (C₁-C₆)alkyl, —CF₃, —CHF₂, —CH₂F, —CN, —NO₂, cycloalkyl, or —(C₁-C₆)alkylcyclocalkyl.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. The method of claim 1, wherein the method of C—H hydroxylation of benzoic acids of Formula (3) is conducted according to the following reaction scheme: 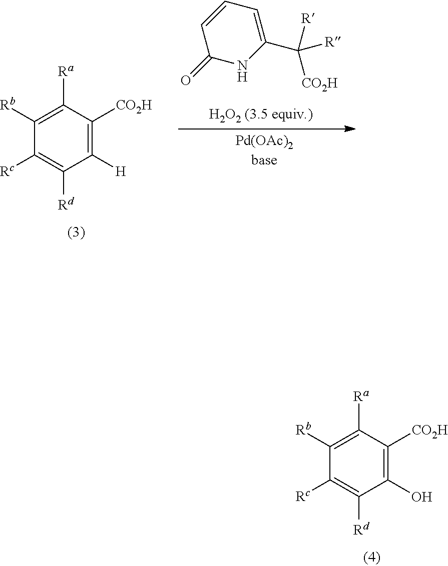
wherein:
R′ and R″ are independently (C₁-C₆)alkyl;
R^{a}, R^{b}, R^{c}, and R^{d} are independently H, halo, —(C₁-C₆)alkyl, halo (C₁-C₆)alkyl, hydroxy (C₁-C₆)alkyl, —O(C₁-C₆)alkyl, —(C₁-C₆)alkyl-O—(C₁-C₆)alkyl, —(C₁-C₆)alkyl-OC(═O)—(C₁-C₆)alkyl, —(C₁-C₆)alkyl-C(═O)O—(C₁-C₆)alkyl, —NH(C₁-C₆)alkyl, optionally substituted Ph, —C(═O)Ph, —C(═O)(C₁-C₆)alkyl, —NC(═O)(C₁-C₆)alkyl, —NC(═O)O(C₁-C₆)alkyl, —CF₃, —CN, —NO₂, cycloalkyl, —(C₁-C₆)alkylcyclocalkyl, or —(C₁-C₆)alkylcyclocalkanone;
or two adjacent members of R^{a}, R^{b}, R^{c}, and R^{d} together form a 5- or 6-membered aryl or partially unsaturated carbocyclic or heterocyclic ring, optionally substituted with one or more R^{x}; and
each R^{x} is independently halo, OH, —(C₁-C₆)alkyl, —O(C₁-C₆)alkyl, hydroxy (C₁-C₆)alkyl, —CF₃, —CHF₂, —CH₂F, —CN, —NO₂, cycloalkyl, or —(C₁-C₆)alkylcyclocalkyl.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The method of claim 1, wherein the base is K₂HPO₄, CsOAc, KOAc, KHCO₃, or NaOAc.

56. The method of claim 1, wherein the solvent is CH₃CN, DMA DMF, NMP, t-Amyl-OH, or DCE.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. The method of claim 2, wherein phenylacetic acid of Formula (1) is treated with 0.04 equivalents 2-methyl-2-(6-oxo-1,6-dihydropyridin-2-yl)propanoic acid, 0.02 equivalents Pd(OAc)₂, 3.5 equivalents H₂O₂, and 1.5 equivalents K₂HPO₄ in CH₃CN at room temperature for approximately 24 hours to form the hydroxylated phenyl acetic acid product of Formula (2).

69. The method of claim 6, wherein the hydroxylated phenyl acetic acid product of Formula (2) is selected from the group consisting of:
2-(2-hydroxy-4-(trifluoromethyl)phenyl)acetic acid (2a);
2-(2-hydroxy-4-methylphenyl)acetic acid (2b);
2-(2-hydroxyphenyl)acetic acid (2d);
2-(4-bromo-2-hydroxyphenyl)acetic acid (2f);
2-(4-chloro-2-hydroxyphenyl)acetic acid (2h);
2-(2-hydroxy-6-methylphenyl)acetic acid (2i);
2-(2-hydroxy-5-methylphenyl)acetic acid (2m);
2-(6-hydroxy-2,3-dimethylphenyl)acetic acid (2n);
2-(5-bromo-2-hydroxyphenyl)acetic acid (2o);
2-(5-chloro-2-hydroxyphenyl)acetic acid (2p);
2-(2-hydroxyphenyl)propanoic acid (2r);
2-cyclohexyl-2-(2-hydroxyphenyl)acetic acid (2s);
2-(2-hydroxyphenyl)-3-phenylpropanoic acid (2t);
2-hydroxy-2-(2-hydroxyphenyl)acetic acid (2v);
(S)-2-((tert-butoxycarbonyl)amino)-2-(2-hydroxyphenyl)acetic (2x);
4-hydroxy-2,3-dihydrobenzofuran-3-carboxylic acid (2y);
8-hydroxy-1,2,3,4-tetrahydronaphthalene-1-carboxylic acid (2z);
3,3-dimethylbenzofuran-2(3H)-one (2ab′);
1-(2-hydroxyphenyl)cyclopropane-1-carboxylic acid (2ac);
1-(4-chloro-2-hydroxyphenyl)cyclopropane-1-carboxylic acid (2ad);
2-(2-hydroxy-4-isobutylphenyl)propanoic acid (2ae);
2-(5-benzoyl-2-hydroxyphenyl)propanoic acid (2af);
2-(2-fluoro-5-hydroxy-[1,1′-biphenyl]-4-yl)propanoic acid (2ag);
2-(2-hydroxy-4-((2-oxocyclopentyl)methyl)phenyl)propanoic acid (2ai); and
(S)-2-(3-hydroxy-6-methoxynaphthalen-2-yl)propanoic acid (2aj).

70. The method of claim 2, wherein 0.5 mmol phenylacetic acid of Formula (1) is treated with 0.04 equivalents 2-methyl-2-(6-oxo-1,6-dihydropyridin-2-yl)propanoic acid, 0.02 equivalents Pd(OAc)₂, 3.5 equivalents H₂O₂, and 1.5 equivalents K₂HPO₄ in CH₃CN at room temperature for approximately 24 hours to form the hydroxylated phenyl acetic acid product of Formula (2).

71. The method of claim 2, wherein the hydroxylated phenyl acetic acid product of Formula (2) is selected from the group consisting of:
1-(3′,4′-dichloro-2-fluoro-5-hydroxy-[1,1′-biphenyl]-4-yl)cyclopropane-1-carboxylic acid (2ak); and
2-((8R,9S,13S,14S)-2-hydroxy-13-methyl-17-oxo-7,8,9,11,12,13,14,15,16,17-decahydro-6H-cyclopenta[a]phenanthren-3-yl)acetic acid (2al).

72. The method of claim 2, wherein 1 mmol phenylacetic acid of Formula (1) is treated with 0.04 equivalents 2-methyl-2-(6-oxo-1,6-dihydropyridin-2-yl)propanoic acid, 0.02 equivalents Pd(OAc)₂, 3.5 equivalents H₂O₂, and 1.5 equivalents K₂HPO₄ in CH₃CN at room temperature for approximately 24 hours to form the hydroxylated phenyl acetic acid product of Formula (2).

73. The method of claim 2, wherein the hydroxylated phenyl acetic acid product of Formula (2) is selected from the group consisting of:
2-(4-cyano-2-hydroxyphenyl)acetic acid (2j);
2-(2-hydroxy-4-nitrophenyl)acetic acid (2k); and
2-(4-bromo-2-hydroxyphenyl)-2-hydroxyacetic (2w).

74. The method of claim 2, wherein 1 mmol phenylacetic acid of Formula (1) is treated with 0.04 equivalents 2-methyl-2-(6-oxo-1,6-dihydropyridin-2-yl)propanoic acid, 0.02 equivalents Pd(OAc)₂, 3.5 equivalents H₂O₂, and 2.0 equivalents KHCO₃ in DMA at room temperature for approximately 24 hours to form the hydroxylated phenyl acetic acid product of Formula (2).

75. The method of claim 2, wherein the hydroxylated phenyl acetic acid product of Formula (2) is selected from the group consisting of:
2-(2-hydroxy-4-methoxyphenyl)acetic acid (2c);
2-(3-hydroxy-[1,1′-biphenyl]-4-yl)acetic acid (2e);
2-(2-hydroxy-4-iodophenyl)acetic acid (2g);
2-(4-fluoro-2-hydroxyphenyl)acetic acid (2i);
2-(3-hydroxynaphthalen-2-yl)acetic acid (2q);
2-(2-hydroxyphenyl)-2-phenylacetic acid (2u);
2-(3-hydroxydibenzo[b,d]furan-2-yl)acetic acid (2aa);
2-(4-acetamido-2-hydroxyphenyl)acetic acid (2ah); and
(S)-2-(3-hydroxy-6-methoxynaphthalen-2-yl)propanoic acid (2aj).

76. The method of claim 3, wherein benzoic acid of Formula (3) is treated with 0.04 equivalents 2-methyl-2-(6-oxo-1,6-dihydropyridin-2-yl)propanoic acid, 0.02 equivalents Pd(OAc)₂, 3.5 equivalents H₂O₂, and 1.5 equivalents K₂HPO₄·3H₂O in CH₃CN at room temperature for approximately 24 hours to form the hydroxylated benzoic acid product of Formula (4).

77. The method of claim 14, wherein the hydroxylated benzoic acetic acid product of Formula (4) is selected from the group consisting of:
2-hydroxybenzoic acid (4a);
2-hydroxy-4-methylbenzoic acid (4b);
2-hydroxy-5-methylbenzoic acid (4c);
2-hydroxy-6-methylbenzoic acid (4d);
4-hydroxy-[1,1′-biphenyl]-3-carboxylic acid (4e);
3-hydroxy-[1,1′-biphenyl]-2-carboxylic acid (4f);
2-hydroxy-1-naphthoic acid (4g);
2-hydroxy-4-methoxy-6-methylbenzoic acid (4i);
4-hydroxy-2,3-dihydrobenzofuran-5-carboxylic (4j);
6-hydroxy-2,3-dihydrobenzofuran-5-carboxylic acid (4j′); and
7-hydroxychromane-6-carboxylic acid (4k).

78. The method of claim 3, wherein the benzoic acid of Formula (3) is treated with 0.04 equivalents 2-methyl-2-(6-oxo-1,6-dihydropyridin-2-yl)propanoic acid, 0.02 equivalents Pd(OAc)₂, 3.5 equivalents H₂O₂, and 1.5 equivalents K₂HPO₄·3H₂O in CH₃CN at approximately 60° C. for approximately 24 hours to form the hydroxylated benzoic acid product of Formula (4).

79. The method of claim 16, wherein the hydroxylated benzoic acetic acid product of Formula (4) is selected from the group consisting of:
2-hydroxy-5-(trifluoromethyl)benzoic acid (4h); and
2′,4′-difluoro-4-hydroxy-[1,1′-biphenyl]-3-carboxylic acid (41).

80. The method of claim 3, wherein benzoic acid of Formula (3) is treated with 0.1 equivalents 2-methyl-2-(6-oxo-1,6-dihydropyridin-2-yl)propanoic acid, 0.05 equivalents Pd(OAc)₂, 3.5 equivalents H₂O₂, and 1.5 equivalents CsOAc in DMA at 60° C. for approximately 24 hours to form the hydroxylated benzoic acid product of Formula (4).

81. The method of claim 18, wherein the hydroxylated benzoic acetic acid product of Formula (4) is selected from the group consisting of:
2,4-difluoro-6-hydroxybenzoic acid (4m);
4,5-difluoro-2-hydroxybenzoic acid (4n);
3,5-difluoro-2-hydroxybenzoic acid (4o);
2-fluoro-6-hydroxybenzoic acid (4p);
2-hydroxy-4-nitrobenzoic acid (4q);
4-acetyl-2-hydroxybenzoic acid (4r);
2-hydroxy-4-methoxybenzoic acid (4s);
2-hydroxy-6-(4-methylbenzoyl)benzoic acid (4u); and
2-((3-chloro-2-methylphenyl)amino)-6-hydroxybenzoic acid (4v).

82. The method of claim 3, wherein benzoic acid of Formula (3) is treated with 0.1 equivalents 2-methyl-2-(6-oxo-1,6-dihydropyridin-2-yl)propanoic acid, 0.05 equivalents Pd(OAc)₂, 3.5 equivalents H₂O₂, and 1.5 equivalents K₂HPO₄·3H₂O in DMA at 60° C. for approximately 48 hours to form the hydroxylated benzoic acid product of Formula (4).

83. The method of claim 20, wherein the hydroxylated benzoic acetic acid product of Formula (4) is 2-bromo-4-fluoro-6-hydroxybenzoic acid (4t).

84. The method of claim 3, wherein 0.5 mmol benzoic acid of Formula (3) is treated with 0.1 equivalents 2-methyl-2-(6-oxo-1,6-dihydropyridin-2-yl)propanoic acid, 0.05 equivalents Pd(OAc)₂, 3.5 equivalents H₂O₂, and 1.5 equivalents K₂HPO₄·3H₂O in DMA at room temperature for approximately 24 hours to form the hydroxylated benzoic acid product of Formula (4).

85. The method of claim 22, wherein the hydroxylated benzoic acetic acid product of Formula (4) is (8R,9S,13S,14S)-2-hydroxy-13-methyl-17-oxo-7,8,9,11,12,13,14,15,16,17-decahydro-6H-cyclopenta[a]phenanthrene-3-carboxylic acid (4w).

86. A method of preparing trifluorinated coumestan and pterocarpene according to the following reaction scheme:

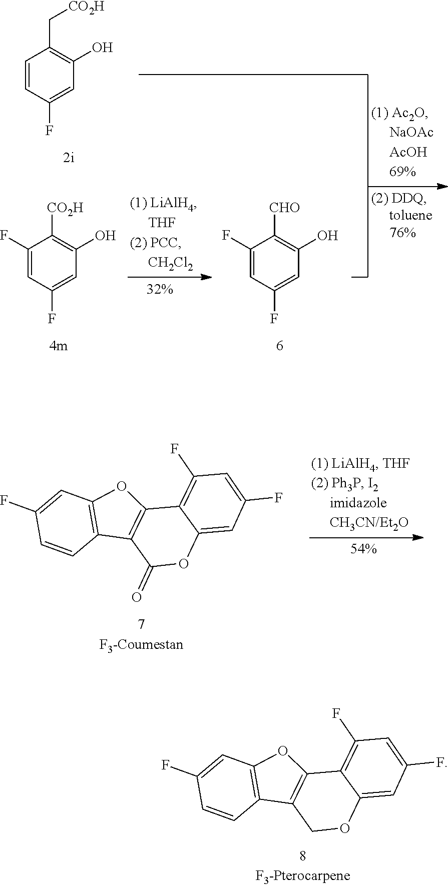 [SMILES: O=C(O)Cc1ccc(F)cc1O ; O=C(O)c1c(O)cc(F)cc1F ; O=Cc1c(O)cc(F)cc1F ; O=c1oc2cc(F)cc(F)c2c2oc3cc(F)ccc3c12 ; Fc1cc(F)c2c(c1)OCc1c-2oc2cc(F)ccc12]

87. A method of hydroxylating ibuprofen according to the following reaction scheme:

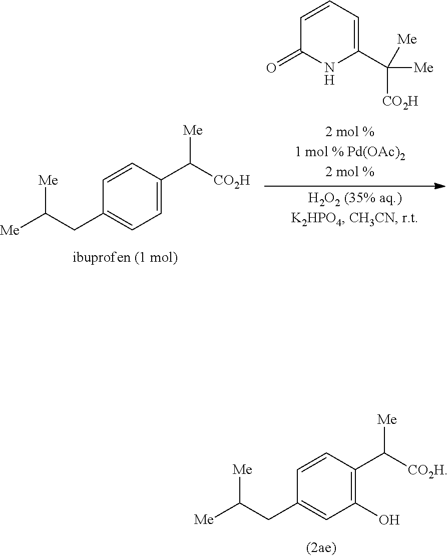 [SMILES: CC(C)(C(=O)O)c1cccc(=O)[nH]1 ; CC(C)Cc1ccc(C(C)C(=O)O)cc1 ; CC(C)Cc1ccc(C(C)C(=O)O)c(O)c1]

88. (canceled)

89. A method of lignin depolymerization, comprising treating lignin with a bidentate pyridone-carboxylic acid ligand and aqueous hydrogen peroxide in the presence of Pd(OAc)₂ and a base.

90. A method of lignin depolymerization, comprising treating lignin with 2-methyl-2-(6-oxo-1,6-dihydropyridin-2-yl)propanoic acid and aqueous hydrogen peroxide in the presence of Pd(OAc)₂ and a base.
